SEQUENCE LISTING (36 sequences)

SEQ ID NO: 1 - RNA, 21 nt, Homo sapiens
cugugcgugugacagcggcug

SEQ ID NO: 2 - DNA, 1965 bp, Homo sapiens
gagagggtgccagcggccgcagctgaagttgggccgagagccggcgacggccccgcgccggggtcgcaggcctgcaggagttgagggctgcacctgctcgctggagagggagaggcagatttagtggacgcctggcatggactcggactggcctttggaagctccctgccctgacggggttgcctgtcaccactgcgaagtgaggcttggcaggacctgcacctgagaaaggccgtgtgtggtcttggggtccacacctgcagagctaacttactgccagacggcgacttactgtgggccaccctcagtgaaccggggtgtcctcagctggccctacagagcacttctgtgctggggatgagtaggaattctgggcgaggagggtcccagcgccgcccctcgatacagcctggctctgccctctgcccgtacttataccaggtgggatccctgccctgcattgcctggggattggctgggcttgggcccgccctgctgtggaactggatgttttcagggagcccagcctttcctcatgtcaacacagttcacaatatagttttcaaagtacagtttaaaactcaaaagtaaacttttcagcaactcaaaggtttgctgagtgatctgaagcactctggccactttttggggccatgggatttggttcacctgaaacagccagtgagaggccgggtgtggtggctcacacccgtaatcccaacacttcaggaggcagacgcgggtgatcgctcacttgagatcaggagttcaagaccagcctgggcaacatggtgaaacctcgtctctactaaaaatacaaaaattagctaggcatggtggtgggcacctgtaatcccagctacttggaaggctgaggcaagagaatcgcttgaacctgggaggtggaggttgcagcgagacgagattacgccgctgcactccagcctgggtgacgagagactctgcctcaaaaaaataaaaaaatgaaacagccagtgaggaggaaggctccccgccttccccccgccggaacatagccatagctgctgctgggacaccctcttggtggggaagaagactggttagcttcatcagagccagcagcagcagaccagggacgggcacctaggcagtggcctcagagtgaacaggagttcctcagaaacacacacagggacggcgtggcgcatgctctgccagctccatgcctccttcccattgtggggctgggctacgtagggcagagctcatgacctccgggaggacatgggggtgggctctggatggcacctggcattgccccctgctggcctatgtgacggtgtggagggctggtcacagaggtacgaccatcccttcagaatgtgggtcggggctgtggatggaggagtaggcccctcatatcccaggcctgctgcccaggcacaacccacttggcctatgcattccaggctccatcccatgtgactctgggcttagccccttctggggccacaggtcaggcaggtccaggccccaaggacctcccagtgacaggcgactgtgagctgggcagacaggagtgaagtcaggtgggggttctggcttgctgacaccagcgtttggagcctcctgctgctgcctggcttccctgcattccctgttccctgcctcaggcaagaaataaccaagccgagttgcctctgcacagcagtgagctcctggtggccctggcttctggggagccctgtggatggcttccttgcccaagtcaaggccttcttgttccctttgtgtgctccagagaaagggggcagcaccagatccagatccagggccaaccaacagaaagctgagtccatcccaaactcgcccattctcagagcacaaagaccccatgatctagggcaaacttgtccaactgttggcccatggaacagctttgaatgcagcccaacacaaatctataaattttcttaaacattg

SEQ ID NO: 3 - RNA, 110 nt, Homo sapiens
acccggcagugccuccaggcgcagggcagccccugcccaccgcacacugcgcugccccagacccacugugcgugugacagcggcugaucugugccugggcagcgcgaccc

SEQ ID NO: 4 - RNA, 12 nt, Homo sapiens
cugugcguguga

SEQ ID NO: 5 - RNA, 21 nt, Artificial Sequence
  Synthetic
cugucggugugacagcggcug

SEQ ID NO: 6 - RNA, 21 nt, Artificial Sequence
  Synthetic
guccuuaaaccgguugaaunn

SEQ ID NO: 7 - RNA, 21 nt, Artificial Sequence
  Synthetic
gcaacuugaggaaguaccann

SEQ ID NO: 8 - RNA, 21 nt, Artificial Sequence
  Synthetic
ccuaauagucccagugaaunn

SEQ ID NO: 9 - RNA, 21 nt, Artificial Sequence
  Synthetic
ggcucaaggagaucguuuann

SEQ ID NO: 10 - RNA, 21 nt, Artificial Sequence
  Synthetic
gaauggacuuggcucuguann

SEQ ID NO: 11 - RNA, 21 nt, Artificial Sequence
  Synthetic
gccacagucugaaugguuunn

SEQ ID NO: 12 - RNA, 21 nt, Artificial Sequence
  Synthetic
cgggccaggugaaagucuann

SEQ ID NO: 13 - RNA, 21 nt, Artificial Sequence
  Synthetic
gcgacagcuggaguaugaann

SEQ ID NO: 14 - RNA, 21 nt, Artificial Sequence
  Synthetic
gcuucagugccaugacaaann

SEQ ID NO: 15 - DNA, 21 bp, Artificial Sequence
  Synthetic
cguccaaucugagcgugcutt

SEQ ID NO: 16 - DNA, 21 bp, Artificial Sequence
  Synthetic
gcuggcacgugagaagauutt

SEQ ID NO: 17 - DNA, 21 bp, Artificial Sequence
  Synthetic
gguacauccagucccugaatt

SEQ ID NO: 18 - DNA, 21 bp, Artificial Sequence
  Synthetic
gcuuguaccugcaggaucutt

SEQ ID NO: 19 - DNA, 21 bp, Artificial Sequence
  Synthetic
cgauguuguuucuguggaatt

SEQ ID NO: 20 - DNA, 21 bp, Artificial Sequence
  Synthetic
cgagaacaguugaaacacatt

SEQ ID NO: 21 - DNA, 20 bp, Artificial Sequence
  Synthetic
aggagccttgacggtttgac

SEQ ID NO: 22 - DNA, 19 bp, Artificial Sequence
  Synthetic
gaggaccagggtgacagtg

SEQ ID NO: 23 - DNA, 2377 bp, Homo sapiens
  CDS (525)..(1889)
acccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaaaaaaaaaaaaaaaaaaaa

SEQ ID NO: 24 - protein, 454 aa, Homo sapiens
LDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 25 - DNA, 2613 bp, Homo sapiens
  CDS (301)..(1695)
gtcatctgtctggacgcgctgggtggatgcggggggctcctgggaactgtgttggagccgagcaagcgctagccaggcgcaagcgcgcacagactgtagccatccgaggacacccccgcccccccggcccacccggagacacccgcgcagaatcgcctccggatcccctgcagtcggcgggagtgttggaggtcggcgccggcccccgccttccgcgccccccacgggaaggaagcacccccggtattaaaacgaacggggcggaaagaagccctcagtcgccggccgggaggcgagccgatgccgagctgctccacgtccaccatgccgggcatgatctgcaagaacccagacctcgagtttgactcgctacagccctgcttctacccggacgaagatgacttctacttcggcggccccgactcgacccccccgggggaggacatctggaagaagtttgagctgctgcccacgcccccgctgtcgcccagccgtggcttcgcggagcacagctccgagcccccgagctgggtcacggagatgctgcttgagaacgagctgtggggcagcccggccgaggaggacgcgttcggcctggggggactgggtggcctcacccccaacccggtcatcctccaggactgcatgtggagcggcttctccgcccgcgagaagctggagcgcgccgtgagcgagaagctgcagcacggccgcgggccgccaaccgccggttccaccgcccagtccccgggagccggcgccgccagccctgcgggtcgcgggcacggcggggctgcgggagccggccgcgccggggccgccctgcccgccgagctcgcccacccggccgccgagtgcgtggatcccgccgtggtcttcccctttcccgtgaacaagcgcgagccagcgcccgtgcccgcagccccggccagtgccccggcggcgggccctgcggtcgcctcgggggcgggtattgccgccccagccggggccccgggggtcgcccctccgcgcccaggcggccgccagaccagcggcggcgaccacaaggccctcagtacctccggagaggacaccctgagcgattcagatgatgaagatgatgaagaggaagatgaagaggaagaaatcgacgtggtcactgtggagaagcggcgttcctcctccaacaccaaggctgtcaccacattcaccatcactgtgcgtcccaagaacgcagccctgggtcccgggagggctcagtccagcgagctgatcctcaaacgatgccttcccatccaccagcagcacaactatgccgccccctctccctacgtggagagtgaggatgcacccccacagaagaagataaagagcgaggcgtccccacgtccgctcaagagtgtcatccccccaaaggctaagagcttgagcccccgaaactctgactcggaggacagtgagcgtcgcagaaaccacaacatcctggagcgccagcgccgcaacgaccttcggtccagctttctcacgctcagggaccacgtgccggagttggtaaagaatgagaaggccgccaaggtggtcattttgaaaaaggccactgagtatgtccactccctccaggccgaggagcaccagcttttgctggaaaaggaaaaattgcaggcaagacagcagcagttgctaaagaaaattgaacacgctcggacttgctagacgcttctcaaaactggacagtcactgccactttgcacattttgattttttttttaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttctttttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggaagttcatacctaagtactgtaataatacctcaatgtttgaggagcatgttttgtatacaaatatattgttaatctctgttatgtactgtactaattcttacactgcctgtatactttagtatgacgctgatacataactaaatttgatacttatattttcgtatgaaaatgagttgtgaaagttttgagtagatattactttatcactttttgaactaagaaacttttgtaaagaaatttactatatatatatgcctttttcctagcctgtttcttcctgttaatgtatttgttcatgtttggtgcatagaactgggtaaatgcaaagttctgtgtttaatttcttcaaaatgtatatatttagtgctgcatcttatagcactttgaaatacctcatgtttatgaaaataaatagcttaaaattaaatgaaaaaaaaa

SEQ ID NO: 26 - protein, 464 aa, Homo sapiens
MPSCSTSTMPGMICKNPDLEFDSLQPCFYPDEDDFYFGGPDSTPPGEDIWKKFELLPTPPLSPSRGFAEHSSEPPSWVTEMLLENELWGSPAEEDAFGLGGLGGLTPNPVILQDCMWSGFSAREKLERAVSEKLQHGRGPPTAGSTAQSPGAGAASPAGRGHGGAAGAGRAGAALPAELAHPAAECVDPAVVFPFPVNKREPAPVPAAPASAPAAGPAVASGAGIAAPAGAPGVAPPRPGGRQTSGGDHKALSTSGEDTLSDSDDEDDEEEDEEEEIDVVTVEKRRSSSNTKAVTTFTITVRPKNAALGPGRAQSSELILKRCLPIHQQHNYAAPSPYVESEDAPPQKKIKSEASPRPLKSVIPPKAKSLSPRNSDSEDSERRRNHNILERQRRNDLRSSFLTLRDHVPELVKNEKAAKVVILKKATEYVHSLQAEEHQLLLEKEKLQARQQQLLKKIEHARTC

SEQ ID NO: 27 - DNA, 3622 bp, Homo sapiens
  CDS (582)..(1676)
aatgcgcctgcagctcgcgctcccgcgccgatcccgagagcgtccgggccgccgtgcgcgagcgagggagggcgcgcgcgcggggggggcgcgcttgtgagtgcgggccgcgctctcggcggcgcgcatgtgcgtgtgtgctggctgccgggctgccccgagccggcggggagccggtccgctccaggtggcgggcggctggagcgaggtgaggctgcgggtggccagggcacgggcgcgggtcccgcggtgcgggctggctgcaggctgccttctgggcacggcgcgcccccgcccggccccgccgggccctgggagctgcgctccgggcggcgctggcaaagtttgctttgaactcgctgcccacagtcgggtccgcgcgctgcgattggcttcccctaccactctgacccggggcccggcttcccgggacgcgaggactgggcgcaggctgcaagctggtggggttggggaggaacgagagcccggcagccgactgtgccgagggacccggggacacctccttcgcccggccggcacccggtcagcacgtccccccttccctcccgcagggagcggacatggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcggagaatgaagaaattgatgttgtgacagtagagaagaggcagtctctgggtattcggaagccggtcaccatcacggtgcgagcagaccccctggatccctgcatgaagcatttccacatctccatccatcagcaacagcacaactatgctgcccgttttcctccagaaagctgctcccaagaagaggcttcagagaggggtccccaagaagaggttctggagagagatgctgcaggggaaaaggaagatgaggaggatgaagagattgtgagtcccccacctgtagaaagtgaggctgcccagtcctgccaccccaaacctgtcagttctgatactgaggatgtgaccaagaggaagaatcacaacttcctggagcgcaagaggcggaatgacctgcgttcgcgattcttggcgctgagggaccaggtgcccaccctggccagctgctccaaggcccccaaagtagtgatcctaagcaaggccttggaatacttgcaagccctggtgggggctgagaagaggatggctacagagaaaagacagctccgatgccggcagcagcagttgcagaaaagaattgcatacctcactggctactaactgaccaaaaagcctgacagttctgtcttacgaagacacaagtttattttttaacctccctctcccctttagtaatttgcacattttggttatggtgggacagtctggacagtagatcccagaatgcattgcagccggtgcacacacaataaaggcttgcattcttggaaaccttgaaacccagctctccctcttccctgactcatgggagtgctgtatgttctctggcgcctttggcttcccagcaggcagctgactgaggagccttggggtctgcctagctcactagctctgaagaaaaggctgacagatgctatgcaacaggtggtggatgttgtcaggggctccagcctgcatgaaatctcacactctgcatgagctttaggctaggaaaggatgctcccaactggtgtctctggggtgatgcaaggacagctgggcctggatgctctccctgaggctcctttttccagaagacacacgagctgtcttgggtgaagacaagcttgcagacttgatcaacattgaccattacctcactgtcagacactttacagtagccaaggagttggaaacctttatatattatgatgttagctgacccccttcctcccactcccaatgctgcgaccctgggaacacttaaaaagcttggcctctagattctttgtctcagagccctctgggctctctcctctgagggagggacctttctttcctcacaagggacttttttgttccattatgccttgttatgcaatgggctctacagcaccctttcccacaggtcagaaatatttccccaagacacagggaaatcggtcctagcctggggcctggggatagcttggagtcctggcccatgaacttgatccctgcccaggtgttttccgaggggcacttgaggcccagtcttttctcaaggcaggtgtaagacacctcagagggagaactgtactgctgcctctttcccacctgcctcatctcaatccttgagcggcaagtttgaagttcttctggaaccatgcaaatctgtcctcctcatgcaattccaaggagcttgctggctctgcagccacccttgggccccttccagcctgccatgaatcagatatctttcccagaatctgggcgtttctgaagttttggggagagctgttgggactcatccagtgctccagaaggtggacttgcttctggtgggttttaaaggagcctccaggagatatgcttagccaaccatgatggattttaccccagctggactcggcagctccaagtggaatccacgtgcagcttctagtctgggaaagtcacccaacctagcagttgtcatgtgggtaacctcaggcacctctaagcctgtcctggaagaaggaccagcagcccctccagaactctgcccaggacagcaggtgcctgctggctctgggtttggaagttggggtgggtagggggtggtaagtactatatatggctctggaaaaccagctgctacttccaaatctattgtccataatggtttctttctgaggttgcttcttggcctcagaggaccccaggggatgtttggaaatagcctctctacccttctggagcatggtttacaaaagccagctgacttctggaattgtctatggaggacagtttgggtgtaggttactgatgtctcaactgaatagcttgtgttttataagctgctgttggctattatgctgggggagtcttttttttttatattgtatttttgtatgccttttgcaaagtggtgttaactgtttttgtacaaggaaaaaaactcttggggcaatttcctgttgcaagggtctgatttattttgaaaggcaagttcacctgaaattttgtatttagttgtgattactgattgcctgattttaaaatgttgccttctgggacatcttctaataaaagatttctcaaacatg

SEQ ID NO: 28 - protein, 364 aa, Homo sapiens
MDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEEVLERDAAGEKEDEEDEEIVSPPPVESEAAQSCHPKPVSSDTEDVTKRKNHNFLERKRRNDLRSRFLALRDQVPTLASCSKAPKVVILSKALEYLQALVGAEKRMATEKRQLRCRQQQLQKRIAYLTGY

SEQ ID NO: 29 - DNA, 4865 bp, Homo sapiens
  CDS (253)..(2001)
gcagtgccgccttttttttttttttgcatcccatttttttaaatttgcaattttatattttgcaaatattttgagagacattgatttttctccccgtgctcccccgttcttccctgcggagtgcgctgcgccgcccagccctgtcgccccccggaggtgatccctccctcctgcctgcccgccagcctgacctgtgcccggctcgcgggccgcagcctcggccccggcgcgcccccggcagctctcggcgcgatgagcatagagacgctactggaggcggcccgcttcctggaatggcaagcgcagcaacaacagagagcacgtgaggagcaggagcggcttcgcttggagcaggagcgagagcaggaacagaagaaggccaatagcctggccaggctggcacatacccttcctgtggaggaaccccgcatggaggcgccacccctgcctctgtctccaccggctcccccgccggcacccccaccaccacttgccacccctgccccactgactgtcatccctatccctgtggtgaccaactcccctcagcctctacccccacccccacccttgcccgcggcagcccagcctctgcccctggcgcctcgtcagccggccctggttggcgcccccggactcagcattaaggagcctgcccccctgcccagcaggccgcaggtgcccacccctgctcccctactgccggactcgaaggccaccattccacccaatggcagccccaagcctttgcagcccctccccacgcctgtcctgaccatagcgccacaccctggagtccagcctcagctggccccccagcagccgcccccacccacgctggggaccctgaagttggcaccagctgaagaagtcaaatccagtgaacagaagaagaggcccggggggatcggaaccagagaagtccacaacaaattggagaagaacaggagggcccatctgaaagagtgctttgagaccctgaagcggaacatccccaacgtggatgacaagaagacgtccaatctgagcgtgctgcggacggcgctgcggtacatccagtccctgaagaggaaggagaaggaatatgagcatgaaatggagcggctggcacgtgagaagattgccacgcagcagcggctggcagagctcaagcacgagctgagccagtggatggacgtactggagattgaccgcgtgctgcggcagacgggccagcccgaggatgaccaggcctccacctccaccgcctctgagggtgaggacaacatagacgaggatatggaggaggaccgggcgggcctgggcccacctaagctgagccatcgtccccagccggagctgctgaagtccaccctgccaccccccagcaccacccctgcgcctctgcctccacacccacaccctcacccccactccgtggccctacctcctgcccacctccccgtgcagcagcagcagccacagcagaagacccctctgccagcccctcctcccccaccggctgcccctgcccagacactggtgccagctccagcccatctggtggcgacggctgggggtggctccacggtcatcgcccacacagccaccactcacgcttcagtcatccagactgtgaaccacgttctgcaggggccaggcggcaagcacatcgcccacatcgccccctcggcccccagccctgcggtgcaactggcgcctgccacaccccccattgggcacatcactgtgcaccctgccaccctcaaccatgtggcccacctgggctcccagctgcccttgtacccgcagcccgtggcagtgagccacatcgcccacaccctctcgcaccagcaagtcaacggcacggccggcctggggcccccggctactgtcatggcaaagccggccgtgggggctcaggtggtgcaccacccccagctggtgggccagaccgtgctcaaccctgtgaccatggtcaccatgccctccttcccagtcagcacactcaagctggcttgaggacgaggccactcagaggcccccagtggggacagggagggggacctgtcccccactctctcacccaccagctccacacattccagccaggcccaggccagcccccccacccacccccaggcctcctaggggaagggggtgcaaagactctgagccaagggagggaagggccaccctgctgcactaggacttggtaagatgactctgagaaaatgcgagactctgatggaatgtgccacctgtccggcccagtgccagctccagtgccgctcctgcttcccctccctaccctcggaaatcagtgcgatgtggacgtcacgctccctgacttctcccccgccctgccccgccttcgtgtgctgctgctgctattgctgtctggtgaggtggcccaggcccccggctttcctccggagcctcatgttctcttcccaggcctttggaggggaaatggggaaagcagaactgaagccacttggcccagaaagctgcggattggggtgataaggggccttgctctgagcacaggtgacagatcataggaagtggctggtctggagtcccacccggacaggtggggcctcagcctggggctctctgacccggttgcagtcactgtgattcgttaccgtagatactacttaaaatgattctctaccaacaataaaccaaacccagccactgccaagcttcctgtgccctcaccccaatccctgccactgggctctggacctcaggagggcaggctgaggtggggaaggagggacttggctgtccttccccttccccgtcccctgcagcctgggtctggatgggaggagagccactgggcccctgtccccagtccccagtccccagccctgggcttggctcttggcttccagaaggcagcaaagaggggcgctgtcctgcgttcagcccctgatctctgacctctgctgagtgctgggcactgctccaagggacaggtgggcctggcggcctgtgggtttgggtgcctcctgcagtttgggagacatggaccagcatctggtcttgtttccaggagcatagaagccacatcgttgagacatcaggaaggtaaaaacccagcggcttagccaagccctaagcctgtccccagaccaaccctgggacctatacagaacagagggccagagctagggctgctgcttctgctccagcccctttgcctctgtcctcccatcccctcaacaccctgcttctcccggggacgcttttgagtgggccctgcccggggagctgcagagcagcagcacctttctctgagaagaggtccttggttgggtcaaggacagggctgagcgtggaagggggaggagtcaggggctctgtgttaggatgcggctttctctgcctctgggcagcctgctttggcctttccttgtatgtgggtgtttattacaagtggctttgtgtcagacacgctcggctccccacctggacacacactcaccagtggcctttcagtgtagcgggaggaagccggtgtccctggatgtgaagctcacactgatgggctggggcaggggcctgggccggcgagggggccggggggaggggacagagctgaggatttcctggagtgccctgcaggcacagaggaggtcagcaaaggtcttgaatagattttctctggaaataaagaatccttagatgcctaaaaattcccttcctgttccctcctggtcctggacacctcccaggggactgttccttatttctctctcctggtgtgggtaaagggacagttacaaaccaggtcaccatcctcagaggctgagccctgtacccaccccagcacagccacctccgccagaccccgtggctctgggagagccagctttgctattcccattgtgacagcgatggcaggcctgacccacggggcagttagagcagcctcgtggagctcgcccctcccctcagcctgcccacctctccctgcatcctgaggccagtggcccccgggcctgcacagatacctcatcatccacccgctgcccctccccgtccctgtcccccagcatcgggggcctgcaaatctagtgccgaatgactatgtccagattggtgacgatgggtgttgctgtttttcttgtcgtttgtgaacgctgtgatgctgtgtgcccaagtgggcagtcaccacccagcccttccttgggcgtctcccccagagtgctgtcgggaccacatctgtcctcaccctgtgggaccgcctggccctccctccctagcccttccagcctgggacacacacacacacacacacacacacacacgcacgcacacgcacacatcttacctctcatgcgtgttttaccttttgatgttcagagtggctcactggctgggagtccttacctcggggagagggggaggttggttccttggggggccaaagaaggcagggaatgcctggagggtaactggggggccaccatgaccccttctctctccagaaacagctgcttctccccccatcccagggtcccacccccaacccccagaggtggcccttgtttacagtgaggactcggccactgtgtctctgtttcctgaaatataaactgtagcgaccccagactgtagagatttttatgtgtttggatacatctgctgtgtggaaaaaaaaaaaaactacaaaaaccctaattttgtacatactgtatttttactattgaactgtattctagtggctgttcatgctccaagactttagttaccgagacatgaatactatccatgtaataagcacttgcctggaataaaatataaaactgaaataaacctgcactgaaacctgagatggagctgccaaaaaaaaaaaaaaaaaaaaaaaa

SEQ ID NO: 30 - protein, 582 aa, Homo sapiens
MSIETLLEAARFLEWQAQQQQRAREEQERLRLEQEREQEQKKANSLARLAHTLPVEEPRMEAPPLPLSPPAPPPAPPPPLATPAPLTVIPIPVVTNSPQPLPPPPPLPAAAQPLPLAPRQPALVGAPGLSIKEPAPLPSRPQVPTPAPLLPDSKATIPPNGSPKPLQPLPTPVLTIAPHPGVQPQLAPQQPPPPTLGTLKLAPAEEVKSSEQKKRPGGIGTREVHNKLEKNRRAHLKECFETLKRNIPNVDDKKTSNLSVLRTALRYIQSLKRKEKEYEHEMERLAREKIATQQRLAELKHELSQWMDVLEIDRVLRQTGQPEDDQASTSTASEGEDNIDEDMEEDRAGLGPPKLSHRPQPELLKSTLPPPSTTPAPLPPHPHPHPHSVALPPAHLPVQQQQPQQKTPLPAPPPPPAAPAQTLVPAPAHLVATAGGGSTVIAHTATTHASVIQTVNHVLQGPGGKHIAHIAPSAPSPAVQLAPATPPIGHITVHPATLNHVAHLGSQLPLYPQPVAVSHIAHTLSHQQVNGTAGLGPPATVMAKPAVGAQVVHHPQLVGQTVLNPVTMVTMPSFPVSTLKLA

SEQ ID NO: 31 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 32 - protein, 735 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGII

SEQ ID NO: 33 - protein, 789 aa, Homo sapiens
MAATTANPEMTSDVPSLGPAIASGNSGPGIQGGGAIVQRAIKRRPGLDFDDDGEGNSKFLRCDDDQMSNDKERFARSDDEQSSADKERLARENHSEIERRRRNKMTAYITELSDMVPTCSALARKPDKLTILRMAVSHMKSLRGTGNTSTDGSYKPSFLTDQELKHLILEAADGFLFIVSCETGRVVYVSDSVTPVLNQPQSEWFGSTLYDQVHPDDVDKLREQLSTSENALTGRILDLKTGTVKKEGQQSSMRMCMGSRRSFICRMRCGSSSVDPVSVNRLSFVRNRCRNGLGSVKDGEPHFVVVHCTGYIKAWPPAGVSLPDDDPEAGQGSKFCLVAIGRLQVTSSPNCTDMSNVCQPTEFISRHNIEGIFTFVDHRCVATVGYQPQELLGKNIVEFCHPEDQQLLRDSFQQVVKLKGQVLSVMFRFRSKNQEWLWMRTSSFTFQNPYSDEIEYIICTNTNVKNSSQEPRPTLSNTIQRPQLGPTANLPLEMGSGQLAPRQQQQQTELDMVPGRDGLASYNHSQVVQPVTTTGPEHSKPLEKSDGLFAQDRDPRFSEIYHNINADQSKGISSSTVPATQQLFSQGNTFPPTPRPAENFRNSGLAPPVTIVQPSASAGQMLAQISRHSNPTQGATPTWTPTTRSGFSAQQVATQATAKTRTSQFGVGSFQTPSSFSSMSLPGAPTASPGAAAYPSLTNRGSNFAPETGQTAGQFQTRTAEGVGVWPQWQGQQPHHRSSSSEQHVQQPPAQQPGQPEVFQEMLSMLGDQSNSYNNEEFPDLTMFPPFSE

SEQ ID NO: 34 - protein, 328 aa, Homo sapiens
MAATTANPEMTSDVPSLGPAIASGNSGPGIQGGGAIVQRAIKRRPGLDFDDDGEGNSKFLRCDDDQMSNDKERFARSDDEQSSADKERLARENHSEIERRRRNKMTAYITELSDMVPTCSALARKPDKLTILRMAVSHMKSLRGTGNTSTDGSYKPSFLTDQELKHLILEAADGFLFIVSCETGRVVYVSDSVTPVLNQPQSEWFGSTLYDQVHPDDVDKLREQLSTSENALTGRILDLKTGTVKKEGQQSSMRMCMGSRRSFICRMRCGSSSVDPVSVNRLSFVRNRCRNGLGSVKDGEPHFVVVHCTGYIKAWPPAGVSLPDDDPA

SEQ ID NO: 35 - protein, 774 aa, Homo sapiens
MAATTANPEMTSDVPSLGPAIASGNSGPGIQGGGAIVQRAIKRRPGLDFDDDGEGNSKFLRCDDDQMSNDKERFARENHSEIERRRRNKMTAYITELSDMVPTCSALARKPDKLTILRMAVSHMKSLRGTGNTSTDGSYKPSFLTDQELKHLILEAADGFLFIVSCETGRVVYVSDSVTPVLNQPQSEWFGSTLYDQVHPDDVDKLREQLSTSENALTGRILDLKTGTVKKEGQQSSMRMCMGSRRSFICRMRCGSSSVDPVSVNRLSFVRNRCRNGLGSVKDGEPHFVVVHCTGYIKAWPPAGVSLPDDDPEAGQGSKFCLVAIGRLQVTSSPNCTDMSNVCQPTEFISRHNIEGIFTFVDHRCVATVGYQPQELLGKNIVEFCHPEDQQLLRDSFQQVVKLKGQVLSVMFRFRSKNQEWLWMRTSSFTFQNPYSDEIEYIICTNTNVKNSSQEPRPTLSNTIQRPQLGPTANLPLEMGSGQLAPRQQQQQTELDMVPGRDGLASYNHSQVVQPVTTTGPEHSKPLEKSDGLFAQDRDPRFSEIYHNINADQSKGISSSTVPATQQLFSQGNTFPPTPRPAENFRNSGLAPPVTIVQPSASAGQMLAQISRHSNPTQGATPTWTPTTRSGFSAQQVATQATAKTRTSQFGVGSFQTPSSFSSMSLPGAPTASPGAAAYPSLTNRGSNFAPETGQTAGQFQTRTAEGVGVWPQWQGQQPHHRSSSSEQHVQQPPAQQPGQPEVFQEMLSMLGDQSNSYNNEEFPDLTMFPPFSE

SEQ ID NO: 36 - protein, 870 aa, Homo sapiens
MTADKEKKRSSSERRKEKSRDAARCRRSKETEVFYELAHELPLPHSVSSHLDKASIMRLAISFLRTHKLLSSVCSENESEAEADQQMDNLYLKALEGFIAVVTQDGDMIFLSENISKFMGLTQVELTGHSIFDFTHPCDHEEIRENLSLKNGSGFGKKSKDMSTERDFFMRMKCTVTNRGRTVNLKSATWKVLHCTGQVKVYNNCPPHNSLCGYKEPLLSCLIIMCEPIQHPSHMDIPLDSKTFLSRHSMDMKFTYCDDRITELIGYHPEELLGRSAYEFYHALDSENMTKSHQNLCTKGQVVSGQYRMLAKHGGYVWLETQGTVIYNPRNLQPQCIMCVNYVLSEIEKNDVVFSMDQTESLFKPHLMAMNSIFDSSGKGAVSEKSNFLFTKLKEEPEELAQLAPTPGDAIISLDFGNQNFEESSAYGKAILPPSQPWATELRSHSTQSEAGSLPAFTVPQAAAPGSTTPSATSSSSSCSTPNSPEDYYTSLDNDLKIEVIEKLFAMDTEAKDQCSTQTDFNELDLETLAPYIPMDGEDFQLSPICPEERLLAENPQSTPQHCFSAMTNIFQPLAPVAPHSPFLLDKFQQQLESKKTEPEHRPMSSIFFDAGSKASLPPCCGQASTPLSSMGGRSNTQWPPDPPLHFGPTKWAVGDQRTEFLGAAPLGPPVSPPHVSTFKTRSAKGFGARGPDVLSPAMVALSNKLKLKRQLEYEEQAFQDLSGGDPPGGSTSHLMWKRMKNLRGGSCPLMPDKPLSANVPNDKFTQNPMRGLGHPLRHLPLPQPPSAISPGENSKSRFPPQCYATQYQDYSLSSAHKVSGMASRLLGPSFESYLLPELTRYDCEVNVPVLGSSTLLQGGDLLRALDQAT

(19) United States
(12) Patent Application Publication
(10) Pub. No.: US 2011/0166200 A1
(43) Pub. Date: Jul. 7, 2011

(54) METHODS OF USING MIR210 AS A BIOMARKER FOR HYPOXIA AND AS A THERAPEUTIC AGENT FOR TREATING CANCER

(76) Inventors: Zhan Zhang, Kirkland, WA (US); Sun Hong, Needham, MA (US); Hongyue Dai, Chestnut Hill, MA (US); Jan Schelter, Bellevue, WA (US); Julia Burchard, San Francisco, CA (US); Xudong Dai, Kirkland, WA (US); Michael Carleton, Bothell, WA (US); Michael Cleary, Jamison, PA (US); Peter Linsley, Settle, WA (US); Carla Grandori, Seattle, WA (US)

(21) Appl. No.: 12/989,198

(22) PCT Filed: Apr. 16, 2009
(86) PCT No.: PCT/US2009/040788

§ 371 Date: Mar. 10, 2011

(30) Foreign Application Priority Data
Apr. 24, 2008 (US) 61047690

(51) Int. Cl. A61K 31/713; C12Q 1/68; C12N 5/09; A61P 35/00

(52) U.S. Cl. 514/44 A; 435/6.1; 435/375

(57) ABSTRACT

The present invention provides compositions and methods for predicting the hypoxia response in tumor cells, methods for predicting the likelihood of cancer metastasis, and methods for inhibiting tumor cell proliferation using a microRNA comprising miR-210.

### FIELD OF THE INVENTION

The invention relates to the use of miR-210 as a biomarker for hypoxia in tumor cells, and as a therapeutic agent for inhibiting growth of tumor cells.

### BACKGROUND

Intratumoral hypoxia is a hallmark of most solid tumors and results from increased oxygen consumption and/or insufficient blood supply. Many of the hypoxia induced cellular responses are mediated through the hypoxia-inducible factors (HIFs) (Pouyssegur, J., et al., “Hypoxia Signalling in Cancer and Approaches to Enforce Tumour Regression,” Nature 441:437-443, 2006; Semenza, G. L., “Targeting HIF-1 for Cancer Therapy,” Nat. Rev. Cancer 3:721-723, 2003), which act to regulate expression of genes involved in angiogenesis, survival, cell metabolism, invasion and other functions (Keith, B., and M. C. Simon, “Hypoxia-Inducible Factors, Stem Cells, and Cancer,” Cell 129:465-472, 2007). HIFs are members of the basic-helix-loop-helix-Per-Arnt-Sim domain (PAS) protein family of transcription factors that bind to hypoxia regulated elements (HREs) in the promoter or enhancer regions of a specific set of target genes. HIFs function as obligate heterodimers composed of an α-subunit (HIF-1α or HIF-2α) and β-subunit (HIF-1β). In the presence of oxygen, the α-subunits are hydroxylated at two key proline residues in their oxygen-dependent degradation domain (ODD) by a family of prolyl hydroylases. Hydroxylated HIF-α protein is then recognized by the tumor suppressor Von Hippel-Lindau (VHL), part of an E3 ubiquitin ligase complex, ubiquitinated, and targeted for proteosomal degradation (Kaelin, W. G., Jr., “The von Hippel-Lindau Protein, HIF Hydroxylation, and Oxygen Sensing,” Biochem. Biophys. Res. Commun. 338:627-638, 2005; Semenza, G. L., “Hypoxia and Cancer,” Cancer Metastasis Rev. 26:223-224, 2007; Shivdasani, R. A., “microRNAs: Regulators of Gene Expression and Cell Differentiation,” Blood 108:3646-3653, 2006; Wang, G. L., et al., “Hypoxia-Inducible Factor 1 is a Basic-Helix-Loop-Helix-PAS Heterodimer Regulated by Cellular 02 Tension,” Proc. Natl. Acad. Sci. USA 92:5510-5514, 1995; Wang, G. L., and G. L. Semenza, “General Involvement of Hypoxia-Inducible Factor 1 in Transcriptional Response to Hypoxia,” Proc. Natl. Acad. Sci. USA 90:4304-4308, 1993; Wang, G. L., and G. L. Semenza, “Purification and Characterization of Hypoxia-Inducible Factor 1,” J. Biol. Chem. 270:1230-1237, 1995). Under hypoxic conditions, HIF-α protein is not degraded and translocates to the nucleus where it binds to the constitutively expressed HIF-1β and activates HIF target genes.

The two HIF α-subunits are differentially expressed, with HIF-1α being more ubiquitous while HIF-2α expression is limited to specific tissues, including kidney, heart, lungs, and endothelium. In addition, HIF-1α and HIF-2α are functionally distinct, as evidenced by both gene knock-out studies in mice and by their ability to either promote (HIF-2α) or inhibit (HIF-1α) VHL deficient renal tumor cell proliferation (Kondo, K., et al., “Inhibition of HIF2Alpha is Sufficient to Suppress pVHL-Defective Tumor Growth,” PLoS Biol. 1:E83, 2003; Kondo, K., et al., “Inhibition of HIF is Necessary for Tumor Suppression by the von Hippel-Lindau Protein,” Cancer Cell 1:237-246, 2002; Maranchie, J. K., et al., “The Contribution of VHL Substrate Binding and HIF1-Alpha to the Phenotype of VHL Loss in Renal Cell Carcinoma,” Cancer Cell 1:247-255, 2002; Raval, R. R., et al., “Contrasting Properties of Hypoxia-Inducible Factor 1 (HIF-1) and HIF-2 in von Hippel-Lindau-Associated Renal Cell Carcinoma,” Mol. Cell. Biol. 25:5675-5686, 2005). Although HIF-1α and HIF-2α regulate unique target genes (e.g., HIF-1α activates genes involved in glycolysis and HIF-2α induces stem cell factor Oct4, TGFα, lysyl oxidase and cyclinD1), they also share common targets, such as VEGF and ADRP (adipose differentiation-related protein) (Gordan, J. D., and M. C. Simon, “Hypoxia-Inducible Factors: Central Regulators of the Tumor Phenotype,” Curr. Opin. Genet. Dev. 17:71-77, 2007; Gordan, J. D., et al., “HIF and c-Myc: Sibling Rivals for Control of Cancer Cell Metabolism and Proliferation,” Cancer Cell 12:108-113, 2007b). HIF-1α and HIF-2α also differ as they exert opposite effects on the activity of the c-Myc oncoprotein. Stabilization of HIF-1α by hypoxia leads to cell cycle arrest at G1/S by inhibition of c-Myc transcriptional activity through multiple mechanisms involving both direct binding to c-Myc as well as through activation of the antagonist MXI-1 (Dang, C. V., et al., “The Interplay Between MYC and HIF in Cancer,” Nat. Rev. Cancer, 2007; Gordan, J. D., et al., “HIF-2Alpha Promotes Hypoxic Cell Proliferation by Enhancing c-Myc Transcriptional Activity,” Cancer Cell 11:335-347, 2007a; Gordan, J. D., and M. C. Simon, “Hypoxia-Inducible Factors: Central Regulators of the Tumor Phenotype,” Curr. Opin. Genet. Dev. 17:71-77, 2007; Gordan, J. D., et al., “HIF and c-Myc: Sibling Rivals for Control of Cancer Cell Metabolism and Proliferation,” Cancer Cell 12:108-113, 2007b; Zhang, H., et al., “HIF-1 Inhibits Mitochondrial Biogenesis and Cellular Respiration in VHL-Deficient Renal Cell Carcinoma by Repression of c-Myc Activity,” Cancer Cell 11:407-420, 2007). In contrast to HIF-1α, HIF-2α promotes cell cycle progression by enhancing c-Myc activity through binding and stabilization of complexes between Myc and its binding partner, Max (Gordan, J. D., et al., “HIF-2Alpha Promotes Hypoxic Cell Proliferation by Enhancing c-Myc Transcriptional Activity,” Cancer Cell 11:335-347, 2007a; Gordan, J. D., et al., “HIF and c-Myc: Sibling Rivals for Control of Cancer Cell Metabolism and Proliferation,” Cancer Cell 12:108-113, 2007b). Recent data also indicates Myc-induced lymphomagenesis requires HIF-1α, implying that the Myc and HIF pathway interaction is complex and reciprocal (Dang, C. V., et al., “The Interplay Between MYC and HIF in Cancer,” Nat. Rev. Cancer, 2007).

Hypoxia alters the expression of hundreds of mRNAs that are essential for many aspects of tumorigenesis and the HIF transcription factors play a central role in this response (Chi, J. T., et al., “Gene Expression Programs in Response to Hypoxia: Cell Type Specificity and Prognostic Significance in Human Cancers,” PLoS Med. 3:e47, 2006; Gordan, J. D., and M. C. Simon, “Hypoxia-Inducible Factors: Central Regulators of the Tumor Phenotype,” Curr. Opin. Genet. Dev. 17:71-77, 2007). Recently, the effect of hypoxia on microRNA expression has been reported (Donker, R. B., et al., “The Expression of Argonaute2 and Related microRNA Biogenesis Proteins in Normal and Hypoxic Trophoblasts,” Mol. Hum. Reprod. 13:273-279, 2007; Fabbri et al., “Regulatory Mechanisms of microRNAs Involvement in Cancer,” Expert Opin. Biol. Ther. 7:1009-1019, 2007; Huam et al., “miRNA-Directed Regulation of VEGF and Other Angiogenic Factors Under Hypoxia,” PLoS ONE 1:e116, 2006; Kulshreshtha et al., “Regulation of microRNA Expression: The Hypoxic Component,” Cell Cycle 6:1426-1431, 2007a; Kulshreshtha et al., “A microRNA Signature of Hypoxia,” Mol. Cell. Biol. 27:1859-1867, 2007b). microRNAs are a novel class of gene regulators that can each regulate as many as several hundred genes with spatial and temporal specificity (Bushati, N., and S. M. Cohen, “microRNA Functions,” Annu. Rev. Cell Dev. Biol. 23:175-205, 2007; Carleton et al., “microRNAs and Cell Cycle Regulation,” Cell Cycle 6:2127-2132, 2007; Dalmay, T., and D. R. Edwards, “microRNAs and the Hallmarks of Cancer,” Oncogene 25:6170-6175, 2006). microRNAs have been proposed to contribute to oncogenesis by functioning either as tumor suppressors or oncogenes (Esquela-Kerscher, A., and F. J. Slack, “Oncomirs—microRNAs With a Role in Cancer,” Nat. Rev. Cancer 6:259-269, 2006; Fabbri et al., 2007; Leung, A. K., and P. A. Sharp, “microRNAs: A Safeguard Against Turmoil?” Cell 130:581-585, 2007; Shivdasani, R. A., “microRNAs: Regulators of Gene Expression and Cell Differentiation,” Blood 108:3646-3653, 2006; Stahlhut Espinosa, C. E., and F. J. Slack, “The Role of microRNAs in Cancer,” Yale J. Biol. Med. 79:131-140, 2006).

Given the importance of hypoxia in tumorigenesis and metastasis, there is a need to identify modulators of the hypoxia response pathway. There is also a need to identify biomarkers that are predictive of patient outcomes after tumor diagnosis.

### SUMMARY

In one aspect, a method is provided for determining a hypoxic state in tumor cells obtained from a subject. The method comprises (a) measuring the level of miR-210 in tumor cells, and (b) comparing the level of miR-210 with a hypoxia reference value, wherein a level greater than the hypoxia reference value is indicative of a hypoxic state in the tumor cells.

In another aspect, a method is provided for predicting the likelihood of metastasis of a tumor in a subject. The method according to this aspect of the invention comprises (a) measuring the level of miR-210 in tumor cells obtained from a tumor in a subject, and (b) comparing the measured level of miR-210 with a metastasis reference value, wherein a level or miR-210 equal to or greater than the metastasis reference value is predictive of metastasis of the tumor in the subject.

In another aspect, a method is provided for inhibiting tumor cell proliferation. The method according to this aspect of the invention comprises (a) measuring the level of Myc protein or nucleic acid in a tumor cell sample; (b) comparing the measured level of Myc with a Myc reference value; and (c) contacting the tumor cells having a level of Myc equal to or greater than the Myc reference value with an amount of an siNA comprising miR-210 effective to inhibit the proliferation of tumor cells.

In another aspect, a method is provided for reducing the tumor burden in a subject. The method according to this aspect of the invention comprises contacting a plurality of tumor cells with an amount of a small interfering nucleic acid (siNA) effective to reduce tumor burden in the subject, wherein said siNA comprises a guide strand contiguous nucleotide sequence of at least 18 nucleotides, wherein said guide strand comprises a seed region consisting of nucleotide positions 1 to 12, wherein position 1 represents the 5′-end of said guide strand and wherein said seed region comprises a nucleotide sequence of at least 6 contiguous nucleotides that is identical to 6 contiguous nucleotides of SEQ ID NO:4.

In another aspect, a method is provided for inhibiting the proliferation of tumor cells. The methods according to this aspect of the invention comprise (a) measuring the level of Myc protein or nucleic acid in the tumor cells; (b) comparing the measured level of Myc in the tumor cells with a Myc reference value; and (c) contacting the tumor cells having a level of Myc equal to or greater than the Myc reference value with an amount of an inhibitor of the expression or activity of (i) a polypeptide having at least 95% identity to the full length polypeptide set forth in SEQ ID NO:30; or (ii) a polynucleotide having at least 95% identity to the full length polynucleotide set forth in SEQ ID NO:29; effective to inhibit proliferation of the tumor cells.

In yet another aspect, a method is provided for inhibiting tumor cell proliferation in a subject. The method according to this aspect of the invention comprises (a) measuring the level of miR-210 in tumor cells from the subject; (b) comparing the measured level of miR-210 with a hypoxia reference value; wherein measured levels equal to or greater than the hypoxia reference value indicate the tumor cells are hypoxic; and (c) contacting the tumor cells with an inhibitor of the hypoxia response pathway; thereby inhibiting the proliferation of tumor cells in the subject.

In a further aspect, a method is provided for inhibiting tumor cell proliferation in a subject. The methods according to this aspect of the invention comprise (a) measuring the level of miR-210 in tumor cells from the subject; (b) comparing the measured level of miR-210 with a hypoxia reference value, wherein measured levels equal to or greater than the hypoxia reference value indicate the tumor cells are hypoxic; and (c) contacting the tumor cells with a miR-210 inhibitor, thereby inhibiting the proliferation of tumor cells in the subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows that miR-210 is up-regulated by hypoxia in HT29 colon cancer cells exposed to normal conditions (21% O₂) or hypoxic conditions (1% O₂) as described in Example 1;

FIG. 2 shows that miR-210 upregulation by hypoxia in HCT116 Dicer^{ex5} cells is reduced by siRNA to HIF-1β and HIF-1α as described in Example 2;

FIG. 3A shows that HIF-1α protein binds to the miR-210 promoter under hypoxic conditions in HuH7 human hepatoma cells as described in Example 2;

FIG. 3B shows that HIF-1α protein binds to the miR-210 promoter under hypoxic conditions in U251 glioma cells as described in Example 2;

FIG. 4A shows that pri-miR-210 is overexpressed in human kidney tumors compared to adjacent normal tissues as described in Example 3;

FIG. 4B shows that pri-miR-210 is overexpressed in human lung tumors compared to adjacent normal tissues as described in Example 3;

FIG. 4C shows that pri-miR-210 is overexpressed in human breast tumors compared to adjacent normal tissues as described in Example 3;

FIG. 5 shows that miR-210 expression positively correlates with genes up-regulated by hypoxia in human tumors as described in Example 3;

FIG. 6A shows that overexpression of pri-miR-210 in breast cancer tumors is positively correlated with the probability of metastasis of breast cancer cells as described in Example 3;

FIG. 6B shows that overexpression of pri-miR-210 in melanoma cancer cells is positively correlated with the probability of metastasis of melanoma cancer cells as described in Example 3;

FIG. 7A shows that the introduction of miR-210 or Mnt siRNA into human foreskin fibroblasts (HFFs) that overexpress c-Myc results in a decrease in the number of live cells as described in Example 7;

FIG. 7B shows that the introduction of miR-210 or Mnt siRNA into human foreskin fibroblasts that are transduced with an empty vector (pBABE), and therefore do not overexpress c-Myc, does not result in a decrease in the number of live cells as described in Example 7;

FIG. 7C shows that the introduction of miR-210 or Mnt siRNA into human foreskin fibroblasts that overexpress c-Myc results in an increase in the percentage of dead cells as described in Example 7;

FIG. 7D shows that the introduction of miR-210 or Mnt siRNA into human foreskin fibroblasts that are transduced with an empty vector (pBABE), and therefore do not overexpress c-Myc, does not result in an increase in the percentage of dead cells as described in Example 7;

FIG. 8 shows a proposed model illustrating the intersection of the hypoxia response, miR-210 and c-Myc pathways.

### DETAILED DESCRIPTION

This section presents a detailed description of the many different aspects and embodiments that are representative of the inventions disclosed herein. This description is by way of several exemplary illustrations, of varying detail and specificity. Other features and advantages of these embodiments are apparent from the additional descriptions provided herein, including the different examples. The provided examples illustrate different components and methodology useful in practicing various embodiments of the invention. The examples are not intended to limit the claimed invention. Based on the present disclosure the ordinary skilled artisan can identify and employ other components and methodology useful for practicing the present invention.

### I. DEFINITIONS

Unless defined otherwise, all technical and scientific terms used herein have the meaning commonly understood by one of ordinary skill in the art to which this invention belongs. Practitioners are particularly directed to Sambrook et al., Molecular Cloning: A Laboratory Manual, 2d ed., Cold Spring Harbor Press, Plainsview, N.Y. (1989), and Ausubel et al., Current Protocols in Molecular Biology (Supplement 47), John Wiley & Sons, New York (1999), for definitions and terms of the art.

It is contemplated that the use of the term “about” in the context of the present invention is to connote inherent problems with precise measurement of a specific element, characteristic, or other trait. Thus, the term “about,” as used herein in the context of the claimed invention, simply refers to an amount or measurement that takes into account single or collective calibration and other standardized errors generally associated with determining that amount or measurement. Thus, any measurement or amount referred to in this application can be used with the term “about” if that measurement or amount is susceptible to errors associated with calibration or measuring equipment, such as a scale, pipetteman, pipette, graduated cylinder, etc.

The use of the word “a” or “an” when used in conjunction with the term “comprising” in the claims and/or the specification may mean “one,” but it is also consistent with the meaning of “one or more,” “at least one,” and “one or more than one.”The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

It is contemplated that any embodiment discussed in this specification can be implemented with respect to any method, kit, reagent, or composition of the invention and vice versa. Furthermore, compositions of the invention can be used to achieve methods of the invention.

As used herein, the term “gene” encompasses the meaning known to one of skill in the art, i.e., a nucleic acid (e.g., DNA or RNA) sequence that comprises coding sequences necessary for the production of an RNA and/or a polypeptide or its precursor, as well as noncoding sequences (untranslated regions) surrounding the 5′- and 3′-ends of the coding sequences. The sequences that are located 5′ of the coding region and that are present on the mRNA are referred to as 5′-untranslated sequences (“5′UTR”). The sequences that are located 3′ or downstream of the coding region and that are present on the mRNA are referred to as 3′-untranslated sequences, or (“3′UTR”). The term “gene” may include gene regulatory sequences (e.g., promoters, enhancers, etc.) and/or intron sequences. The term “gene” encompasses RNA, cDNA, and genomic forms of a gene. The term “gene” also encompasses nucleic acid sequences that comprise microRNAs and other non-protein encoding sequences including, for example, transfer RNAs, ribosomal RNAs, etc. For clarity, the term gene generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid. In some cases, the gene includes regulatory sequences involved in transcription or message production or composition. In other embodiments, the gene comprises transcribed sequences that encode for a protein, polypeptide or peptide. A functional polypeptide can be encoded by a full length coding sequence or by any portion of the coding sequence as long as the desired activity or functional properties (e.g., enzymatic activity, ligand binding, signal transduction, antigenic presentation) of the polypeptide are retained.

In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed and the complement thereof. In particular embodiments, the transcribed nucleotide sequence comprises at least one functional protein, polypeptide, and/or peptide encoding unit. As will be understood by those in the art, this functional term “gene” includes both genomic sequences, RNA or cDNA sequences, or smaller engineered nucleic acid segments including nucleic acid segments of a non-transcribed part of a gene including, but not limited to, the non-transcribed promoter or enhancer regions of a gene. Smaller engineered gene nucleic acid segments may express or may be adapted to express using nucleic acid manipulation technology, proteins, polypeptides, domains, peptides, fusion proteins, mutants, and/or such like.

The term “gene expression,” as used herein, refers to the process of transcription and translation of a gene to produce a gene product, be it RNA or protein. Thus, modulation of gene expression may occur at any one or more of many levels including transcription, post-transcriptional processing, translation, post-translational modification, and the like.

As used herein, the term “expression cassette” refers to a nucleic acid molecule that comprises at least one nucleic acid sequence that is to be expressed, along with its transcription and translational control sequences. The expression cassette typically includes restriction sites engineered to be present at the 5′- and 3′-ends such that the cassette can be easily inserted, removed, or replaced in a gene delivery vector. Changing the cassette will cause the gene delivery vector into which it is incorporated to direct the expression of a different sequence.

As used herein, the terms “microRNA species,” “microRNA,” “miRNA,” or “mi-R” refer to small, non-protein coding RNA molecules that are expressed in a diverse array of eukaryotes, including mammals. MicroRNA molecules typically have a length in the range of from 15 to 120 nucleotides, the size depending upon the specific microRNA species and the degree of intracellular processing. Mature, fully processed miRNAs are about 15 to 30, 15 to 25, or 20 to 30 nucleotides in length and, more often, between about 16 to 24, 17 to 23, 18 to 22, 19 to 21, or 21 to 24 nucleotides in length. MicroRNAs include processed sequences as well as corresponding long primary transcripts (pri-miRNAs) and processed stem-loop precursors (pre-miRNAs). Some microRNA molecules function in living cells to regulate gene expression via RNA interference. A representative set of microRNA species is described in the publicly available miRBase sequence database as described in Griffith-Jones et al., Nucleic Acids Research 32:D109-D111 (2004), and Griffith-Jones et al., Nucleic Acids Research 34:D140-D144 (2006), accessible on the World Wide Web at the Welcome Trust Sanger Institute Web site.

As used herein, “miR-210” refers to the mature miR210 microRNA (SEQ ID NO:1). The primary transcript is referred to as pri-miR-210 (SEQ ID NO:2) (Genbank Accession number: AK123483). The stem-loop precursor is referred to as pre-miR-210 (SEQ ID NO:3) (Sanger microRNA database accession number: hsa-mir-210 MI0000286).

As used herein, the term “microRNA family” refers to a group of microRNA species that share identity across at least 6 consecutive nucleotides within nucleotide positions 1 to 12 of the 5′-end of the microRNA molecule, also referred to as the “seed region,” as described in Brennecke, J., et al., PloS Biol. 3(3):pe85 (2005). As used herein, the seed region of miR-210 corresponds to SEQ ID NO:4.

As used herein, the term “microRNA family member” refers to a microRNA species that is a member of a microRNA family.

As used herein, the term “microRNA inhibitor” refers to a nucleic acid molecule that inhibits the function of a microRNA. For example, the inhibitor may be a single-stranded oligonucleotide that binds to a mature microRNA by Watson-Crick base pairing, such as sense-antisense pairing. The antisense oligonucleotide may comprise chemically modified nucleic acids, such as locked nucleic acid (LNA) nucleosides and 2′-O-methyl sugar modified RNA. Other examples of microRNA inhibitors include antagomirs, RNA-like oligonucleotides comprising complete 2′-O-methylation of sugar, a phosphorothioate backbone, and a cholesterol-moiety at the 3′-end (Krutzfeldt, J., et al., Nucleic Acids Res. 35:25885-2892, 2007), and antisense oligonucleotides with a complete 2′-O-methoxyethyl and phosphorothioate modification (Esau, C., et al., Cell. Metab. 3:87-98, 2006). Other examples of microRNA inhibitors include microRNA sponges comprising transcripts with multiple copies of a microRNA binding site located in the 3′ UTR, as described in Ebert, M. S., et al., Nature Methods 4:721-726, 2007. MicroRNA inhibitors are also commercially available, for example, from Exiqon A/S (Denmark); Ambion, Inc. (Austin, Tex.); and Dharmacon, Inc. (Lafayette, Colo.). Representative non-limiting examples of microRNA inhibitors are described in Example 4.

As used herein, the term “RNA interference” or “RNAi” refers to the silencing, inhibition, or reduction of gene expression by iRNA (“interfering RNA”) agents (e.g., siRNAs, miRNAs, shRNAs) via the process of sequence-specific, post-transcriptional gene silencing in animals and plants, initiated by an iRNA agent that has a seed region sequence in the iRNA guide strand that is complementary to a sequence of the silenced gene.

As used herein, the term an “iNA agent” (abbreviation for “interfering nucleic acid agent”), refers to a nucleic acid agent, for example, RNA or chemically modified RNA, which can down-regulate the expression of a target gene. While not wishing to be bound by theory, an iNA agent may act by one or more of a number of mechanisms, including post-transcriptional cleavage of a target mRNA, or pre-transcriptional or pre-translational mechanisms. An iNA agent can include a single strand (ss) or can include more than one strands, e.g., it can be a double-stranded (ds) iNA agent.

As used herein, the term “single strand iRNA agent” or “ssRNA” is an iRNA agent that consists of a single molecule. It may include a duplexed region, formed by intra-strand pairing, e.g., it may be or include a hairpin or panhandle structure. The ssRNA agents of the present invention include transcripts that adopt stem-loop structures, such as shRNA, that are processed into a double stranded siRNA.

As used herein, the term “dsiNA agent” is a dsNA (double stranded nucleic acid (NA)) agent that includes two strands that are not covalently linked, in which interchain hybridization can form a region of duplex structure. The dsNA agents of the present invention include silencing dsNA molecules that are sufficiently short that they do not trigger the interferon response in mammalian cells.

As used herein, the term “siRNA” refers to a small interfering RNA. In some embodiments, siRNA includes the term microRNA. In other embodiments, siRNA include short interfering RNA of about 15 to 60, 15 to 50, 15 to 50, or 15 to 40 (duplex) nucleotides in length, more typically about 15 to 30, 15 to 25, or 19 to 25 (duplex) nucleotides in length and is preferably about 20 to 24 or about 21 to 22 or 21 to 23 (duplex) nucleotides in length (e.g., each complementary sequence of the double stranded siRNA is 15 to 60, 15 to 50, 15 to 50, 15 to 40, 15 to 30, 15 to 25, or 19 to 25 nucleotides in length, preferably about 20 to 24 or about 21 to 22 or 21 to 23 nucleotides in length, preferably 19 to 21 nucleotides in length, and the double stranded siRNA is about 15 to 60, 15 to 50, 15 to 50, 15 to 40, 15 to 30, 15 to 25, or 19 to 25, preferably about 20 to 24 or about 21 to 22 or 19 to 21 or 21 to 23 base pairs in length). siRNA duplexes may comprise 3′-overhangs of about 1 to about 4 nucleotides, preferably of about 2 to about 3 nucleotides and 5′-phosphate termini. In some embodiments, the siRNA lacks a terminal phosphate.

Non-limiting examples of siRNA molecules of the invention may include a double-stranded polynucleotide molecule comprising self-complementary sense and antisense regions, wherein the antisense region comprises nucleotide sequence that is complementary to nucleotide sequence in a target nucleic acid molecule or a portion thereof (alternatively referred to as the guide region or guide strand when the molecule contains two separate strands) and the sense region having nucleotide sequence corresponding to the target nucleic acid sequence or a portion thereof (also referred as the passenger region or the passenger strand when the molecule contains two separate strands). The siRNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure, for example, wherein the double stranded region is about 18 to about 30, e.g., about 18, 19, 20, 21, 22, 23, 24, 25, 26, 27, 28, 29, or 30 base pairs); the antisense strand (guide strand) comprises nucleotide sequence that is complementary to nucleotide sequence in a target nucleic acid molecule or a portion thereof and the sense strand (passenger strand) comprises nucleotide sequence corresponding to the target nucleic acid sequence or a portion thereof (e.g., about 15 to about 25 nucleotides of the siRNA molecule are complementary to the target nucleic acid or a portion thereof). Typically, a short interfering RNA (siRNA) refers to a double-stranded RNA molecule of about 17 to about 29 base pairs in length, preferably from 19 to 21 base pairs, one strand of that is complementary to a target mRNA, that when added to a cell having the target mRNA or produced in the cell in vivo, causes degradation of the target mRNA. Preferably the siRNA is perfectly complementary to the target mRNA, but it may have one or two mismatched base pairs. Table 1 shows the nucleotide sequences of the guide strands of miRNAs and siRNAs of the invention.

TABLE 1
SEQ ID NOs: of miRNAs and siRNAs

| | | SEQ |
|---|---|---|
| | Guide Strand Sequence | ID |
| Identifier | (5′ to 3′) | NO: |
| miR-210 | CUGUGCGUGUGACAGCGGCUG | 1 |
| miR-210 | CUGUGCGUGUGA | 4 |
| seed region | | |
| miR-210 mt | CUGUCGGUGUGACAGCGGCUG | 5 |
| HIF-1α siRNA1 | GUCCUUAAACCGGUUGAAUdTdT | 6 |
| HIF-1α siRNA2 | GCAACUUGAGGAAGUACCAdTdT | 7 |
| HIF-1α siRNA3 | CCUAAUAGUCCCAGUGAAUdTdT | 8 |
| HIF-1β siRNA1 | GGCUCAAGGAGAUCGUUUAdTdT | 9 |
| HIF-1β siRNA2 | GAAUGGACUUGGCUCUGUAdTdT | 10 |
| HIF-1β siRNA3 | GCCACAGUCUGAAUGGUUUdTdT | 11 |
| HIF-2α siRNA1 | CGGGCCAGGUGAAAGUCUAdTdT | 12 |
| HIF-2α siRNA2 | GCGACAGCUGGAGUAUGAAdTdT | 13 |
| HIF-2α siRNA3 | GCUUCAGUGCCAUGACAAAdTdT | 14 |
| Mnt siRNA1 | CGUCCAAUCUGAGCGUGCUTT | 15 |
| Mnt siRNA2 | GCUGGCACGUGAGAAGAUUTT | 16 |
| Mnt siRNA3 | GGUACAUCCAGUCCCUGAATT | 17 |
| Myc siRNA1 | GCUUGUACCUGCAGGAUCUTT | 18 |
| Myc siRNA2 | CGAUGUUGUUUCUGUGGAATT | 19 |
| Myc siRNA3 | CGAGAACAGUUGAAACACATT | 20 |

Alternatively, the siRNA is assembled from a single oligonucleotide, where the self-complementary sense and antisense regions of the siRNA are linked by means of a nucleic acid based or non-nucleic acid-based linker(s). The siRNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siRNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNAi. The siRNA can also comprise a single stranded polynucleotide having nucleotide sequence complementary to nucleotide sequence in a target nucleic acid molecule or a portion thereof (for example, where such siRNA molecule does not require the presence within the siRNA molecule of nucleotide sequence corresponding to the target nucleic acid sequence or a portion thereof), wherein the single stranded polynucleotide can further comprise a terminal phosphate group, such as a 5′-phosphate (see, for example, Martinez et al., Cell 110:563-574, 2002; and Schwarz et al., Molecular Cell 10:537-568, 2002), or 5′,3′-diphosphate. In certain embodiments, the siRNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der Waals interactions, hydrophobic interactions, and/or stacking interactions. In certain embodiments, the siRNA molecules of the invention comprise nucleotide sequence that is complementary to nucleotide sequence of a target gene. In another embodiment, the siRNA molecule of the invention interacts with nucleotide sequence of a target gene in a manner that causes inhibition of expression of the target gene.

As used herein, the miRNA and siRNA molecules need not be limited to those molecules containing only RNA but may further encompasses chemically-modified nucleotides and non-nucleotides. For example, International PCT Publications No. WO 2005/078097, WO 2005/0020521, and WO 2003/070918 detail various chemical modifications to RNAi molecules, wherein the contents of each reference are incorporated by reference in its entirety. In certain embodiments for example, the short interfering nucleic acid molecules may lack 2′-hydroxy (2′-OH) containing nucleotides. The siRNA can be chemically synthesized or may be encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., PNAS USA 99:9942-7, 2002; Calegari et al., PNAS USA 99:14236, 2002; Byrom et al., Ambion TechNotes 10(1):4-6, 2003; Kawasaki et al., Nucleic Acids Res. 31:981-7, 2003; Knight and Bass, Science 293:2269-71, 2001; and Robertson et al., J. Biol. Chem. 243:82, 1968). The long dsRNA can encode for an entire gene transcript or a partial gene transcript.

As used herein, “percent modification” refers to the number of nucleotides in each of the strand of the siRNA or to the collective dsRNA that have been modified. Thus, 19% modification of the antisense strand refers to the modification of up to 4 nucleotides/bp in a 21 nucleotide sequence (21 mer). One hundred percent (100%) refers to a fully modified dsRNA. The extent of chemical modification will depend upon various factors well known to one skilled in the art such as, for example, target mRNA, off-target silencing, degree of endonuclease degradation, etc.

As used herein, the term “shRNA” or “short hairpin RNAs” refers to an RNA molecule that forms a stem-loop structure in physiological conditions, with a double-stranded stem of about 17 to about 29 base pairs in length, where one strand of the base-paired stem is complementary to the mRNA of a target gene. The loop of the shRNA stem-loop structure may be any suitable length that allows inactivation of the target gene in vivo. While the loop may be from 3 to 30 nucleotides in length, typically it is 1 to 10 nucleotides in length. The base paired stem may be perfectly base paired or may have 1 or 2 mismatched base pairs. The duplex portion may, but typically does not, contain one or more bulges consisting of one or more unpaired nucleotides. The shRNA may have non-base-paired 5′- and 3′-sequences extending from the base-paired stem. Typically, however, there is no 5′-extension. The first nucleotide of the shRNA at the 5′-end is a G, because this is the first nucleotide transcribed by polymerase III. If G is not present as the first base in the target sequence, a G may be added before the specific target sequence. The 5′G typically forms a portion of the base-paired stem. Typically, the 3′-end of the shRNA is a poly U segment that is a transcription termination signal and does not form a base-paired structure. As described in the application and known to one skilled in the art, shRNAs are processed into siRNAs by the conserved cellular RNAi machinery. Thus shRNAs are precursors of siRNAs and are, in general, similarly capable of inhibiting expression of a target mRNA transcript. For the purpose of description, in certain embodiments, the shRNA constructs of the invention target one or more mRNAs that are targeted by miR-210. The strand of the shRNA that is antisense to the target gene transcript is also known as the “guide strand.”

As used herein, the term “microRNA responsive target site” refers to a nucleic acid sequence ranging in size from about 5 to about 25 nucleotides (such as 5, 6, 7, 8, 9, 10, 11, 12, 13, 14, 15, 16, 17, 18, 19, 20, 21, 22, 23, 24, or 25 nucleotides) that is complementary, or essentially complementary, to at least a portion of a microRNA molecule. In some embodiments, the microRNA responsive target site comprises at least 6 consecutive nucleotides, at least 7 consecutive nucleotides, at least 8 consecutive nucleotides, or at least 9 nucleotides that are complementary to the seed region of a microRNA molecule (i.e., within nucleotide positions 1 to 12 of the 5′-end of the microRNA molecule, referred to as the “seed region.”

The phrase “inhibiting expression of a target gene” refers to the ability of an RNAi agent such as a microRNA or an siRNA to silence, reduce, or inhibit expression of a target gene. Said another way, to “inhibit,” “down-regulate,” or “reduce,” is meant that the expression of the gene, or level of RNA molecules or equivalent RNA molecules encoding one or more proteins or protein subunits, or activity of one or more proteins or protein subunits, is reduced below that observed in the absence of the RNAi agent. For example, an embodiment of the invention comprises introduction of a miR-210-like siRNA molecule into cells to inhibit, down-regulate, or reduce expression of one or more genes regulated by miR-210 as compared to the level observed for the miR-210 regulated gene in a control cell to which a miR-210-like siRNA molecule has not been introduced. As used herein, the term “miR-210-like” siRNA refers to a siRNA that shares at least 6 consecutive nucleotides within nucleotide positions 1 to 12 of SEQ ID NO:1. In another embodiment, inhibition, down-regulation, or reduction contemplates inhibition of the target miR-210 responsive genes below the level observed in the presence of, for example, a miR210-like siRNA molecule with scrambled sequence or with mismatches. In yet another embodiment, inhibition, down-regulation, or reduction of gene expression with a miR210-like siRNA molecule of the instant invention is greater in the presence of the invention miR210-like siRNA (e.g., siRNA that down regulates one or more miR-210 pathway gene mRNA levels), than in its absence. In one embodiment, inhibition, down regulation, or reduction of gene expression is associated with post transcriptional silencing, such as RNAi mediated cleavage of a target nucleic acid molecule (e.g., RNA) or inhibition of translation.

To examine the extent of gene silencing, a test sample (e.g., a biological sample from organism of interest expressing the target gene(s) or a sample of cells in culture expressing the target gene(s)) is contacted with an siRNA that silences, reduces, or inhibits expression of the target gene(s). Expression of the target gene in the test sample is compared to expression of the target gene in a control sample (e.g., a biological sample from organism of interest expressing the target gene or a sample of cells in culture expressing the target gene) that is not contacted with the siRNA. Control samples (i.e., samples expressing the target gene) are assigned a value of 100%. Silencing, inhibition, or reduction of expression of a target gene is achieved when the value of test the test sample relative to the control sample is about 95%, 90%, 85%, 80%, 75%, 70%, 65%, 60%, 55%, 50%, 45%, 40%, 35%, 30%, 25%, 20%, or 10%. Suitable assays include, e.g., examination of protein or mRNA levels using techniques known to those of skill in the art such as dot blots, northern blots, in situ hybridization, ELISA, microarray hybridization, immunoprecipitation, enzyme function, as well as phenotypic assays known to those of skill in the art.

An “effective amount” or “therapeutically effective amount” of an siRNA or an RNAi agent is an amount sufficient to produce the desired effect. In one embodiment of the methods of the invention, an effective amount is an amount sufficient to produce the effect of inhibition of expression of a target sequence in comparison to the normal expression level detected in the absence of the siRNA or RNAi agent. Inhibition of expression of a target gene or target sequence by a siRNA or RNAi agent is achieved when the expression level of the target gene mRNA or protein is about 90%, 80%, 70%, 60%, 50%, 40%, 30%, 25%, 20%, 15%, 10%, 5%, or 0% relative to the expression level of the target gene mRNA or protein of a control sample. In another embodiment of the invention, an effective amount is an amount of an siRNA or RNAi agent sufficient to inhibit the proliferation of a mammalian cell overexpressing Myc.

As used herein, the term “isolated” in the context of an isolated nucleic acid molecule is one that is altered or removed from the natural state through human intervention. For example, an RNA naturally present in a living animal is not “isolated.” A synthetic RNA or dsRNA or microRNA molecule partially or completely separated from the coexisting materials of its natural state is “isolated.” Thus, an miRNA molecule that is deliberately delivered to or expressed in a cell is considered an “isolated” nucleic acid molecule.

By “modulate” it is meant that the expression of the gene or level of RNA molecule or equivalent RNA molecules encoding one or more proteins or protein subunits or activity of one or more proteins or protein subunits is up-regulated or down-regulated, such that expression, level, or activity is greater than or less than that observed in the absence of the modulator. For example, the term “modulate” can mean “inhibit,” “up-regulate,” or “down-regulate,” but the use of the word “modulate” is not limited to this definition.

As used herein, “RNA” refers to a molecule comprising at least one ribonucleotide residue. The term “ribonucleotide” means a nucleotide with a hydroxyl group at the 2′-position of a β-D-ribofuranose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of an RNAi agent or internally, for example, at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

As used herein, the term “complementary” refers to nucleic acid sequences that are capable of base-pairing according to the standard Watson-Crick complementary rules. That is, the larger purine will base pair with the smaller pyrimidines to form combinations of guanine paired with cytosine (G:C) and adenine paired with either thymine (A:T) in the case of DNA, or adenine paired with uracil (A:U) in the case of RNA.

As used herein, the term “essentially complementary” with reference to microRNA target sequences refers to microRNA target nucleic acid sequences that are longer than 8 nucleotides that are complementary (an exact match) to at least 8 consecutive nucleotides of the 5′-portion of a microRNA molecule from nucleotide positions 1 to 12, (also referred to as the “seed region”), and are at least 65% complementary (such as at least 70%, at least 75%, at least 80%, at least 85%, at least 90%, at least 95%, or at least 96% identical) across the remainder of the microRNA target nucleic acid sequence as compared to the naturally occurring microRNA.

As used herein, “percent identity” refers to the number of exact matches, expressed as a percentage of the total, between two nucleotide or amino acid sequences. The comparison of sequences and determination of percent identity and similarity between two sequences can be accomplished using a mathematical algorithm of Karlin and Altschul (PNAS 87:2264-2268, 1990), modified as in Karlin and Altschul (PNAS 90:5873-5877, 1993). Such an algorithm is incorporated into the NBLAST and XBLAST programs of Altschul et al. (J. Mol. Biol. 215:403-410, 1990).

As used herein, the term “phenotype” encompasses the meaning known to one of skill in the art, including modulation of the expression of one or more genes as measured by gene expression analysis or protein expression analysis.

As used herein, the term “cancer” means any disease, condition, trait, genotype, or phenotype characterized by unregulated cell growth or replication as is known in the art including cancers of the blood such as leukemias, for example, acute myelogenous leukemia (AML), chronic myelogenous leukemia (CML), acute lymphocytic leukemia (ALL), and chronic lymphocytic leukemia; lymphomas including, but not limited to, Hodgkin and non-Hodgkin lymphomas, Burkitt's lymphoma, and other B-cell lymphomas, myelomas, and myelodysplastic syndrome; AIDS-related cancers such as Kaposi's sarcoma; breast cancers; bone cancers, such as osteosarcoma, chondrosarcomas, Ewing's sarcoma, fibrosarcomas, giant cell tumors, adamantinomas, and chordomas; brain cancers such as meningiomas, glioblastomas, lower-grade astrocytomas, oligodendrocytomas, pituitary tumors, Schwannomas, and metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, colorectal cancer, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease; and any other cancer or proliferative disease, condition, trait, genotype or phenotype that can respond to the modulation of disease related gene expression in a cell or tissue, alone or in combination with other therapies.

As used herein, the term cancer includes the terms “tumor,” “malignant tumor,” and “neoplasm,” as those terms are understood in the art. A cancer cell is a cell derived from a cancer or tumor and includes tumor stem cells.

As used herein, the term to “inhibit the proliferation of a mammalian cell” means to kill the cell or permanently or temporarily arrest the growth of the cell. Inhibition of the proliferation of a mammalian cell can be inferred if the number of such cells, either in an in vitro culture vessel or in a subject, remains constant or decreases after administration of the compositions of the invention. An inhibition of tumor cell proliferation can also be inferred if the absolute number of such cells increases, but the rate of tumor growth decreases. As used herein, “cell death” includes apoptosis and programmed cell death as those terms are understood in the art, and also includes cell cycle arrest. As used herein, the term “reducing the tumor burden in a subject” refers to inhibiting the growth rate of a tumor, slowing or stopping the growth of the tumor, reducing the size of the tumor, or partial or complete remission of the tumor in the subject.

As used herein, the terms “measuring expression levels,” “obtaining an expression level,” and the like includes methods that quantify a gene expression level of, for example, a transcript of a gene, including microRNA (miRNA) or a protein encoded by a gene, as well as methods that determine whether a gene of interest is expressed at all. Thus, an assay that provides a “yes” or “no” result without necessarily providing quantification of an amount of expression is an assay that “measures expression” as that term is used herein. Alternatively, a measured or obtained expression level may be expressed as any quantitative value, for example, a fold-change in expression, up or down, relative to a control gene or relative to the same gene in another sample or a log ratio of expression, or any visual representation thereof, such as, for example, a “heatmap” where a color intensity is representative of the amount of gene expression detected. Exemplary methods for detecting the level of expression of a gene include, but are not limited to, Northern blotting, dot or slot blots, reporter gene matrix (see, for example, U.S. Pat. No. 5,569,588) nuclease protection, RT-PCR, microarray profiling, differential display, Western blot analysis, 2D gel electrophoresis, SELDI-TOF, ICAT, enzyme assay, antibody assay, and the like.

As used herein, an “isolated nucleic acid” is a nucleic acid molecule that exists in a physical form that is non-identical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

The terms “over-expression,” “over-expresses,” “over-expressing” and the like, refer to the state of altering a subject such that expression of one or more genes in said subject is significantly higher, as determined using one or more statistical tests, than the level of expression of said gene or genes in the same unaltered subject or an analogous unaltered subject.

As used herein, a “purified nucleic acid” represents at least 10% of the total nucleic acid present in a sample or preparation. In preferred embodiments, the purified nucleic acid represents at least about 50%, at least about 75%, or at least about 95% of the total nucleic acid in an isolated nucleic acid sample or preparation. Reference to “purified nucleic acid” does not require that the nucleic acid has undergone any purification and may include, for example, chemically synthesized nucleic acid that has not been purified.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least 2-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM (micro Molar).

As used herein, “subject” refers to an organism or to a cell sample, tissue sample, or organ sample derived therefrom including, for example, cultured cell lines, biopsy, blood sample, or fluid sample containing a cell. For example, an organism may be an animal including, but not limited to, an animal such as a cow, a pig, a mouse, a rat, a chicken, a cat, a dog, etc., and is usually a mammal, such as a human. As used herein, a “patient” is a subject who has or may have a disease.

As used herein, the term “treatment” refers to administration of one or more agents or therapeutic compounds for the purpose of alleviating the symptoms of disease, halting or slowing the progression or worsening of those symptoms, or prevention or prophylaxis of disease. For example, successful treatment may include an alleviation of symptoms or halting the progression of cancer, as measured by a reduction in the growth rate of a tumor, a reduction in the size of a tumor, partial or complete remission of the tumor, or increased survival rate or clinical benefit.

Treatment regimens as contemplated herein are well known to those skilled in the art. For example, without limitation, an agent may be administered to a patient in need thereof daily for 7, 14, 21, or 28 days followed by 7 or 14 days without administration of the compound. In some embodiments, the treatment cycle comprises administering the amount of an agent daily for 7 days followed by 7 days without administration of the compound. A treatment cycle may be repeated one or more times to provide a course of treatment. In addition, an agent may be administered once, twice, three times, or four times daily during the administration phase of the treatment cycle. In other embodiments, the methods further comprise administering the amount of an agent once, twice, three times, or four times daily or every other day during a course of treatment.

In some embodiments, the treatment regimens further include administering an agent as part of a treatment cycle. A treatment cycle includes an administration phase during which an agent is given to the subject on a regular basis and a holiday, during which the compound is not administered. For example, the treatment cycle may comprise administering the agent daily for 7, 14, 21, or 28 days, followed by 7 or 14 days without administration of the agent. In some embodiments, the treatment cycle comprises administering the agent daily for 7 days followed by 7 days without administration of the agent. A treatment cycle may be repeated one or more times to provide a course of treatment. In addition, an agent may be administered once, twice, three times, or four times daily during the administration phase of the treatment cycle. In other embodiments, the methods further comprise administering the amount of an agent once, twice, three times, or four times daily or every other day during a course of treatment. A course of treatment refers to a time period during which the subject undergoes treatment for cancer by the present methods. Thus, a course of treatment may extend for one or more treatment cycles or refer to the time period during which the subject receives daily or intermittent doses of an agent.

As used herein, “hypoxia” refers to a reduced oxygen concentration or oxygen tension in tissues, as the term is understood by those of skill in the art. In one embodiment, hypoxia refers to decreased oxygen concentration or tension levels in tumor tissues as compared to normal, non-tumor tissues. In another embodiment, hypoxia refers to oxygen concentrations of 0.2% to 2.0% in tumor tissues or oxygen concentrations of 0.2% to 2.0% at or near the external surface of tumor cells. In some embodiments, hypoxia refers to oxygen concentrations of 1.0% or less in tumor tissues or oxygen concentrations of 1.0% or less at or near the external surface of tumor cells.

As used herein, “hypoxia inducible factor” (HIF) refers to a family of transcription factors that activate transcription of target genes during hypoxia. The HIF family includes the polypeptides HIF-1α (hypoxia-inducible factor 1α), HIF-1β (otherwise known as aryl hydrocarbon receptor nuclear translocator (ARNT)), and HIF-2α(otherwise known as endothelial PAS domain protein 1 (EPAS1)), and naturally occurring isoforms thereof. As used herein, HIF-1α includes isoform 1 (SEQ ID NO:31) and isoform 2 (SEQ ID NO:32). HIF-1β includes isoform 1 (SEQ ID NO:33), isoform 2 (SEQ ID NO:34), and isoform 3 (SEQ ID NO:35). HIF-2α includes SEQ ID NO:36.

As used herein, “metastasis” refers to the process by which cancer cells spread from the primary tumor to other locations in the body.

As used herein, “metastatic potential” refers to the probability or likelihood that tumor cells will spread or metastasize from their current location, for example the primary tumor, to other locations in the body of a subject or patient. The term also refers to the probability that a patient will develop a cancer metastasis.

As use herein, “tumor” refers to an abnormal growth or mass of tissue, or a mass of cancer cells that arise from organs or other solid tissues, and includes the term neoplasm. As used herein, the term tumor generally refers to malignant tumors which are capable of spreading or metastasizing to other parts of the body. As used herein, a “tumor cell” is a cell derived from a tumor or other type of cancer, including non-solid tumors, such as cancers of the blood, including leukemia, multiple myeloma, or lymphoma. The term tumor cell also includes cancer stem cells and tumor stem cells (see for example, Hermann, P. C., et al., “Metastatic Cancer Stem Cells: A New Target for Anti-Cancer Therapy?” Cell Cycle 7:188-193, 2008). As used herein, the terms “tumor” and “cancer” may be used interchangeably.

As used herein, “apoptosis” refers to cell death, including but not limited to “programmed cell death”. In one embodiment, apoptosis refers to death of cancer or tumor cells. As used herein, “synthetic lethal” refers to functional changes in two distinct genes or genetic pathways that result in cell death. In one embodiment, synthetic lethal refers to overexpression of a gene and a microRNA that results in cell death. In another embodiment, synthetic lethal refers to overexpression of one gene and decreased expression or inhibition of another gene, which results in cell death.

As used herein, “polymorphism” refers to a naturally occurring variation in a nucleotide sequence between individuals or between species. For example, a polymorphism may be a single nucleotide polymorphism (SNP), wherein an SNP represents a single nucleotide change between two alleles of a gene. A polymorphism may also encompass more than one nucleotide difference between two related nucleotide sequences or two alleles of a gene.

As used herein, “isoform” refers to one version of a protein or polypeptide that is different from another isoform of the protein or polypeptide between individuals or between species. Different isoforms may be encoded by different genes or from the same gene, for example by alternate splicing of RNA molecules. Isoforms may also arise from single nucleotide polymorphisms (SNPs), wherein an SNP represents a single nucleotide change between two alleles of a gene. Isoforms may be distinguished from other isoforms by size or by amino acid sequence.

### II. ASPECTS AND EMBODIMENTS OF THE INVENTION

One aspect of the invention relates to the use of miR-210 as a biomarker for hypoxia in a cell. Another aspect of the invention relates to the use of miR-210 to predict the likelihood of metastasis of a tumor. Another aspect of the invention relates to the use of miR-210 or miR-210-like siRNA molecules (siRNAs structurally and functionally similar to miR-210) to inhibit the proliferation of tumor cells that overexpress Myc. Another aspect of the invention relates to a method for inhibiting the proliferation of tumor cells expressing Myc by contacting the tumor cells with an inhibitor of an RNA or polypeptide encoded by one or more genes that are down-regulated by miR-210, such as Mnt (SEQ ID NOs:29, 30). In yet another aspect, the invention relates to a method of inhibiting the proliferation of tumor cells that overexpress miR-210 by administering an inhibitor of HIF to the tumor cells.

A. The Use of miR-210 as a Biomarker for Hypoxia in Tumor Cells

In one aspect, the present invention provides a method for determining the presence of hypoxia in tumor cells isolated from a subject. In one embodiment, the method comprises isolating cells from tumors and adjacent non-tumor tissue from a subject; measuring the expression levels of miR-210 in the tumor and non-tumor cells; and comparing the measured expression levels of miR-210 to a hypoxia reference value, wherein expression levels of miR-210 above the hypoxia reference value are indicative of hypoxia in the cells. In another embodiment, the method comprises measuring the amount of miR-210 present in tumor cells from a subject, and comparing the measured amount of miR-210 with a hypoxia reference value, wherein expression levels of mIR-210 greater than the hypoxia reference value are indicative of hypoxia in the cells. As described in Example 1, it has been determined by the inventors that the presence of hypoxia correlates with increased miR-210 expression in tumor cells exposed to hypoxic conditions (1% O₂) relative to tumor cells exposed to normal (ambient) oxygen levels (21% O₂).

In one embodiment of this aspect of the invention, the expression level of miR-210 is determined by measuring the amount the mature microRNA (SEQ ID NO:1). The amount of miR-210 present in cells or tissues can be measured using methods such as nucleic acid hybridization (Lu et al., Nature 435:834-838, 2005), quantitative polymerase chain reaction (Raymond et al., “Simple, Quantitative Primer-Extension PCR Assay for Direct Monitoring of microRNAs and Short-Interfering RNAs,” RNA 11:1737-1744, 2006), incorporated herein by reference, or any other method that is capable of providing a measured level (either as a quantitative amount or as an amount relative to a standard or control amount, i.e., a ratio or a fold-change) of a micro-RNA within a cell or tissue sample. In another embodiment, the expression level of miR-210 is determined by measuring the amount of the primary transcript, pri-miR-210 (SEQ ID NO:2). The amount of pri-miR-210 present in cells or tissues can be measured using methods such as gene expression profiling using microarrays (Jackson et al., “Expression Profiling Reveals Off-Target Gene Regulation by RNAi,” Nat. Biotech. 21:635-637, 2003) or any other method that is capable of providing a measured level (either as a quantitative amount or as an amount relative to a standard or control amount, i.e., a ratio or a fold-change) of an RNA within a cell or tissue sample. In another embodiment, the expression level of miR-210 is determined by measuring the amount of the stem-loop precursor, pre-miR-210 (SEQ ID NO:3).

In one embodiment, the difference between the measured level of miR-210 in the cell sample and the hypoxia reference value is evaluated using one or more statistical tests known in the art. Based upon the outcome of the one or more statistical tests, the measured level of miR-210 is used to classify the tumor as hypoxic or non-hypoxic in a statistically significant fashion. For example, the tumor may be classified as hypoxic if the level of miR-210 in the cell sample is at least 1.5-fold greater than the hypoxia reference value, or at least 2-fold greater than the hypoxia reference value, or at least 3-, 4-, 5-, 6-, 7-, 8-, 9-, 10-, 11-, 12-, 13-, 14-, 15-, 16-, 17-, 18-, 19-, 20-, 25-, or 30-fold greater than hypoxia reference value.

In some embodiments, the hypoxia reference value is determined by measuring the amount of miR-210 in non-tumor cells taken from the subject. The non-tumor cells may be from adjacent, non-involved (normal) tissue from the same tissue-type as the tumor—for example, breast tissue or lung tissue. In other embodiments, the hypoxia reference value is determined by measuring the amount of miR-210 in cells from a plurality of non-tumor samples obtained from one or more subjects. In another embodiment, the hypoxia reference value is determined by measuring the amount of miR-210 in cells that are not exposed to hypoxic conditions. In this embodiment, the cells may be from tumor cell lines or primary cells cultured in vitro. Exemplary methods in accordance with this embodiment are described in Example 1.

B. miR-210 Expression Predicts the Probability of Tumor Metastasis

In another aspect, the present invention provides a method for predicting the likelihood of metastasis of a tumor in a subject based on expression levels of miR-210. This aspect of the invention is useful because it will provide more effective treatment options for patients whose tumors are predicted to metastasize based on expression levels of miR-210. It is contemplated that the likelihood of metastasis is correlated with patient prognosis and disease outcome, whereby a high likelihood of metastasis is correlated with a poor patient prognosis, and a low likelihood of metastasis is correlated with a good patient outcome.

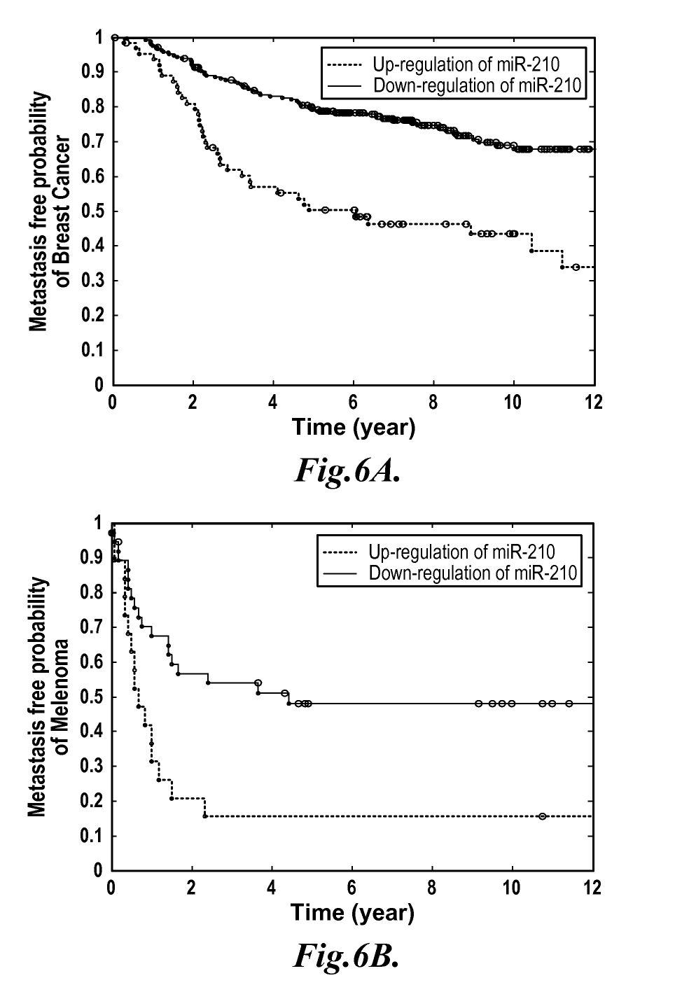

In one embodiment, the measured level of miR-210 in a tumor cell sample is compared to a metastasis reference value, wherein an increase in miR-210 level relative to the metastasis reference value is correlated with the probability of the patient developing metastasis. As described in Example 3, it has been determined that miR-210 expression is upregulated in kidney, lung and breast tumors. As shown in FIG. 6A, upregulation of miR-210 positively correlates with the metastatic potential of breast cancer tumors, and inversely correlates with metastasis-free survival. Thus, in one embodiment, the method is useful for predicting metastasis of kidney, lung and breast tumor cells. As shown in FIG. 6B, upregulation of miR-210 positively correlates with the metastatic potential of melanoma tumors, and inversely correlates with metastasis-free survival. Thus, in another embodiment, the method is useful for predicting metastasis of melanoma cells.

The metastasis reference value may be obtained by measuring miR-210 levels in one or more samples from non-diseased normal tissue from a patient with a tumor. In other embodiments, multiple non-diseased tissue samples can be pooled together and the level of miR-210 in the resulting pool can be used to determine the metastasis reference value. In another embodiment described in Example 3, the metastasis reference value can be obtained by measuring miR-210 or pri-miR-210 levels in cells from a plurality of primary tumors from one or more patients, and taking the average or mean value as the metastasis reference value. Alternatively, the metastasis reference value can be obtained using individual miR-210 measurements from a plurality of the same or different normal tissues from one or more patients using any of a variety of different statistical tests known in the art.

The amount of miR-210 in a cell can be measured as described above. Statistical tests to determine statistically significant changes in the measured level of miR-210 as compared to the metastasis reference value that are predictive of metastasis can be performed as described above.

In one embodiment, measured values statistically higher than the metastasis reference value are correlated with increased probability of developing metastasis, wherein an increased probability of developing metastasis indicates a poor prognosis. As used herein, poor prognosis means a patient is expected to develop a metastasis of the primary tumor within a period of time following diagnosis of the primary tumor or cancer. For example, poor prognosis means the patient is expected to develop a metastasis of the tumor within 1, 2, 3, 4, 5, 6, 8, 10, or 12 years following diagnosis of the primary tumor or cancer.

In other embodiments, measured values statistically lower than the metastasis reference value are correlated with decreased probability of developing metastasis, wherein a decreased probability of developing metastasis indicates a good prognosis. As used herein, good prognosis means a patient is not expected to develop a metastasis of the primary tumor within a period of time following diagnosis of the primary tumor or cancer. For example, good prognosis means the patient is not expected to develop a metastasis of the tumor within 1, 2, 3, 4, 5, 6, 8, 10, or 12 years following diagnosis of the primary tumor or cancer.

In other embodiments, the one or more statistical tests can be used to determine the degree or magnitude of miR-210 expression in tumor cells relative to non-involved (non-tumor) cells from the subject. In one embodiment, a statistically significant change of 1.5- to 2-fold increase in the measured level of miR-210 indicates that the tumor has a low probability of metastasis (i.e., a probability of less than 50%). In another embodiment, a statistically significant change of 2- to 5-fold increase in the measured level of miR-210 indicates that the tumor has a medium probability of metastasis (a probability of approximately 50%). In yet another embodiment, a statistically significant change of 5-fold or greater in the measured level of miR-210 indicates that the tumor has a high probability of metastasis (i.e., a probability of greater than 50%).

In one embodiment, the invention provides a method for predicting the hypoxia response in tumor cells in a subject, wherein the hypoxia response is positively correlated with the probability of metastasis or metastatic potential of the tumor cells. In this embodiment, expression of miR-210 above a hypoxia reference value indicates the tumor cells are hypoxic. Hypoxic tumor cells tend to exhibit increased invasive potential and resistance to conventional therapies (Harris, A. L., Nat. Rev. Cancer 2:38-47, 2002). Therefore, in this embodiment, overexpression of miR-210 serves as a biomarker for both hypoxia and metastatic potential of tumor cells.

C. Introduction of miR-210 into Cells that Overexpress Myc Inhibits Cell Proliferation

In another aspect, the present invention provides methods for inhibiting the proliferation of cells that overexpress the Myc oncogene, for example, cancer cells. In this aspect of the invention, miR-210 siNA is introduced into cells that overexpress Myc to inhibit their proliferation, thereby reducing the tumor burden in the subject. This aspect of the invention is useful in treating cancers that exhibit Myc overexpression.

As used herein, Myc refers to the oncogenes c-Myc, N-Myc, and L-Myc, and variants, polymorphisms, or isoforms thereof. For example, c-Myc includes the nucleotide sequence set forth in SEQ ID NO:23 (Genbank Accession No. NM_{—}002467) and the polypeptide sequence set forth in SEQ ID NO:24 (Genbank Accession No. NP_{—}002458), and any naturally occurring variants, polymorphisms and isoforms thereof. Isoforms of c-Myc include the c-Myc 1 (p67 Myc) and c-Myc2 (p64 Myc) polypeptides with distinct amino-terminal regions resulting from alternate translation initiation codons as described by Nanbru et al., “Alternative Translation of the Proto-Oncogene c-Myc by an Internal Ribosome Entry Site,” J. Biol. Chem. 272:32061-32066, 1997, which is hereby incorporated by reference herein. N-Myc, also known as MYCN, includes the nucleotide sequence set forth in SEQ ID NO:25 (Genbank Accession No. NM_{—}005378) and the polypeptide sequence set forth in SEQ ID NO:26 (Genbank Accession No. NP_{—}005369), and any naturally occurring variants, polymorphisms, and isoforms thereof, including polypeptides with distinct amino-terminal regions, such as those described by Makela et al., “Two N-Myc Polypeptides With Distinct Amino Termini Encoded by the Second and Third Exons of the Gene,” Mol. Cell. Biol. 9:1545-1552, 1989, and Stanton, L. W., and J. M. Bishop, “Alternative Processing of RNA Transcribed From NMYC,” Mol. Cell. Biol. 7:4266-4277, 1987, which are hereby incorporated by reference herein. L-Myc, also known as MYCL1, includes the nucleotide sequence set forth in SEQ ID NO:27 (transcript variant 1) and the polypeptide sequence set forth in SEQ ID NO:28 (isoform 1). L-Myc also includes mRNA transcript variant 1 (Genbank Accession No. NM_{—}001033081, SEQ ID NO:27), variant 2 (Genbank Accession No. NM_{—}001033082), and variant 3 (Genbank Accession No. NM_{—}005376). L-Myc further includes protein isoform 1 (SEQ ID NO:28, encoded by transcript variants 1 and 2) and protein isoform 2 (Genbank Accession No. NP_{—}005367). All Genbank Accession numbers are hereby incorporated by reference herein.

c-Myc is amplified and/or overexpressed in many cancers and tumors including Burkitt's lymphoma, medulloblastoma, hepatocellular carcinoma, lung cancer, breast cancer, colon cancer, pancreatic cancer, ovarian cancer, and prostate cancer (Gardner et al., 2002, Encyclopedia of Cancer, 2d ed.; Wu et al., Am. J. Pathol. 162:1603-1610, 2003; Rao et al., Neoplasia 5:198-205, 2003; Pavelic et al., Anticancer Res. 16:1707-1717. 1996). Overexpression of c-Myc sensitizes cells to stimuli that trigger apoptosis and cell death and can lead to cell-cycle arrest (Nilsson, J. A., and J. L. Cleveland, Oncogene 22:9007-21, 2003). N-Myc is the homolog of c-Myc expressed in neural tissue and is amplified and/or overexpressed in neuroblastomas, medulloblastomas, retinoblastomas, small cell lung carcinoma, glioblastomas, and certain embryonal tumors (Pession and Tonelli, Curr. Cancer Drug Targets 5:273-83, 2005). N-Myc deregulation also occurs in rhabdomyosarcomas (Morgenstern and Anderson, Expert Rev. Anticancer Ther. 6:217-224, 2006). L-Myc is amplified in small cell lung cancer and lung carcinoma cell lines (Nau et al., Nature 318:69-73, 1985) and is amplified and overexpressed in ovarian carcinomas (Wu et al., Am. J. Pathol. 162:1603-1610, 2003).

As used herein, overexpression of Myc in a cell may result from gene amplification, increased transcription of RNA, increased stability or half-life of mRNA, and increased translation of mRNA into protein, or a combination of any of these factors.

In one embodiment, the invention provides a method of inhibiting tumor cell proliferation, comprising measuring the level of Myc protein or nucleic acid in a tumor cell sample; comparing the measured level of Myc with a Myc reference value, and contacting the tumor cells having a level of Myc equal to or greater than the Myc reference value with an amount of an siNA comprising miR-210 effective to inhibit the proliferation of tumor cells.

In another embodiment, the invention provides a method for inhibiting the proliferation of tumor cells, comprising measuring the expression level of Myc in tumor cells, comparing the measured Myc expression level to a Myc reference value; wherein expression levels of Myc greater than the Myc reference value indicate that the cell is sensitized to inhibition of proliferation, and contacting the tumor cells with an amount of miR-210-like siNA effective to inhibit proliferation of the tumor cells. As used herein, the term “sensitized” refers to a state wherein overexpression of Myc results in enhanced susceptibility to other stimuli that prevent tumor cells from dividing and proliferating, thus arresting the cell cycle either temporarily or permanently. For example, as described in Example 7, it has been determined that overexpression of miR-210 triggers apoptosis and cell death in cells that overexpress c-Myc.

In one embodiment, the expression level of at least one of c-Myc RNA (SEQ ID NO:23) or c-Myc protein (SEQ ID NO:24) is measured in tumor cells. In another embodiment, the expression level of at least one of N-Myc RNA (SEQ ID NO:25) or N-Myc protein (SEQ ID NO:26) is measured in tumor cells. In another embodiment, the expression level of at least one of L-Myc RNA (SEQ ID NO:27) or L-Myc protein (SEQ ID NO:28) is measured in tumor cells. These embodiments are understood to include natural variants, polymorphisms, and isoforms of c-Myc, N-Myc and L-Myc, including variants, polymorphisms and isoforms that are expressed in tumor cells.

In one embodiment, the Myc reference value corresponds to the expression level or amount of Myc in normal, non-tumor cells from the same tissue in the subject as the tumor cells. In another embodiment, the Myc reference value corresponds to a mean, median, or average expression level or amount of Myc in a plurality of normal, non-tumor tissue samples from one or more subjects. In yet another embodiment, the Myc reference value corresponds to the level of Myc expressed by human primary tumor cells, tumor stem cells, or tumor cell lines. In this aspect of the invention, the level of Myc protein can be measured by any method known in the art, including Western blot analysis (Benanti et al., “Epigenetic Down-Regulation of ARF Expression Is a Selection Step in Immortalization of Human Fibroblasts by c-Myc,” Mol. Cancer. Res. 5:1181-1189, 2007).

In another embodiment of this aspect of the invention, the method comprises introducing into a tumor cell that overexpresses Myc an effective amount of a miR-210-like siNA, wherein the miR-210-like siNA comprises a guide strand contiguous nucleotide sequence of at least 18 nucleotides, wherein the guide strand comprises a seed region consisting of nucleotide positions 1 to 12, wherein position 1 represents the 5′-end of the guide strand and wherein the seed region comprises a nucleotide sequence of at least 6 contiguous nucleotides that is identical to 6 contiguous nucleotides with SEQ ID NO:4.

In one embodiment exemplified in Example 7, the miR-210-like siNA is a duplex RNA molecule that is introduced into the cell by transfection. As used herein, the term “transfection” includes methods well known in the art for introducing polynucleotides into cells including chemical, lipid, electrical, and viral delivery methods. For example, transfection includes the term transduction as used with viral vectors. In some embodiments, the introduced siNA includes one or more chemically modified nucleotides. An effective amount of siNA is the amount sufficient to cause a measurable inhibition of tumor growth. As used herein, the miR-210-like siNA comprises an miRNA whose seed region sequence contains at least a 6 contiguous nucleotide sequence that is identical to a 6 contiguous nucleotide sequence contained within SEQ ID NO:4.

In another embodiment, the tumor cells overexpressing Myc are contacted with a nucleic acid vector molecule expressing an shRNA gene that comprises miR-210 nucleotide sequence, wherein the shRNA transcription product acts as an RNAi agent. The shRNA gene may encode the mature form of miR-210, such as SEQ ID NO:1, or a miR-210 precursor RNA, such as, for example, SEQ ID NO:2 or SEQ ID NO:3. Alternatively, the shRNA gene may encode any other RNA sequence that is susceptible to processing by endogenous cellular RNA processing enzymes into an active siRNA sequence, wherein the seed region of the active siRNA sequence contains at least a 6 contiguous nucleotide sequence that is identical to a 6 contiguous nucleotide sequence contained within SEQ ID NO:4. An effective amount of shRNA, is the amount sufficient to cause a measurable inhibition of tumor growth.

In some embodiments of this aspect of the invention, the efficacy of an siNA comprising miR-210 for treatment of tumors that overexpress Myc can be determined by measuring tumor volume in a subject using any art-recognized method. For example, caliper measurements may be used to estimate tumor volume using the formula (a×b²)×0.5, where “a” is the largest diameter and “b” is the length perpendicular to the diameter, as described in Example 2. Other useful techniques to detect tumor shrinkage in mammalian subjects such as humans include imaging techniques such as computed tomography (CT) scan and magnetic resonance imaging (MRI) scan. Tumor shrinkage in conjunction with imaging techniques is typically evaluated using the Response Evaluation Criteria In Solid Tumors (RECIST) criteria as described in Jour. Natl. Cancer Instit. 92: 205-216 (2000), incorporated herein by reference. Other techniques may be used to evaluate tumor metabolic activity in vivo including positron emission tomography (PET) and fluorodeoxyglucose (FDG-PET) scans. DNA synthesis may be evaluated using fluorodeoxythymidine (FLT-PET) imaging. For preclinical models, additional techniques may be used that involve the use of tumor cells genetically modified with marker genes such as the luciferase gene.

In one embodiment, patients having tumors that overexpress Myc are treated with a therapeutically sufficient amount of a miR-210 or miR-210-like miRNA, siRNA or shRNA composition. Such treatment may be in combination with one or more DNA damaging agents. Therapeutic miR-210 or miR-210-like compositions comprise a guide strand contiguous nucleotide sequence of at least 18 nucleotides, wherein said guide strand comprises a seed region consisting of nucleotide positions 1 to 12, wherein position 1 represents the 5′-end of said guide strand and wherein said seed region comprises a nucleotide sequence of at least 6 contiguous nucleotides that is identical to 6 contiguous nucleotides within SEQ ID NO:4. In certain embodiments, at least one of the two strands further comprises a 1-4, preferably a 2-nucleotide 3′-overhang. The nucleotide overhang can include any combination of a thymine, uracil, adenine, guanine, or cytosine, or derivatives or analogues thereof. The nucleotide overhang in certain aspects is a 2-nucleotide overhang, where both nucleotides are thymine. Importantly, when the dsRNA comprising the sense and antisense strands is administered, it directs target specific interference and bypasses an interferon response pathway.

In order to enhance the stability of the short interfering nucleic acids, the 3′-overhangs can also be stabilized against degradation. In one embodiment, the 3′-overhangs are stabilized by including purine nucleotides, such as adenosine or guanosine nucleotides. Alternatively, substitution of pyrimidine nucleotides by modified analogues, e.g., substitution of uridine nucleotides in the 3′-overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′-overhang in tissue culture medium.

As used herein, a “3′-overhang” refers to at least one unpaired nucleotide extending from the 3′-end of an siRNA sequence. The 3′-overhang can include ribonucleotides or deoxyribonucleotides or modified ribonucleotides or modified deoxyribonucleotides. The 3′-overhang is preferably from 1 to about 5 nucleotides in length, more preferably from 1 to about 4 nucleotides in length and, most preferably, from about 2 to about 4 nucleotides in length. The 3′-overhang can occur on the sense or antisense sequence, or on both sequences of an RNAi construct. The length of the overhangs can be the same or different for each strand of the duplex. Most preferably, a 3′-overhang is present on both strands of the duplex and the overhang for each strand is 2-nucleotides in length. For example, each strand of the duplex can comprise 3′-overhangs of dithymidylic acid (“tt”) or diuridylic acid (“uu”).

Another aspect of the invention provides chemically modified siNA constructs. For example, the siNA agent can include a non-nucleotide moiety. A chemical modification or other non-nucleotide moiety can stabilize the sense (guide strand) and antisense (passenger strand) sequences against nucleolytic degradation. Additionally, conjugates can be used to increase uptake and target uptake of the siNA agent to particular cell types. Thus, in one embodiment the siNA agent includes a duplex molecule wherein one or more sequences of the duplex molecule are chemically modified. Non-limiting examples of such chemical modifications include phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5′-C-methyl nucleotides, and terminal glyceryl, and/or inverted deoxy abasic residue incorporation. These chemical modifications, when used in siNA agents, can help to preserve RNAi activity of the agents in cells and can increase the serum stability of the siNA agents.

In one embodiment, the first and optionally or preferably the first two internucleotide linkages at the 5′-end of the antisense and/or sense sequences are modified, preferably by a phosphorothioate. In another embodiment, the first, and perhaps the first two, three, or four internucleotide linkages at the 3′-end of a sense and/or antisense sequence are modified, for example, by a phosphorothioate. In another embodiment, the 5-end of both the sense and antisense sequences, and the 3′-end of both the sense and antisense sequences are modified as described.

D. Inhibitors of Mnt Inhibit Cell Proliferation in Cells that Overexpress Myc

In another aspect, the present invention provides methods for inhibiting cell proliferation of cells overexpressing Myc. In this aspect of the invention, a Mnt inhibitor is introduced into cells that overexpress Myc. c-Myc is a transcription factor that forms obligate dimers with Max protein, and the heterodimer then binds to specific DNA sequences to activate transcription of target genes (Grandori et al., Ann. Rev. Cell Dev. Biol. 16:653-699, 2000; Guccione et al., Nat. Cell Biol. 8:764-770, 2006; Rottman and Luscher, Curr. Top. Microbiol. Immunol. 302:63-122, 2006). Mnt interacts with Max to repress transcription and functions as a c-Myc antagonist (Hurlin, P. J., et al., “Deletion of Mnt Leads to Disrupted Cell Cycle Control and Tumorigenesis,” Embo. J. 22:4584-4596, 2003; Walker, W., et al., J. Cell Biol. 169:405-413, 2005).

In one embodiment, the invention provides a method of inhibiting the proliferation of tumor cells in a subject, comprising measuring the expression level of at least one of c-Myc, N-Myc, or L-Myc, or variants, polymorphisms, or isoforms thereof, in tumor cells; comparing the measured expression level of Myc to a Myc reference value, wherein Myc expression levels greater than the Myc reference value indicate that the cell is sensitized to inhibition of proliferation; and contacting the tumor cells with an inhibitor of the expression or activity of Mnt (SEQ ID NO:30), or variants, polymorphisms, or isoforms thereof.

In one embodiment exemplified in Example 7, the Mnt inhibitor is an siNA that inhibits the expression of Mnt. In this embodiment, the anti-Mnt siNA may be duplex siRNA that is introduced into the cells by transfection. Exemplary embodiments of anti-Mnt siRNAs are represented by SEQ ID NOS: 15-17, as shown in Table 1. In some embodiments, the introduced anti-Mnt siNA includes one or more chemically modified nucleotides.

In another embodiment, proliferation of tumor cells overexpressing Myc is inhibited by introduction of a nucleic acid vector molecule expressing an shRNA gene, wherein the shRNA transcription product acts as an RNAi agent that inhibits the expression of Mnt. In one embodiment, the vector expresses an shRNA sequence selected from the group consisting of SEQ ID NOS: 15, 16, and 17, as shown in Table 1.

E. Use of Inhibitors of the Hypoxia Response in Cells that overexpress miR-210

Another aspect of the invention provides a method for treating a disease associated with hypoxia, such as cancer, by inhibiting the hypoxia response in tumor cells. In one embodiment, the method comprises measuring the expression level or amount of miR-210 in tumor cells from a subject; comparing the level or amount of miR-210 present in the tumor cells to a hypoxia reference value, wherein expression levels of miR-210 higher than the hypoxia reference value indicate the tumor cells are hypoxic; and contacting the tumor cells with an inhibitor of one or more genes, RNAs or proteins comprising the hypoxia response pathway, such as HIF-1α, HIF-1β, and HIF-2α, or variants, polymorphisms, or isoforms thereof. Inhibitors of HIFs include camptothecins and related derivatives, such as topotecan, and other small molecule inhibitors. In one embodiment, the inhibitor of the hypoxia response pathway is an siNA that inhibits the expression of one or more hypoxia inducible factors, including HIF-1α, HIF-1β, and HIF-2α. In another embodiment, the inhibitor of the hypoxia response pathway includes inhibitors of the expression or activity of Mnt, and variants, polymorphisms, or isoforms thereof.

F. Use of Inhibitors of miR-210 in Cells that are Hypoxic

Another aspect of the invention provides a method for treating a disease associated with hypoxia, such as cancer, by inhibiting the function of miR-210 in tumor cells. In one embodiment, the method comprises measuring the level or amount of miR-210 in tumor cells from the subject; comparing the measured level or amount of miR-210 present in the tumor cells to a hypoxia reference value, wherein levels of miR-210 higher than the hypoxia reference value indicate the tumor cells are hypoxic; and contacting the tumor cells with a miR-210 inhibitor, thereby inhibiting the proliferation of tumor cells in the subject.

In one embodiment, the inhibitor of miR-210 function comprises an oligonucleotide complementary to at least six contiguous nucleotides of SEQ ID NO:4.

Representative examples of miR-210 inhibitors include anti-microRNAs (anti-miRs), such as those described in Example 4. MicroRNA inhibitors are commercially available, for example, from Exiqon (Denmark), Ambion (Austin, Tex.), and Dharmacon (Lafayette, Colo.), thereby enabling one of skill in the art to practice this method of the invention.

### III. NUCLEIC ACID MOLECULES FOR USE IN THE METHODS OF THE INVENTION

As used herein a “nucleobase” refers to a heterocyclic base such as, for example a naturally occurring nucleobase (i.e., an A, T, G, C, or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, an 8-bromoguanine, an 8-chloroguanine, a bromothymine, an 8-aminoguanine, an 8-hydroxyguanine, an 8-methylguanine, an 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, an 8-bromoadenine, an 8-hydroxyadenine, a 6-hydroxyaminopurine, a 6-thiopurine, a 4-(6-aminohexyl/cytosine), and the like. A nucleobase may be comprised in a nucleoside or nucleotide, using any chemical or natural synthesis method described herein or known to one of ordinary skill in the art. Such nucleobase may be labeled or it may be part of a molecule that is labeled and contains the nucleobase.

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9-position of a purine or a 7-deazapurine to the 1′-position of a 5-carbon sugar. In another non-limiting example, a nucleoside comprising a pyrimidine nucleobase (i.e., C, T, or U) typically covalently attaches a 1-position of a pyrimidine to a 1′-position of a 5-carbon sugar (Kornberg and Baker, DNA Replication, Freeman and Company, New York, 1992).

As used herein, a “nucleotide” refers to a nucleoside further comprising a “backbone moiety.” A backbone moiety generally covalently attaches a nucleotide to another molecule comprising a nucleotide or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. Other types of attachments are known in the art, particularly when a nucleotide comprises derivatives or analogs of a naturally occurring 5-carbon sugar or phosphorus moiety.

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, Nucleotide Analogs Synthesis and Biological Function, Wiley, New York, 1980).

Additional non-limiting examples of nucleosides, nucleotides or nucleic acids comprising 5-carbon sugar and/or backbone moiety derivatives or analogs, include those in: U.S. Pat. No. 5,681,947, which describes oligonucleotides comprising purine derivatives that form triple helixes with and/or prevent expression of dsDNA; U.S. Pat. Nos. 5,652,099 and 5,763,167, which describe nucleic acids incorporating fluorescent analogs of nucleosides found in DNA or RNA, particularly for use as fluorescent nucleic acids probes; U.S. Pat. No. 5,614,617, which describes oligonucleotide analogs with substitutions on pyrimidine rings that possess enhanced nuclease stability; U.S. Pat. Nos. 5,670,663, 5,872,232, and 5,859,221, which describe oligonucleotide analogs with modified 5-carbon sugars (i.e., modified 2′-deoxyfuranosyl moieties) used in nucleic acid detection; U.S. Pat. No. 5,446,137, which describes oligonucleotides comprising at least one 5-carbon sugar moiety substituted at the 4′-position with a substituent other than hydrogen that can be used in hybridization assays; U.S. Pat. No. 5,886,165, which describes oligonucleotides with both deoxyribonucleotides with 3′ to −5′-internucleotide linkages and ribonucleotides with 2′- to 5′-internucleotide linkages; U.S. Pat. No. 5,714,606, which describes a modified internucleotide linkage wherein a 3′-position oxygen of the internucleotide linkage is replaced by a carbon to enhance the nuclease resistance of nucleic acids; U.S. Pat. No. 5,672,697, which describes oligonucleotides containing one or more 5′-methylene phosphonate internucleotide linkages that enhance nuclease resistance; U.S. Pat. Nos. 5,466,786 and 5,792,847, which describe the linkage of a substituent moiety that may comprise a drug or label to the 2′-carbon of an oligonucleotide to provide enhanced nuclease stability and ability to deliver drugs or detection moieties; U.S. Pat. No. 5,223,618, which describes oligonucleotide analogs with a 2- or 3-carbon backbone linkage attaching the 4′-position and 3′-position of adjacent 5-carbon sugar moiety to enhanced cellular uptake, resistance to nucleases and hybridization to target RNA; U.S. Pat. No. 5,470,967, which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289, and 5,602,240, which describe oligonucleotides with three or four atom linker moiety replacing phosphodiester backbone moiety used for improved nuclease resistance, cellular uptake, and regulating RNA expression; U.S. Pat. No. 5,858,988, which describes hydrophobic carrier agent attached to the 2′-O position of oligonucleotides to enhance their membrane permeability and stability; U.S. Pat. No. 5,214,136, which describes oligonucleotides conjugated to anthraquinone at the 5′-terminus that possess enhanced hybridization to DNA or RNA; enhanced stability to nucleases; U.S. Pat. No. 5,700,922, which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154, which describes RNA linked to a DNA to form a DNA-RNA hybrid; U.S. Pat. No. 5,728,525, which describes the labeling of nucleoside analogs with a universal fluorescent label.

Additional teachings for nucleoside analogs and nucleic acid analogs are U.S. Pat. No. 5,728,525, which describes nucleoside analogs that are end-labeled; U.S. Pat. Nos. 5,637,683, 6,251,666 (L-nucleotide substitutions), and 5,480,980 (7-deaza-2′ deoxyguanosine nucleotides and nucleic acid analogs thereof).

shRNA Mediated Suppression

Alternatively, certain of the nucleic acid molecules of the instant invention can be expressed within cells from eukaryotic promoters (e.g., Izant and Weintraub, Science 229:345, 1985; McGarry and Lindquist, PNAS USA 83:399, 1986; Scanlon et al., PNAS USA 88:10591-95, 1991; Kashani-Sabet et al., Antisense Res. Dev. 2:3-15, 1992; Dropulic et al., J. Virol. 66:1432-41, 1992; Weerasinghe et al., J. Virol. 65:5531-4, 1991; Ojwang et al., PNAS USA 89:10802-06, 1992; Chen et al., Nucleic Acids Res. 20:4581-89, 1992; Sarver et al., Science 247:1222-25, 1990; Thompson et al., Nucleic Acids Res. 23:2259, 1995; Good et al., Gene Therapy 4:45, 1997). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., International PCT Publication No. WO 93/23569, and Sullivan et al., International PCT Publication No. WO 94/02595; Ohkawa et al., Nucleic Acids Symp. Ser. 27:15-6, 1992; Taira et al., Nucleic Acids Res. 19:5125-30, 1991; Ventura et al., Nucleic Acids Res. 21:3249-55, 1993; Chowrira et al., J. Biol. Chem. 269:25856, 1994). Gene therapy approaches specific to the CNS are described by Blesch et al., Drug News Perspect. 13:269-280, 2000; Peterson et al., Cent. Nerv. Syst. Dis. 485:508, 2000; Peel and Klein, J. Neurosci. Methods 98:95-104, 2000; Hagihara et al., Gene Ther. 7:759-763, 2000; and Herrlinger et al., Methods Mol. Med. 35:287-312, 2000. AAV-mediated delivery of nucleic acid to cells of the nervous system is further described by Kaplitt et al., U.S. Pat. No. 6,180,613.

In another aspect of the invention, RNA molecules of the present invention are preferably expressed from transcription units (see, for example, Couture et al., TIG. 12:510, 1996) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient or subject followed by reintroduction into the patient or subject, or by any other means that would allow for introduction into the desired target cell (for a review see Couture et al., TIG. 12:510, 1996).

In one aspect the invention features an expression vector comprising a nucleic acid sequence encoding at least one of the nucleic acid molecules of the instant invention is disclosed. The nucleic acid sequence encoding the nucleic acid molecule of the instant invention is operably linked in a manner which allows expression of that nucleic acid molecule.

In another aspect the invention features an expression vector comprising (a) a transcription initiation region (e.g., eukaryotic pol I, II, or III initiation region); (b) a transcription termination region (e.g., eukaryotic pol I, II, or III termination region); and (c) a nucleic acid sequence encoding at least one of the nucleic acid molecules of the instant invention; and wherein said sequence is operably linked to said initiation region and said termination region in a manner that allows expression and/or delivery of said nucleic acid molecule. The vector can optionally include an open reading frame (ORF) for a protein operably linked on the 5′-side or the 3′-side of the sequence encoding the nucleic acid molecule of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, PNAS USA 87:6743-7, 1990; Gao and Huang, Nucleic Acids Res. 21:2867-72, 1993; Lieber et al., Methods Enzymol., 217:47-66, 1993; Zhou et al., Mol. Cell Biol. 10:4529-37, 1990).

Several investigators have demonstrated that nucleic acid molecules encoding shRNAs or microRNAs expressed from such promoters can function in mammalian cells (Brummelkamp et al., Science 296:550-553, 2002; Paddison et al., Nat. Methods 1:163-67, 2004; McIntyre and Fanning, BMC Biotechnology 6:1, January 2006; Taxman et al., BMC Biotechnology 6:7, January 2006). The above shRNA or microRNA transcription units can be incorporated into a variety of vectors for introduction into mammalian cells including, but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising nucleic acid sequence encoding at least one of the nucleic acid molecules of the invention, in a manner which allows expression of that nucleic acid molecule. The expression vector comprises in one embodiment: (a) a transcription initiation region; (b) a transcription termination region; (c) a nucleic acid sequence encoding at least one said nucleic acid molecule; and wherein said sequence is operably linked to said initiation region and said termination region, in a manner that allows expression and/or delivery of said nucleic acid molecule.

In another embodiment, the expression vector comprises (a) a transcription initiation region; (b) a transcription termination region; (c) an open reading frame; and (d) a nucleic acid sequence encoding at least one said nucleic acid molecule, wherein said sequence is operably linked to the 3′-end of said open reading frame; and wherein said sequence is operably linked to said initiation region, said open reading frame and said termination region in a manner that allows expression and/or delivery of said nucleic acid molecule. In yet another embodiment the expression vector comprises (a) a transcription initiation region; (b) a transcription termination region; (c) an intron; and (d) a nucleic acid sequence encoding at least one said nucleic acid molecule; and wherein said sequence is operably linked to said initiation region, said intron and said termination region, in a manner that allows expression and/or delivery of said nucleic acid molecule.

In another embodiment, the expression vector comprises (a) a transcription initiation region; (b) a transcription termination region; (c) an intron; (d) an open reading frame; and (e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region in a manner that allows expression and/or delivery of said nucleic acid molecule.

### IV. MODIFIED SINA MOLECULES

Any of the siNA constructs described herein can be modified and evaluated for use in the methods of the invention as described below.

An siNA construct may be susceptible to cleavage by an endonuclease or exonuclease, such as, for example, when the siNA construct is introduced into the body of a subject. Methods can be used to determine sites of cleavage, e.g., endo- and exonucleolytic cleavage on an RNAi construct and to determine the mechanism of cleavage. An siNA construct can be modified to inhibit such cleavage.

Exemplary modifications include modifications that inhibit endonucleolytic degradation, including the modifications described herein. Particularly favored modifications include 2′-modification, e.g., a 2′-O-methylated nucleotide, or 2′-deoxy nucleotide (e.g., 2′ deoxy-cytodine), or a 2′-fluoro, difluorotoluoyl, 5-Me-2′-pyrimidines, 5-allyl-amino-pyrimidines, 2′-O-methoxyethyl, 2′-hydroxy, or 2′-ara-fluoro nucleotide, or a locked nucleic acid (LNA), extended nucleic acid (ENA), hexose nucleic acid (HNA), or cyclohexene nucleic acid (CeNA). In one embodiment, the 2′-modification is on the uridine of at least one 5′-uridine-adenine-3′ (5′-UA-3′) dinucleotide, at least one 5′-uridine-guanine-3′ (5′-UG-3′) dinucleotide, at least one 5′-uridine-uridine-3′ (5′-UU-3′) dinucleotide, or at least one 5′-uridine-cytidine-3′ (5′-UC-3′) dinucleotide, or on the cytidine of at least one 5′-cytidine-adenine-3′ (5′-CA-3′) dinucleotide, at least one 5′-cytidine-cytidine-3′ (5′-CC-3′) dinucleotide, or at least one 5′-cytidine-uridine-3′ (5′-CU-3′) dinucleotide. The 2′-modification can also be applied to all the pyrimidines in an siNA construct. In one preferred embodiment, the 2′-modification is a 2′OMe modification on the sense strand of an siNA construct. In a more preferred embodiment the 2′-modification is a 2′-fluoro modification, and the 2′-fluoro is on the sense (passenger) or antisense (guide) strand or on both strands.

Modification of the backbone, e.g., with the replacement of an O with an S in the phosphate backbone, e.g., the provision of a phosphorothioate modification can be used to inhibit endonuclease activity. In some embodiments, an siNA construct has been modified by replacing one or more ribonucleotides with deoxyribonucleotides. Preferably, adjacent deoxyribonucleotides are joined by phosphorothioate linkages, and the siNA construct does not include more than four consecutive deoxyribonucleotides on the sense or the antisense strands. Replacement of the U with a C5 amino linker; replacement of an A with a G (sequence changes are preferred to be located on the sense strand and not the antisense strand); or modification of the sugar at the 2′, 6′, 7′, or 8′ position can also inhibit endonuclease cleavage of the siNA construct. Preferred embodiments are those in which one or more of these modifications are present on the sense but not the antisense strand, or embodiments where the antisense strand has fewer of such modifications.

Exemplary modifications also include those that inhibit degradation by exonucleases. In one embodiment, an siNA construct includes a phosphorothioate linkage or P-alkyl modification in the linkages between one or more of the terminal nucleotides of an siNA construct. In another embodiment, one or more terminal nucleotides of an siNA construct include a sugar modification, e.g., a 2′- or 3′-sugar modification. Exemplary sugar modifications include, for example, a 2′-O-methylated nucleotide, 2′-deoxy nucleotide (e.g., deoxy-cytodine), 2′-deoxy-2′-fluoro (2′-F) nucleotide, 2′-O-methoxyethyl (2′-O-MOE), 2′-O-aminopropyl (2′-O-AP), 2′-O—N-methylacetamido (2′-O-NMA), 2′-O-dimethylaminoethlyoxyethyl (2′-DMAEOE), 2′-O-dimethylaminoethyl (2′-O-DMAOE), 2′-O-dimethylaminopropyl (2′-O-AP), 2′-hydroxy nucleotide, or a 2′-ara-fluoro nucleotide, or a locked nucleic acid (LNA), extended nucleic acid (ENA), hexose nucleic acid (HNA), or cyclohexene nucleic acid (CeNA). A 2′-modification is preferably 2′OMe, more preferably, 2′-fluoro.

The modifications described to inhibit exonucleolytic cleavage can be combined onto a single siNA construct. For example, in one embodiment, at least one terminal nucleotide of an siNA construct has a phosphorothioate linkage and a 2′-sugar modification, e.g., a 2′F or 2′OMe modification. In another embodiment, at least one terminal nucleotide of an siNA construct has a 5′ Me-pyrimidine and a 2′ sugar modification, e.g., a 2′F or 2′OMe modification.

To inhibit exonuclease cleavage, an siNA construct can include a nucleobase modification, such as a cationic modification, such as a 3′-abasic cationic modification. The cationic modification can be, e.g., an alkylamino-dT (e.g., a C6 amino-dT), an allylamino conjugate, a pyrrolidine conjugate, a pthalamido or a hydroxyprolinol conjugate, on one or more of the terminal nucleotides of the siNA construct. In one embodiment, an alkylamino-dT conjugate is attached to the 3′ end of the sense or antisense strand of an RNAi construct. In another embodiment, a pyrrolidine linker is attached to the 3′- or 5′-end of the sense strand or the 3′-end of the antisense strand. In one embodiment, an allyl amine uridine is on the 3′- or 5′-end of the sense strand and not on the 5′-end of the antisense strand.

In one embodiment, the siNA construct includes a conjugate on one or more of the terminal nucleotides of the siNA construct. The conjugate can be, for example, a lipophile, a terpene, a protein binding agent, a vitamin, a carbohydrate, a retiniod, or a peptide. For example, the conjugate can be naproxen, nitroindole (or another conjugate that contributes to stacking interactions), folate, ibuprofen, cholesterol, retinoids, PEG, or a C5-pyrimidine linker. In other embodiments, the conjugates are glyceride lipid conjugates (e.g., a dialkyl glyceride derivatives), vitamin E conjugates, or thio-cholesterols. In one embodiment, conjugates are on the 3′-end of the antisense strand or on the 5′- or 3′-end of the sense strand and the conjugates are not on the 3′-end of the antisense strand and on the 3′-end of the sense strand.

In one embodiment, the conjugate is naproxen, and the conjugate is on the 5′- or 3′-end of the sense or antisense strands. In one embodiment, the conjugate is cholesterol and the conjugate is on the 5′- or 3′-end of the sense strand and not present on the antisense strand. In some embodiments, the cholesterol is conjugated to the siNA construct by a pyrrolidine linker, or serinol linker, aminooxy, or hydroxyprolinol linker. In other embodiments, the conjugate is a dU-cholesterol, or cholesterol is conjugated to the siNA construct by a disulfide linkage. In another embodiment, the conjugate is cholanic acid, and the cholanic acid is attached to the 5′- or 3′-end of the sense strand or the 3′-end of the antisense strand. In one embodiment, the cholanic acid is attached to the 3′-end of the sense strand and the 3′-end of the antisense strand. In another embodiment, the conjugate is PEGS, PEG20, naproxen, or retinal.

In another embodiment, one or more terminal nucleotides have a 2′- to 5′-linkage. In certain embodiments, a 2′- to 5′-linkage occurs on the sense strand, e.g., the 5′-end of the sense strand.

In one embodiment, an siNA construct includes an L-sugar, preferably at the 5′- or 3′-end of the sense strand.

In one embodiment, an siNA construct includes a methylphosphonate at one or more terminal nucleotides to enhance exonuclease resistance, e.g., at the 3′-end of the sense or antisense strands of the construct.

In one embodiment, an siRNA construct has been modified by replacing one or more ribonucleotides with deoxyribonucleotides. In another embodiment, adjacent deoxyribonucleotides are joined by phosphorothioate linkages. In one embodiment, the siNA construct does not include more than four consecutive deoxyribonucleotides on the sense or the antisense strands. In another embodiment, all of the ribonucleotides have been replaced with modified nucleotides that are not ribonucleotides.

In some embodiments, an siNA construct having increased stability in cells and biological samples includes a difluorotoluoyl (DFT) modification, e.g., 2,4-difluorotoluoyl uracil, or a guanidine to inosine substitution.

The methods can be used to evaluate a candidate siNA, e.g., a candidate siRNA construct, which is unmodified or which includes a modification, e.g., a modification that inhibits degradation, targets the dsRNA molecule, or modulates hybridization. Such modifications are described herein. A cleavage assay can be combined with an assay to determine the ability of a modified or non-modified candidate to silence the target transcript. For example, one might (optionally) test a candidate to evaluate its ability to silence a target (or off-target sequence), evaluate its susceptibility to cleavage, modify it (e.g., as described herein, e.g., to inhibit degradation) to produce a modified candidate, and test the modified candidate for one or both of the ability to silence and the ability to resist degradation. The procedure can be repeated. Modifications can be introduced one at a time or in groups. It will often be convenient to use a cell-based method to monitor the ability to silence a target RNA. This can be followed by a different method, e.g., a whole animal method, to confirm activity.

Chemically synthesizing nucleic acid molecules with modifications (base, sugar, and/or phosphate) can prevent their degradation by serum ribonucleases, which can increase their potency (see, e.g., Eckstein et al., International Publication No. WO 92/07065; Perrault et al., Nature 344:565, 1990; Pieken et al., Science 253:314, 1991; Usman and Cedergren, Trends in Biochem. Sci. 17:334, 1992; Burgin et al., Biochemistry 35:14090, 1996; Usman et al., International PCT Publication No. WO 93/15187; and Rossi et al., International PCT Publication No. WO 91/03162; Sproat, U.S. Pat. No. 5,334,711; Gold et al., U.S. Pat. No. 6,300,074; and Vargeese et al., U.S. Patent Publication No. 2006/021733). All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

Chemically modified siNA molecules for use in modulating or attenuating expression of two or more genes down-regulated by miR-210-like miRNAs are also within the scope of the invention. Described herein are isolated siNA agents, e.g., RNA molecules (chemically modified or not, double-stranded, or single-stranded) that mediate RNAi to inhibit expression of two or more genes that are down-regulated by miR-210-like siNAs.

The siNA agents discussed herein include otherwise unmodified RNA as well as RNAs that have been chemically modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al., Nucleic Acids Res. 22:2183-2196, 1994. Such rare or unusual RNAs, often termed modified RNAs (apparently because they are typically the result of a post-transcriptional modification) are within the term unmodified RNA, as used herein.

Modified RNA as used herein refers to a molecule in which one or more of the components of the nucleic acid, namely sugars, bases, and phosphate moieties that are the components of the RNAi duplex, are different from that which occur in nature, preferably different from that which occurs in the human body. While they are referred to as modified “RNAs,” they will of course, because of the modification, include molecules that are not RNAs. Nucleoside surrogates are molecules in which the ribophosphate backbone is replaced with a non-ribophosphate construct that allows the bases to the presented in the correct spatial relationship such that hybridization is substantially similar to what is seen with a ribophosphate backbone, e.g., non-charged mimics of the ribophosphate backbone. Examples of all of the above are discussed herein.

Modifications described herein can be incorporated into any double-stranded RNA and RNA-like molecule described herein, e.g., an siNA construct. It may be desirable to modify one or both of the antisense and sense strands of an siNA construct. As nucleic acids are polymers of subunits or monomers, many of the modifications described below occur at a position which is repeated within a nucleic acid, e.g., a modification of a base, or a phosphate moiety, or the non-linking O of a phosphate moiety. In some cases the modification will occur at all of the subject positions in the nucleic acid but in many, and in fact in most, cases it will not.

By way of example, a modification may occur at a 3′- or 5′-terminal position, may occur in a terminal region, e.g., at a position on a terminal nucleotide or in the last 2, 3, 4, 5, or 10 nucleotides of a strand. A modification may occur in a double strand region, a single strand region, or in both. For example, a phosphorothioate modification at a non-linking O position may only occur at one or both termini, may only occur in a terminal regions, e.g., at a position on a terminal nucleotide or in the last 2, 3, 4, 5, or nucleotides of a strand, or may occur in double strand and single strand regions, particularly at termini. Similarly, a modification may occur on the sense strand, antisense strand, or both. In some cases, a modification may occur on an internal residue to the exclusion of adjacent residues. In some cases, the sense and antisense strand will have the same modifications or the same class of modifications, but in other cases the sense and antisense strand will have different modifications, e.g., in some cases it may be desirable to modify only one strand, e.g., the sense strand. In some cases, the sense strand may be modified, e.g., capped in order to promote insertion of the anti-sense strand into the RISC complex.

Other suitable modifications that can be made to a sugar, base, or backbone of an siNA construct are described in U.S. Patent Publication Nos. US 2006/0217331 and US2005/0020521, International PCT Publication Nos. WO2003/70918 and WO2005/019453, and International PCT Patent Application No. PCT/US2004/01193. An siNA construct can include a non-naturally occurring base, such as the bases described in any one of the above mentioned references. See also International Patent Application No. PCT/US2004/011822. An siNA construct can also include a non-naturally occurring sugar, such as a non-carbohydrate cyclic carrier molecule. Exemplary features of non-naturally occurring sugars for use in siNA agents are described in International PCT Patent Application No. PCT/US2004/11829.

Two prime objectives for the introduction of modifications into siNA constructs of the invention are their stabilization towards degradation in biological environments and the improvement of pharmacological properties, e.g., pharmacodynamic properties. There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications (for a review see Usman and Cedergren, TIBS 17:334-339, 1992; Usman et al., Nucleic Acids Symp. Ser. 31:163, 1994; Burgin et al., Biochemistry 35:14090, 1996). Sugar modification of nucleic acid molecules has been extensively described in the art (see Eckstein et al., International PCT Publication No. WO 92/07065; Perrault et al., Nature 344:565-568, 1990; Pieken et al., Science 253:314-317, 1991; Usman and Cedergren, Trends in Biochem. Sci. 17:334-339, 1992; Usman et al., International Patent Publication No. WO 93/15187; Sproat, U.S. Pat. No. 5,334,711; and Beigelman et al., J. Biol. Chem. 270:25702, 1995; Beigelman et al., International Patent Publication No. WO 97/26270; Beigelman et al., U.S. Pat. No. 5,716,824; Usman et al., U.S. Pat. No. 5,627,053; Woolf et al., International Patent Publication No. WO 98/13526; Thompson et al., U.S. Patent Application No. 60/082,404, which was filed on Apr. 20, 1998; Karpeisky et al., Tetrahedron Lett. 39:1131, 1998; Earnshaw and Gait, Biopolymers (Nucleic Acid Sciences) 48:39-55, 1998; Verma and Eckstein, Annu. Rev. Biochem. 67:99-134, 1998; and Burlina et al., Bioorg. Med. Chem. 5:1999-2010, 1997). Such publications describe general methods and strategies to determine the location of incorporation of sugar, base, and/or phosphate modifications and the like into nucleic acid molecules without modulating catalysis. In view of such teachings, similar modifications can be used as described herein to modify the siNA molecules of the instant invention so long as the ability of siNA to promote RNAi in cells is not significantly inhibited.

Modifications may be modifications of the sugar-phosphate backbone. Modifications may also be modification of the nucleoside portion. Optionally, the sense strand is a RNA or RNA strand comprising 5%, 10%, 20%, 30%, 40%, 50%, 60%, 70%, 80%, 90% or 100% modified nucleotides. In one embodiment, the sense polynucleotide is an RNA strand comprising a plurality of modified ribonucleotides. Likewise, in other embodiments, the RNA antisense strand comprises one or more modifications. For example, the RNA antisense strand may comprise no more than 5%, 10%, 20%, 30%, 40%, 50% or 75% modified nucleotides. The one or more modifications may be selected so as increase the hydrophobicity of the double-stranded nucleic acid, in physiological conditions, relative to an unmodified double-stranded nucleic acid having the same designated sequence.

In certain embodiments, the siNA construct comprising the one or more modifications has a logP value at least 0.5 logP unit less than the logP value of an otherwise identical unmodified siRNA construct. In another embodiment, the siNA construct comprising the one or more modifications has at least 1, 2, 3, or even 4 logP units less than the logP value of an otherwise identical unmodified siRNA construct. The one or more modifications may be selected so as increase the positive charge (or increase the negative charge) of the double-stranded nucleic acid, in physiological conditions, relative to an unmodified double-stranded nucleic acid having the same designated sequence. In certain embodiments, the siNA construct comprising the one or more modifications has an isoelectric pH (pI) that is at least 0.25 units higher than the otherwise identical unmodified siRNA construct. In another embodiment, the sense polynucleotide comprises a modification to the phosphate-sugar backbone selected from the group consisting of: a phosphorothioate moiety, a phosphoramidate moiety, a phosphodithioate moiety, a PNA moiety, an LNA moiety, a 2′-O-methyl moiety and a 2′-deoxy-2′ fluoride moiety.

In certain embodiments, the RNAi construct is a hairpin nucleic acid that is processed to an siRNA inside a cell. Optionally, each strand of the double-stranded nucleic acid may be 19 to 100 base pairs long, and preferably 19 to 50 or 19 to 30 base pairs long.

An siNAi construct can include an internucleotide linkage (e.g., the chiral phosphorothioate linkage) useful for increasing nuclease resistance. In addition, or in the alternative, an siNA construct can include a ribose mimic for increased nuclease resistance. Exemplary internucleotide linkages and ribose mimics for increased nuclease resistance are described in International Patent Application No. PCT/US2004/07070.

An siRNAi construct can also include ligand-conjugated monomer subunits and monomers for oligonucleotide synthesis. Exemplary monomers are described, for example, in U.S. patent application Ser. No. 10/916,185.

An siNA construct can have a ZXY structure, such as is described in co-owned International PCT Patent Application No. PCT/US2004/07070. Likewise, an siNA construct can be complexed with an amphipathic moiety. Exemplary amphipathic moieties for use with siNA agents are described in International PCT Patent Application No. PCT/US2004/07070.

The sense and antisense sequences of an siNAi construct can be palindromic. Exemplary features of palindromic siNA agents are described in International Patent PCT Application No. PCT/US2004/07070.

In another embodiment, the siNA construct of the invention can be complexed to a delivery agent that features a modular complex. The complex can include a carrier agent linked to one or more of (preferably two or more, more preferably all three of): (a) a condensing agent (e.g., an agent capable of attracting, e.g., binding, a nucleic acid, e.g., through ionic or electrostatic interactions); (b) a fusogenic agent (e.g., an agent capable of fusing and/or being transported through a cell membrane); and (c) a targeting group, e.g., a cell or tissue targeting agent, e.g., a lectin, glycoprotein, lipid or protein, e.g., an antibody, that binds to a specified cell type. iRNA agents complexed to a delivery agent are described in International PCT Patent Application No. PCT/US2004/07070.

The siNA construct of the invention can have non-canonical pairings, such as between the sense and antisense sequences of the iRNA duplex. Exemplary features of non-canonical iRNA agents are described in International Patent Application No. PCT/US2004/07070.

In one embodiment, nucleic acid molecules of the invention include one or more (e.g., about 1, 2, 3, 4, 5, 6, 7, 8, 9, 10, or more) G-clamp nucleotides. A G-clamp nucleotide is a modified cytosine analog, wherein the modifications confer the ability to hydrogen bond both Watson-Crick and Hoogsteen faces of a complementary guanine within a duplex, see, for example, Lin and Matteucci, J. Am. Chem. Soc. 120:8531-8532, 1998. A single G-clamp analog substitution within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets, complementary sequences, or template strands. In another embodiment, nucleic acid molecules of the invention include one or more (e.g., about 1, 2, 3, 4, 5, 6, 7, 8, 9, 10, or more) LNA “locked nucleic acid” nucleotides such as a 2′,4′-C methylene bicyclo nucleotide (see for example Wengel et al., International PCT Patent Publication Nos. WO 00/66604 and WO 99/14226).

An siNA agent of the invention, can be modified to exhibit enhanced resistance to nucleases. An exemplary method proposes identifying cleavage sites and modifying such sites to inhibit cleavage. An exemplary dinucleotides 5′-UA-3′,5′-UG-3′,5′-CA-3′, 5′-UU-3′, or 5′-CC-3′ as disclosed in PCT/US2005/018931 may serve as a cleavage site.

For increased nuclease resistance and/or binding affinity to the target, a siRNA agent, e.g., the sense and/or antisense strands of the iRNA agent, can include, for example, 2′-modified ribose units and/or phosphorothioate linkages, e.g., the 2′-hydroxyl group (OH) can be modified or replaced with a number of different “oxy” or “deoxy” substituents.

Examples of “oxy”-2′ hydroxyl group modifications include alkoxy or aryloxy (or, e.g., R.dbd.H, alkyl, cycloalkyl, aryl, aralkyl, heteroaryl, or sugar); polyethyleneglycols (PEG), O(CH₂CH₂O)ₙCH₂CH₂OR; “locked” nucleic acids (LNA) in which the 2′-hydroxyl is connected, e.g., by a methylene bridge, to the 4′-carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino), and aminoalkoxy, O(CH₂)ₙAMINE (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e., deoxyribose sugars, which are of particular relevance to the overhang portions of partially dsRNA); halo (e.g., fluoro); amino (e.g., NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with, e.g., an amino functionality. In one embodiment, the substitutents are 2′-methoxyethyl, 2′-OCH₃, 2′-O-allyl, 2′-C-allyl, and 2′-fluoro.

In another embodiment, to maximize nuclease resistance, the 2′-modifications may be used in combination with one or more phosphate linker modifications (e.g., phosphorothioate). The so-called “chimeric” oligonucleotides are those that contain two or more different modifications.

In certain embodiments, all the pyrimidines of a siNA agent carry a 2′-modification, and the molecule therefore has enhanced resistance to endonucleases. Enhanced nuclease resistance can also be achieved by modifying the 5′-nucleotide resulting, for example, in at least one 5′-uridine-adenine-3′ (5′-UA-3′) dinucleotide wherein the uridine is a 2′-modified nucleotide; at least one 5′-uridine-guanine-3′ (5′-UG-3′) dinucleotide, wherein the 5′-uridine is a 2′-modified nucleotide; at least one 5′-cytidine-adenine-3′ (5′-CA-3′) dinucleotide, wherein the 5′-cytidine is a 2′-modified nucleotide; at least one 5′-uridine-uridine-3′ (5′-UU-3′) dinucleotide, wherein the 5′-uridine is a 2′-modified nucleotide; or at least one 5′-cytidine-cytidine-3′ (5′-CC-3′) dinucleotide, wherein the 5′-cytidine is a 2′-modified nucleotide. The siNA agent can include at least 2, at least 3, at least 4 or at least 5 of such dinucleotides. In some embodiments, the 5′-most pyrimidines in all occurrences of the sequence motifs 5′-UA-3′, 5′-CA-3′,5′-UU-3′, and 5′-UG-3′ are 2′-modified nucleotides. In other embodiments, all pyrimidines in the sense strand are 2′-modified nucleotides, and the 5′-most pyrimidines in all occurrences of the sequence motifs 5′-UA-3′ and 5′-CA-3′. In one embodiment, all pyrimidines in the sense strand are 2′-modified nucleotides, and the 5′-most pyrimidines in all occurrences of the sequence motifs 5′-UA-3′,5′-CA-3′,5′-UU-3′, and 5′-UG-3′ are 2′-modified nucleotides in the antisense strand. The latter patterns of modifications have been shown to maximize the contribution of the nucleotide modifications to the stabilization of the overall molecule towards nuclease degradation, while minimizing the overall number of modifications required to a desired stability, see International PCT Application No. PCT/US2005/018931. Additional modifications to enhance resistance to nucleases may be found in U.S. Patent Publication No. US 2005/0020521, and International PCT Patent Publication Nos. WO2003/70918 and WO2005/019453.

The inclusion of furanose sugars in the oligonucleotide backbone can also decrease endonucleolytic cleavage. Thus, in one embodiment, the siNA of the invention can be modified by including a 3′-cationic group, or by inverting the nucleoside at the 3′-terminus with a 3′-3′-linkage. In another alternative, the 3′-terminus can be blocked with an aminoalkyl group, e.g., a 3′ C5-aminoalkyl dT. Other 3′ conjugates can inhibit 3′-5′-exonucleolytic cleavage. While not being bound by theory, a 3′-conjugate, such as naproxen or ibuprofen, may inhibit exonucleolytic cleavage by sterically blocking the exonuclease from binding to the 3′-end of oligonucleotide. Even small alkyl chains, aryl groups, or heterocyclic conjugates or modified sugars (D-ribose, deoxyribose, glucose, etc.) can block 3′-5′-exonucleases.

Similarly, 5′-conjugates can inhibit 5′-3′ exonucleolytic cleavage. While not being bound by theory, a 5′-conjugate, such as naproxen or ibuprofen, may inhibit exonucleolytic cleavage by sterically blocking the exonuclease from binding to the 5′-end of oligonucleotide. Even small alkyl chains, aryl groups, or heterocyclic conjugates or modified sugars (D-ribose, deoxyribose, glucose etc.) can block 3′-5′-exonucleases.

An alternative approach to increasing resistance to a nuclease by an siNA molecule proposes including an overhang to at least one or both strands of an duplex siNA. In some embodiments, the nucleotide overhang includes 1 to 4, preferably 2 to 3, unpaired nucleotides. In another embodiment, the unpaired nucleotide of the single-stranded overhang that is directly adjacent to the terminal nucleotide pair contains a purine base, and the terminal nucleotide pair is a G-C pair, or at least two of the last four complementary nucleotide pairs are G-C pairs. In other embodiments, the nucleotide overhang may have 1 or 2 unpaired nucleotides, and in an exemplary embodiment the nucleotide overhang may be 5′-GC-3′. In another embodiment, the nucleotide overhang is on the 3′-end of the antisense strand.

Thus, an siNA molecule can include monomers that have been modified so as to inhibit degradation, e.g., by nucleases, e.g., endonucleases or exonucleases, found in the body of a subject. These monomers are referred to herein as NRMs or Nuclease Resistance promoting Monomers or modifications. In some cases these modifications will modulate other properties of the siNA agent as well, e.g., the ability to interact with a protein, e.g., a transport protein, e.g., serum albumin, or a member of the RISC, or the ability of the first and second sequences to form a duplex with one another or to form a duplex with another sequence, e.g., a target molecule.

While not wishing to be bound by theory, it is believed that modifications of the sugar, base, and/or phosphate backbone in an siNA agent can enhance endonuclease and exonuclease resistance and can enhance interactions with transporter proteins and one or more of the functional components of the RISC complex. In some embodiments, the modification may increase exonuclease and endonuclease resistance and thus prolong the half-life of the siNA agent prior to interaction with the RISC complex, but at the same time does not render the siNA agent inactive with respect to its intended activity as a target mRNA cleavage directing agent. Again, while not wishing to be bound by any theory, it is believed that placement of the modifications at or near the 3′- and/or 5′-end of antisense strands can result in siNA agents that meet the preferred nuclease resistance criteria delineated above.

Modifications that can be useful for producing siNA agents that exhibit the nuclease resistance criteria delineated above may include one or more of the following chemical and/or stereochemical modifications of the sugar, base, and/or phosphate backbone, it being understood that the art discloses other methods as well than can achieve the same result:

(i) chiral (Sp) thioates. An NRM may include nucleotide dimers with an enriched or pure for a particular chiral form of a modified phosphate group containing a heteroatom at the nonbridging position, e.g., Sp or Rp, at the position X, where this is the position normally occupied by the oxygen. The atom at X can also be S, Se, Nr₂, or Br₃. When X is S, enriched or chirally pure Sp linkage is preferred. Enriched means at least 70, 80, 90, 95, or 99% of the preferred form.

(ii) attachment of one or more cationic groups to the sugar, base, and/or the phosphorus atom of a phosphate or modified phosphate backbone moiety. In some embodiments, the may include monomers at the terminal position derivatized at a cationic group. As the 5′-end of an antisense sequence should have a terminal —OH or phosphate group this NRM is preferably not used at the 5′-end of an antisense sequence. The group should preferably be attached at a position on the base which minimizes interference with H bond formation and hybridization, e.g., away form the face that interacts with the complementary base on the other strand, e.g., at the 5′-position of a pyrimidine or a 7-position of a purine.

(iii) nonphosphate linkages at the termini. In some embodiments, the NRMs include non-phosphate linkages, e.g., a linkage of 4 atoms, which confers greater resistance to cleavage than does a phosphate bond. Examples include 3′ CH2-NCH₃—O—CH₂-5′ and 3′ CH₂—NH—(O.dbd.)-CH₂-5′;

(iv) 3′-bridging thiophosphates and 5′-bridging thiophosphates. In certain embodiments, the NRMs can included these structures;

(v) L-RNA, 2′-5′ linkages, inverted linkages, a-nucleosides. In certain embodiments, the NRMs include L-nucleosides and dimeric nucleotides derived from L-nucleosides; 2′-5′-phosphate, non-phosphate, and modified phosphate linkages (e.g., thiophosphates, phosphoramidates and boronophpoosphates); dimers having inverted linkages, e.g., 3′-3′- or 5′-5′-linkages; monomers having an α-linkage at the 1′-site on the sugar, e.g., the structures described herein having an α-linkage;

(vi) conjugate groups. In certain embodiments, the NRMs can include, e.g., a targeting moiety or a conjugated ligand described herein conjugated with the monomer, e.g., through the sugar, base, or backbone;

(vi) abasic linkages. In certain embodiments, the NRMs can include an abasic monomer, e.g., an abasic monomer as described herein (e.g., a nucleobaseless monomer); an aromatic or heterocyclic or polyheterocyclic aromatic monomer as described herein; and

(vii) 5′-phosphonates and 5′-phosphate prodrugs. In certain embodiments, the NRMs include monomers, preferably at the terminal position, e.g., the 5′ position, in which one or more atoms of the phosphate group is derivatized with a protecting group, which protecting group or groups are removed as a result of the action of a component in the subject's body, e.g., a carboxyesterase or an enzyme present in the subject's body. For example, a phosphate prodrug in which a carboxy esterase cleaves the protected molecule resulting in the production of a thioate anion which attacks a carbon adjacent to the 0 of a phosphate and resulting in the production of an unprotected phosphate.

“Ligand,” as used herein, means a molecule that specifically binds to a second molecule, typically a polypeptide or portion thereof such as a carbohydrate moiety, through a mechanism other than an antigen-antibody interaction. The term encompasses, for example, polypeptides, peptides, and small molecules, either naturally occurring or synthesized, including molecules whose structure has been invented by man. Although the term is frequently used in the context of receptors and molecules with which they interact and that typically modulate their activity (e.g., agonists or antagonists), the term as used herein applies more generally.

One or more different NRM modifications can be introduced into an siNA agent or into a sequence of a siRNA agent. An NRM modification can be used more than once in a sequence or in an siRNA agent. As some NRMs interfere with hybridization, the total number incorporated should be such that acceptable levels of siNA agent duplex formation are maintained.

In some embodiments, NRM modifications are introduced into the terminal cleavage site or in the cleavage region of a sequence (a sense strand or sequence) that does not target a desired sequence or gene in the subject.

In most cases, the nuclease-resistance promoting modifications will be distributed differently, depending on whether the sequence will target a sequence in the subject (often referred to as an antisense sequence) or will not target a sequence in the subject (often referred to as a sense sequence). If a sequence is to target a sequence in the subject, modifications that interfere with or inhibit endonuclease cleavage should not be inserted in the region which is subject to RISC mediated cleavage, e.g., the cleavage site or the cleavage region (as described in Elbashir et al., Genes and Dev. 15:188, 2001). Cleavage of the target occurs about in the middle of a 20 or 21 nt guide RNA, or about 10 or 11 nucleotides upstream of the first nucleotide that is complementary to the guide sequence. As used herein, cleavage site refers to the nucleotide on either side of the cleavage site, on the target, or on the iRNA agent strand which hybridizes to it. Cleavage region means a nucleotide with 1, 2, or 3 nucleotides of the cleave site, in either direction.)

Such modifications can be introduced into the terminal regions, e.g., at the terminal position or with 2, 3, 4, or 5 positions of the terminus of a sequence that targets or a sequence that does not target a sequence in the subject.

### VI. THERAPEUTIC USE

Examples of cancers that can be treated using the compositions of the invention include, but are not limited to: biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Burkitt's lymphoma, Hodgkin's disease and lymphocytic lymphomas; neuroblastomas; oral cancer including squamous cell carcinoma; ovarian cancer including those arising from epithelial cells, stromal cells, germ cells, and mesenchymal cells; pancreatic cancer; prostate cancer; rectal cancer; sarcomas including leiomyosarcoma, rhabdomyo sarcoma, liposarcoma, fibro sarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer. In general, an effective amount of the one or more compositions of the invention for treating cancer will be that amount necessary to inhibit mammalian cancer cell proliferation in situ. Those of ordinary skill in the art are well schooled in the art of evaluating effective amounts of anti-cancer agents.

In some cases, the above-described treatment methods may be combined with known cancer treatment methods. The term “cancer treatment” as used herein may include, but is not limited to, chemotherapy, radiotherapy, adjuvant therapy, surgery, or any combination of these and/or other methods. Particular forms of cancer treatment may vary, for instance, depending on the subject being treated. Examples include, but are not limited to, dosages, timing of administration, duration of treatment, etc. One of ordinary skill in the medical arts can determine an appropriate cancer treatment for a subject.

The molecules of the instant invention can be used as pharmaceutical agents. Pharmaceutical agents prevent, inhibit the occurrence, or treat (alleviate a symptom to some extent, preferably all of the symptoms) of a disease state in a subject.

The negatively charged polynucleotides of the invention can be administered (e.g., RNA, DNA, or protein complex thereof) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art and include considerations such as toxicity and forms that prevent the composition or formulation from exerting its effect.

In some embodiments, the molecules of the instant invention are administered locally to a localized region of a subject, such as a tumor, via local injection.

By “systemic administration” it is meant in vivo systemic absorption or accumulation of drugs in the blood stream followed by distribution throughout the entire body. Administration routes that lead to systemic absorption include, without limitations: intravenous, subcutaneous, intraperitoneal, inhalation, oral, intrapulmonary and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By “pharmaceutically acceptable formulation,” it is meant, a composition or formulation that allows for the effective distribution of the nucleic acid molecules of the instant invention in the physical location most suitable for their desired activity.

Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) that can enhance entry of drugs into various tissues, for example, the CNS (Jolliet-Riant and Tillement, Fundam. Clin. Pharmacol. 13:16-26, 1999); biodegradable polymers, such as poly (DL-lactide-coglycolide) microspheres for sustained release delivery after implantation (Emerich, D. F. et al., Cell Transplant 8:47-58, 1999) (Alkermes, Inc., Cambridge, Mass.); and loaded nanoparticles, such as those made of polybutylcyanoacrylate, which can deliver drugs across the blood brain barrier and can alter neuronal uptake mechanisms (Schroeder, U., et al., Prog. Neuropsychopharmacol. Biol. Psychiatry 23:941-949, 1999). Other non-limiting examples of delivery strategies, including CNS delivery of the nucleic acid molecules of the instant invention, include material described in Boado et al., J. Pharm. Sci. 87:1308-1315, 1998; Tyler et al., FEBS Lett. 421:280-284, 1999; Pardridge et al., PNAS USA. 92:5592-5596, 1995; Boado R. J., “Antisense Drug Delivery Through the Blood-Brain Barrier,” Adv. Drug Del. Rev. 15:73-107, 1995; Aldrian-Herrada et al., Nucleic Acids Res. 26:4910 4916, 1998; and Tyler et al., PNAS USA 96:7053-7058, 1999. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al., Chem. Rev. 95:2601-2627, 1995; Ishiwata et al., Chem. Pharm. Bull. 43:1005-1011, 1995). Such liposomes have been shown to accumulate selectively in tumors, presumably by extravasation and capture in the neovascularized target tissues (Lasic et al., Science 267:1275-1276, 1995; Oku et al., Biochim. Biophys. Acta 1238:86-90, 1995). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 42:24864-24870, 1995; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The invention also includes compositions comprising interfering nanoparticles composed of natural amino acids labeled with lipids and complexed with unmodified or modified siRNA as described in Baigude, H. et al., ACS Chem. Biol. 2:237-241, 2007, which is hereby incorporated by reference herein.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art and are described, for example, Remington's Pharmaceutical Sciences, Mack Publishing Co. (A. R. Gennaro, ed., 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate, or kaolin, or as soft gelatin capsules wherein the active ingredient is mixed with water or an oil medium, for example, peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth, and gum acacia; dispersing or wetting agents can be a naturally-occurring phosphatide, for example, lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and an hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example, ethyl or n-propyl p-hydroxybenzoate, one or more coloring agents, one or more flavoring agents, and one or more sweetening agents such as sucrose or saccharin.

Oily suspensions can be formulated by suspending the active ingredients in a vegetable oil, for example. arachis oil, olive oil, sesame oil or coconut oil, or in a mineral oil such as liquid paraffin. The oily suspensions can contain a thickening agent, for example, beeswax, hard paraffin, or cetyl alcohol. Sweetening agents and flavoring agents can be added to provide palatable oral preparations. These compositions can be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring, and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example, gum acacia or gum tragacanth, naturally-occurring phosphatides, for example, soy bean, lecithin, and esters or partial esters derived from fatty acids and hexitol, anhydrides, for example, sorbitan monooleate, and condensation products of the said partial esters with ethylene oxide, for example, polyoxyethylene sorbitan monooleate. The emulsions can also contain sweetening and flavoring agents.

Syrups and elixirs can be formulated with sweetening agents, for example, glycerol, propylene glycol, sorbitol, glucose, or sucrose. Such formulations can also contain a demulcent, a preservative, and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 mg per patient or subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient or subject depends upon a variety of factors including the activity of the specific compound employed, the age, body weight, general health, sex, diet, time of administration, route of administration, and rate of excretion, drug combination, and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

Examples are provided below to further illustrate different features and advantages of the present invention. The examples also illustrate useful methodology for practicing the invention. These examples should not be construed to limit the claimed invention.

### Example 1

This example shows that miR-210 is upregulated under hypoxic conditions in tumor cell lines.

Methods.

Cell Cultures. HCT116 Dicer^{ex5}, RKO Dicer^{ex5} and DLD-1 Dicer^{ex5} cells were previously described (Cummins et al., 2006). Wild type HCT116, RKO, DLD-1, HeLa, A549, MCF7, Hep3B, HuH7, H1299, U251, and ME-180 cells were from the American type Culture Collection, Rockville, Md. 786-O renal cell carcinoma cells and their variants were a kind gift from William G. Kaelin. 786-O WT7 cells were 786-O stably transfected with pRc-CMV-HA-VHL (WT7) (von Hippel-Lindau (“VHL”) gene functional) (Li et al., 2007), 786-OpBABE cells were 786-O infected with pBABE empty vector (and therefore VHL defective), and 786-O-pBABE-VHL cells were 786-O infected with pBABE-VHL (and therefore VHL functional). HFF-pBABE and HFF-Myc were described previously (Benanti et al., 2007).

Hypoxia. Hypoxia (1% O₂) was achieved using a HERAcell 150 Tri-Gas cell culture incubator (Kendro Laboratories Products, Newtown, Conn.) or an In Vivo2 200 hypoxic station (Ruskinn Technologies).

Quantitative PCR. miRNA levels were determined using a quantitative primer-extension PCR assay (Raymond et al., 2005). Ct values were converted to copy numbers by comparison to standard curves generated using single stranded mature miRNAs and are expressed as copies/10 pg input RNA (approximately equivalent to copies/cell).

Microarray analysis. Microarray analysis was performed as described (Jackson et al., “Expression Profiling Reveals Off-Target Gene Regulation by RNAi,” Nat. Biotechnol. 21:635-637, 2003). Briefly, total RNA was purified by a QIAGEN RNeasy kit and processed as described previously (Hughes et al., “Expression Profiling Using Microarrays Fabricated by an Ink-Jet Oligonucleotide Synthesizer,” Nat. Biotechnol. 19:342-347, 2001) for hybridization to microarrays containing oligonucleotides corresponding to about 21,000 human genes. Ratio hybridizations were performed with fluorescent label reversal to eliminate dye bias. The data shown are signature genes that display a difference in expression level (p<0.01) relative to mock-transfected cells. No cut offs were placed on fold change in expression. The data were analyzed using Rosetta Resolver™ software. Differences in transcript regulation between unmodified and modified duplexes were calculated individually for each transcript. Transcript regulation was calculated as the error-weighted mean log₁₀ ratio for each transcript across the fluor-reversed pair. Differences in regulation between unmodified and modified duplex were then divided by the log₁₀ ratio for the unmodified duplex for that transcript to result in the normalized mean log ratio change.

Results

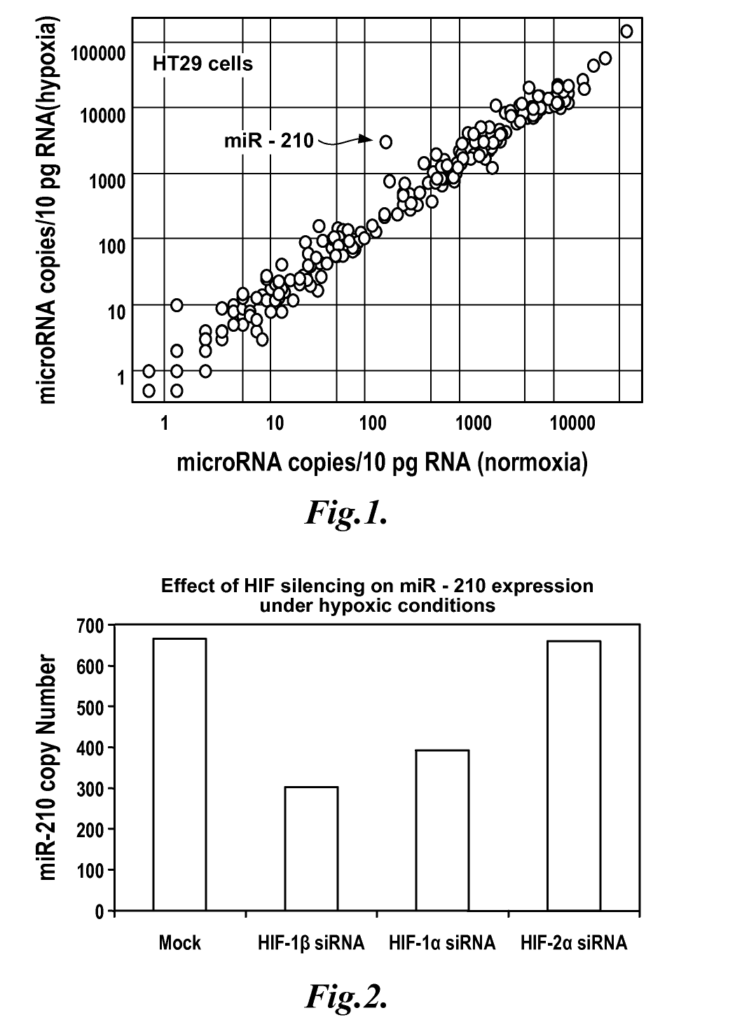

To discover microRNAs that are modulated during the hypoxia response in tumors, the expression levels of approximately 200 microRNAs in a panel of 8 cancer cell lines from 4 different tissues was determined. The cell lines tested were HCT116, HT29, DLD1, and RKO (colon); HeLa and ME-180 (cervical); U251 (glioma), and 786-O (kidney). HMEC (human mammary epithelial cell) and HFF (human foreskin fibroblasts) cells were included as normal cell controls. Cells were exposed to normoxia (21% O₂) or hypoxia (1% O₂) for 24 hours and RNAs were isolated and analyzed for the expression of microRNAs using primer extension quantitative PCR (PE-qPCR). As shown in FIG. 1, miR-210 was up-regulated approximately 19-fold in HT29 cells after hypoxia treatment (microRNA copy number per 10 pg of RNA increased from 165 to 3075). Most of the other miRNAs fall on the diagonal of the graph, indicating similar expression levels between Normoxia (X axis) and Hypoxia (Y axis).

Previous work showed that miR-210 was among several microRNAs induced by hypoxia (Kulshreshtha et al., 2007a). Table 2 shows that miR-210 was upregulated more than any other miRNA tested in HT129 cells.

TABLE 2
Upregulation of miRNAs Under Hypoxic
Conditions in HT29 cells.^{a}

| miRNA | Normoxia | Hypoxia | Fold Induction |
|---|---|---|---|
| miR-210 | 165 | 3075 | 18.62 |
| miR-302b | 1 | 10 | 6.97 |
| miR-135a | 32 | 161 | 4.95 |
| miR-374 | 2441 | 10926 | 4.48 |
| miR-130b | 181 | 776 | 4.29 |
| miR-146 | 23 | 90 | 3.93 |
| let7f | 5480 | 20527 | 3.75 |
| miR-142-3p | 560 | 1952 | 3.49 |
| miR-30a-5p | 422 | 1450 | 3.44 |
| miR-15a | 1221 | 4183 | 3.43 |
| miR-433 | 13 | 42 | 3.23 |
| miR-450 | 9 | 28 | 3.06 |
| miR-30d | 1667 | 5007 | 3.00 |
| miR-223 | 9 | 25 | 2.95 |
| miR-380-5p | 5 | 15 | 2.94 |
| miR-365 | 1405 | 4051 | 2.88 |
| miR-150 | 51 | 146 | 2.84 |
| let7g | 3062 | 8592 | 2.81 |
| miR-9 | 1 | 2 | 2.78 |
| miR-152 | 35 | 96 | 2.77 |
| miR-30c | 1055 | 2901 | 2.75 |
| miR-296 | 3 | 9 | 2.73 |
| miR-375 | 260 | 701 | 2.69 |
| miR-217 | 5 | 13 | 2.64 |
| miR-106b | 3431 | 8874 | 2.59 |
| miR-27a | 4569 | 11782 | 2.58 |
| miR-16 | 4335 | 11093 | 2.56 |
| miR-151 | 59526 | 147639 | 2.48 |
| miR-126 | 4 | 10 | 2.48 |
| miR-25 | 2076 | 5021 | 2.42 |
| miR-190 | 58 | 139 | 2.39 |
| miR-135b | 680 | 1610 | 2.37 |
| miR-148a | 25 | 60 | 2.36 |
| miR-345 | 47 | 107 | 2.30 |
| miR-429 | 6865 | 15650 | 2.28 |
| miR-34c | 1 | 2 | 2.27 |
| miR-181c | 45 | 100 | 2.23 |
| miR-15b | 989 | 2191 | 2.22 |
| miR-26a | 3488 | 7578 | 2.17 |
| miR-337 | 0 | 1 | 2.15 |
| miR-30e-3p | 65 | 138 | 2.13 |
| miR-200c | 7081 | 14962 | 2.11 |
| miR-224 | 1465 | 3065 | 2.09 |
| miR-181b | 1534 | 3192 | 2.08 |
| let7i | 10944 | 22427 | 2.05 |
| miR-367 | 1 | 2 | 2.04 |
| miR-28 | 53 | 108 | 2.02 |
| miR-34a | 635 | 1283 | 2.02 |
| miR-183 | 530 | 1053 | 1.99 |
| miR-32 | 252 | 495 | 1.96 |
| miR-129 | 2 | 4 | 1.91 |
| miR-10a | 11115 | 21123 | 1.90 |
| miR-371 | 1 | 2 | 1.90 |
| miR-24 | 1041 | 1974 | 1.90 |
| miR-184 | 5 | 9 | 1.90 |
| miR-31 | 10994 | 20719 | 1.88 |
| miR-96 | 732 | 1377 | 1.88 |
| miR-340 | 12 | 23 | 1.88 |
| miR-133a | 11 | 21 | 1.88 |
| miR-202 | 1 | 1 | 1.87 |
| miR-10b | 2194 | 4075 | 1.86 |
| miR-216 | 12 | 22 | 1.84 |
| miR-323 | 0 | 0 | 1.80 |
| miR-103 | 5829 | 10469 | 1.80 |
| miR-133b | 8 | 14 | 1.79 |
| miR-205 | 1567 | 2806 | 1.79 |
| miR-212 | 6 | 11 | 1.79 |
| miR-302b* | 1 | 2 | 1.78 |
| miR-147 | 4 | 8 | 1.78 |
| miR-26b | 4736 | 8368 | 1.77 |
| miR-140 | 252 | 443 | 1.76 |
| miR-192 | 2642 | 4603 | 1.74 |
| miR-125b | 10 | 18 | 1.74 |
| miR-34b | 30 | 52 | 1.71 |
| miR-132 | 44 | 75 | 1.71 |
| miR-302c | 7 | 13 | 1.71 |
| let7e | 5534 | 9427 | 1.70 |
| miR-372 | 1 | 1 | 1.70 |
| miR-221 | 26400 | 43924 | 1.66 |
| miR-29a | 5989 | 9904 | 1.65 |
| miR-197 | 297 | 490 | 1.65 |
| miR-182 | 768 | 1259 | 1.64 |
| miR-21 | 35242 | 57757 | 1.64 |
| miR-122a | 1 | 1 | 1.63 |
| miR-30b | 8761 | 13902 | 1.59 |
| miR-196a | 464 | 734 | 1.58 |
| miR-338 | 1082 | 1705 | 1.58 |
| miR-206 | 3 | 4 | 1.58 |
| miR-215 | 14165 | 21881 | 1.54 |
| miR-425 | 52 | 80 | 1.54 |
| miR-145 | 5 | 7 | 1.53 |
| miR-376a | 3 | 4 | 1.53 |
| miR-301 | 937 | 1427 | 1.52 |
| miR-19b | 11003 | 16720 | 1.52 |
| miR-22 | 1770 | 2690 | 1.52 |
| let7d | 10657 | 16146 | 1.52 |
| miR-193 | 26 | 40 | 1.51 |
| miR-185 | 6590 | 9874 | 1.50 |
| miR-29c | 1881 | 2788 | 1.48 |
| miR-93 | 4386 | 6358 | 1.45 |
| miR-452 | 67 | 96 | 1.44 |
| miR-125a | 4046 | 5824 | 1.44 |
| miR-189 | 89 | 128 | 1.43 |
| miR-27b | 2709 | 3884 | 1.43 |
| miR-222 | 2802 | 4003 | 1.43 |
| miR-422a | 3 | 4 | 1.42 |
| miR-329 | 13 | 18 | 1.42 |
| miR-182* | 9 | 12 | 1.42 |
| miR-200a | 12611 | 17735 | 1.41 |
| miR-23a | 7199 | 10064 | 1.40 |
| miR-203 | 843 | 1176 | 1.39 |
| miR-191 | 1045 | 1452 | 1.39 |
| miR-331 | 375 | 521 | 1.39 |
| miR-194 | 3091 | 4290 | 1.39 |
| miR-376b | 6 | 8 | 1.38 |
| miR-302a | 160 | 219 | 1.37 |
| miR-181a | 1404 | 1920 | 1.37 |
| miR-302d | 59 | 80 | 1.36 |
| miR-423 | 640 | 860 | 1.34 |
| miR-141 | 5276 | 7075 | 1.34 |
| miR-20 | 7315 | 9796 | 1.34 |
| miR-7 | 20400 | 27271 | 1.34 |
| miR-324-5p | 251 | 335 | 1.34 |
| miR-127 | 33 | 44 | 1.33 |
| miR-320 | 1306 | 1724 | 1.32 |
| miR-1 | 4 | 5 | 1.32 |
| miR-149 | 75 | 99 | 1.32 |
| miR-339 | 960 | 1257 | 1.31 |
| miR-412 | 2 | 3 | 1.31 |
| miR-17-5p | 2469 | 3213 | 1.30 |
| miR-372 | 1 | 1 | 1.29 |
| miR-214 | 22 | 28 | 1.27 |
| miR-23b | 6081 | 7744 | 1.27 |
| miR-211 | 4 | 5 | 1.25 |
| miR-148b | 667 | 833 | 1.25 |
| miR-134 | 23 | 29 | 1.25 |
| miR-98 | 1743 | 2151 | 1.23 |
| miR-220 | 0 | 0 | 1.23 |
| miR-213 | 56 | 67 | 1.20 |
| miR-99b | 1616 | 1934 | 1.20 |
| miR-18 | 6073 | 7232 | 1.19 |
| let7c | 2681 | 3161 | 1.18 |
| miR-200b | 8944 | 10519 | 1.18 |
| miR-342 | 0 | 0 | 1.17 |
| miR-204 | 3 | 3 | 1.15 |
| miR-328 | 307 | 353 | 1.15 |
| miR-330 | 49 | 56 | 1.15 |
| miR-92 | 10712 | 12266 | 1.15 |
| miR-363 | 1 | 1 | 1.14 |
| miR-107 | 2176 | 2462 | 1.13 |
| miR-422b | 220 | 248 | 1.13 |
| miR-195 | 14 | 16 | 1.12 |
| miR-143 | 39 | 43 | 1.12 |
| miR-30e-5p | 925 | 1033 | 1.12 |
| miR-218 | 1 | 1 | 1.12 |
| miR-188 | 22 | 24 | 1.11 |
| miR-138 | 2 | 3 | 1.10 |
| miR-99a | 52 | 57 | 1.10 |
| miR-198 | 83 | 91 | 1.09 |
| miR-106a | 2082 | 2262 | 1.09 |
| miR-199a | 72 | 78 | 1.08 |
| miR-100 | 834 | 900 | 1.08 |
| miR-370 | 97 | 104 | 1.07 |
| miR-29b | 10286 | 10835 | 1.05 |
| miR-377 | 24 | 24 | 1.04 |
| miR-19a | 11203 | 11653 | 1.04 |
| miR-153 | 5 | 5 | 1.03 |
| miR-324-3p | 858 | 887 | 1.03 |
| let7a | 12686 | 13103 | 1.03 |
| miR-199a* | 12 | 13 | 1.03 |
| miR-381 | 74 | 76 | 1.02 |
| miR-199b | 2 | 2 | 1.02 |
| miR-326 | 129 | 131 | 1.02 |
| miR-101 | 655 | 663 | 1.01 |
| let7b | 13014 | 13171 | 1.01 |
| miR-302a* | 3 | 3 | 0.98 |
| miR-187 | 2 | 2 | 0.98 |
| miR-128b | 299 | 280 | 0.94 |
| miR-196b | 355 | 332 | 0.94 |
| miR-299 | 2 | 2 | 0.92 |
| miR-346 | 73 | 67 | 0.92 |
| miR-186 | 1849 | 1693 | 0.92 |
| miR-219 | 78 | 70 | 0.91 |
| miR-128a | 869 | 761 | 0.88 |
| miR-378 | 34 | 27 | 0.79 |
| miR-325 | 26 | 20 | 0.78 |
| miR-302c* | 508 | 379 | 0.75 |
| miR-448 | 13 | 8 | 0.65 |
| miR-144 | 7 | 4 | 0.62 |
| miR-154* | 1 | 0 | 0.59 |
| miR-124a | 31 | 17 | 0.54 |
| miR-451 | 8 | 3 | 0.45 |
| miR-154 | 2 | 1 | 0.44 |

^{a}Copies per 10 pg of RNA of the miRNAs were determined using primer extension quantitative PCR (PE-qPCR). delta Ct was converted to copy number by comparison with standard curves generated by use of defined input of single stranded mature miRNAs.

Further, as shown in Table 3, hypoxia treatment results in increased expression of miR-210 in most of the cell lines tested.

TABLE 3
Upregulation of miR-210 by hypoxia in tumor cell lines.*

| | Fold induction |
|---|---|
| Cell Line | (hypoxia/normoxia) |
| HeLa | 27.2 |
| ME180 | 9.1 |
| HCT116 | 5.4 |
| HT29 | 18.6 |
| DLD1 | 9.1 |
| RKO | 16.9 |
| U251 | 2.1 |
| 786-O-pBABE-VHL | 3.7 |
| HMEC | 1.8 |
| HFF-pBABE | 8.6 |
| HFF-c-Myc | 7.6 |

*Fold induction of miR-210 by hypoxia relative to normoxia (copy number per 10 pg RNA determined by PE-qPCR).

miR-210 is located on Chromosome 11 in the intron of a non-coding transcriptional unit (Genbank Accession number AK123483). Transcription from the miR-210 promoter yields the pri-miR-210 primary transcript (SEQ ID NO:2), which is processed in the cell to produce the mature microRNA (SEQ ID NO:1) (Kim, V. N., and J. W. Nam, “Genomics of microRNA,” Trends Genet. 22:165-173, 2006). Expression of the primary pri-miR-210 transcript was detected by microarray analysis as described (Jackson, A. L., et al., “Expression Profiling Reveals Off-Target Gene Regulation by RNAi,” Nat. Biotechnol. 21:635-637, 2003). As shown in Table 4, the expression of the pri-miR-210 primary transcript is also upregulated under hypoxic conditions in the following cell lines: HCT116, HeLa, HT29, U251, H1299 (human lung carcinoma), MCF7 (human breast adenocarcinoma), A549 (human lung epithelial carcinoma), PC-3 (human prostate adenocarcinoma), Hep3B (human hepatoma), HuH7 (human hepatoma), DLD1, RKO, HFF, and ME-180. Further, as shown in Table 5, a time course study revealed that miR-210 up-regulation was detected as early as 4 hours after the start of hypoxia treatment in ME-180 cells, indicating that miR-210 might be a direct target of HIF.

TABLE 4
Upregulation of pri-miR-210 by Hypoxia in Tumor Cell Lines.*

| | Fold induction | |
|---|---|---|
| Cell Line | (hypoxia/normoxia) | p-value |
| HCT116 | 5.9 | 1.18E−27 |
| HeLa | 16.4 | 1.38E−35 |
| HT29 | 8.6 | 5.56E−33 |
| U251 | 3.1 | 4.16E−14 |
| 786-O-pBABE-VHL | 2.5 | 6.26E−12 |
| H1299 | 10.0 | 2.21E−24 |
| MCF7 | 8.2 | 5.67E−32 |
| A549 | 12.4 | 2.56E−27 |
| PC-3 | 3.3 | 1.42E−10 |
| Hep3B | 5.2 | 1.04E−24 |
| HuH7 | 9.5 | 4.10E−36 |
| DLD1 | 2.3 | 1.51E−08 |
| RKO | 1.6 | 0.003791 |
| HFF-pBABE | 1.9 | 0.000076 |
| HFF-Myc | 1.3 | 0.061481 |

*The level of pri-miR-210 was determined by gene expression profiling in cells exposed to normoxia or hypoxia for 24 hours. Fold induction of pri-miR-210 by hypoxia relative to normoxia.

TABLE 5
Time Course of Upregulation of pri-
miR-210 by Hypoxia in ME180 Cells.*

| | Fold induction | |
|---|---|---|
| Time | (hypoxia/normoxia) | p-value |
| 4 hr | 10.2 | 9.55E−33 |
| 8 hr | 19.4 | 1.82E−38 |
| 24 hr | 18.9 | 2.79E−25 |

*The level of pri-miR-210 was determined by gene expression profiling in cells exposed to normoxia or hypoxia for 4, 8 and 24 hours. Fold induction of pri-miR-210 by hypoxia relative to normoxia.

In summary, this example shows that miR-210 is up-regulated under hypoxic conditions in tumor cell lines and primary cell lines, suggesting that upregulation of miR-210 is a universal physiological response to the hypoxic environment. Further, miR-210 is the most highly induced microRNA in HT29 cells treated with hypoxia. The early time course of induction by hypoxia suggests that pri-miR-210 is a direct target of HIF transcription factors.

### Example 2

This example shows that miR-210 is directly regulated by HIF-1 and HIF-2.

Methods

siRNA duplexes. siRNA sequences that target HIF-1α, HIF-1β, and HIF-2α were designed with an algorithm developed to increase efficiency of the siRNAs for silencing while minimizing their “off target” effects (Jackson et al., 2003b). siRNA duplexes were ordered from Sigma-Proligo (Boulder, Colo.).

Transfections. Cells were plated 24 hours prior to transfection. Cells were transfected in 6-well plates using Lipofectamine RNAiMAX (Invitrogen, Carlsbad, Calif.). siRNAs were used at 100 nM final concentration. For HIF-1α, HIF-1β, and HIF-2α siRNA experiments, three siRNAs targeting the same gene were pooled at equal molarity (final concentration of each siRNA 33 nM; total siRNA concentration, 100 nM). The siRNAs targeting HIF-1α comprise the guide sequences of SEQ ID NOs: 6-8; the siRNAs targeting HIF-1β comprise the guide sequences of SEQ ID NOs: 9-11; and the siRNAs targeting HIF-2α comprise the guide sequences of SEQ ID NOs: 12-14.

ChIP assay. Chromatin IP was performed following the protocol from Genpathway (San Diego, Calif.). Cells were exposed to hypoxia (1% O₂) or normoxia (21% O₂) for 24 hours and fixed with 1% freshly-prepared formaldehyde for 15 minutes at room temperature. Nuclear extracts were prepared and sonicated to produce DNA fragments. Antibody against HIF-1α (Abcam: ab2185) was used for immunoprecipitation. Binding events of HIF-1α antibody to the miR210 promoter regions were determined by quantitative PCR (Q-PCR). Q-PCR reactions were carried out in triplicate on specific genomic regions using SYBR Green Supermix (Bio-Rad). Experimental Ct values were converted to copy numbers detected by comparison with a DNA standard curve run on the same PCR plate. Copy number values were then normalized for primer efficiency by dividing by the values obtained using Input DNA and the same primer pairs. Forward primer (5′-AGGAGCCTTGACGGTTTGAC-3′) (SEQ ID NO:21) and reverse primer (5′-GAGGACCAGGGTGACAGTG-3′) (SEQ ID NO:22) were used to amplify the promoter region of miR-210 with the putative HIF binding site. The lactate dehydrogenase A (LDHA) promoter HIF-1α binding site served as the positive binding control. A region of genomic DNA without HIF-1α binding sites served as the negative binding control.

Results

In the first set of experiments, HCT116 or 786-O cells were transfected with siRNAs that inhibit expression of HIF-1α, HIF-2α, or the common subunit HIF-1β. Twenty-four (24) hours after transfection the cells were exposed to hypoxia for another 24 hours. Gene expression profiles were then determined by microarray analysis. Silencing of HIF-1α in HCT116 cells using equal amounts of three siRNAs (SEQ ID NOs:6, 7, and 8) reduced the expression of pri-miR-210 transcript (SEQ ID NO:2) by 64%. Silencing of HIF-1β in HCT116 cells using equal amounts of three siRNAs (SEQ ID NOs:9, 10, and 11) decreased expression of pri-miR-210 transcript by 76%. In 786-O-pBABE cells (786-O cells infected with pBABE empty vector, and therefore VHL defective) that are primarily HIF-2α dependent for HIF activity, silencing of HIF-2α using equal amounts of three siRNAs (SEQ ID NOs:12, 13, and 14) reduced expression of pri-miR-210 by 45%. Silencing of HIF-1β in 786-O-pBABE cells using equal amounts of three siRNAs (SEQ ID NOs:9, 10, and 11) reduced expression of pri-miR-210 by 64%.

In contrast, overexpression of a stabilized variant of HIF-2α (which is not recognized by the VHL complex for degradation) in 786-O-WT7 cells (a stable subclone of 786-O cells expressing wild-type pVHL) induced expression of pri-miR-210 3.37-fold, consistent with miR-210 being a transcriptional target of HIF.

In the second set of experiments, the effect of HIF silencing on the expression of mature miR-210 (SEQ ID NO:1) in HCT116 Dicer^{ex5} cells after hypoxia treatment was determined. HCT116 Dicer^{ex5} cells are homozygous for a mutation in the Dicer helicase domain and have negligible background of endogenous microRNAs (Cummins et al., 2006; Linsley et al., 2007). As shown in FIG. 2, silencing of HIF-1α reduced miR-210 copy number by 30%, whereas silencing of HIF-1β reduced miR-210 copy number by 45%. Inhibiting HIF-2α had no effect because HIF-2α is not the dominant HIF a family member in the HCT116 cell line.

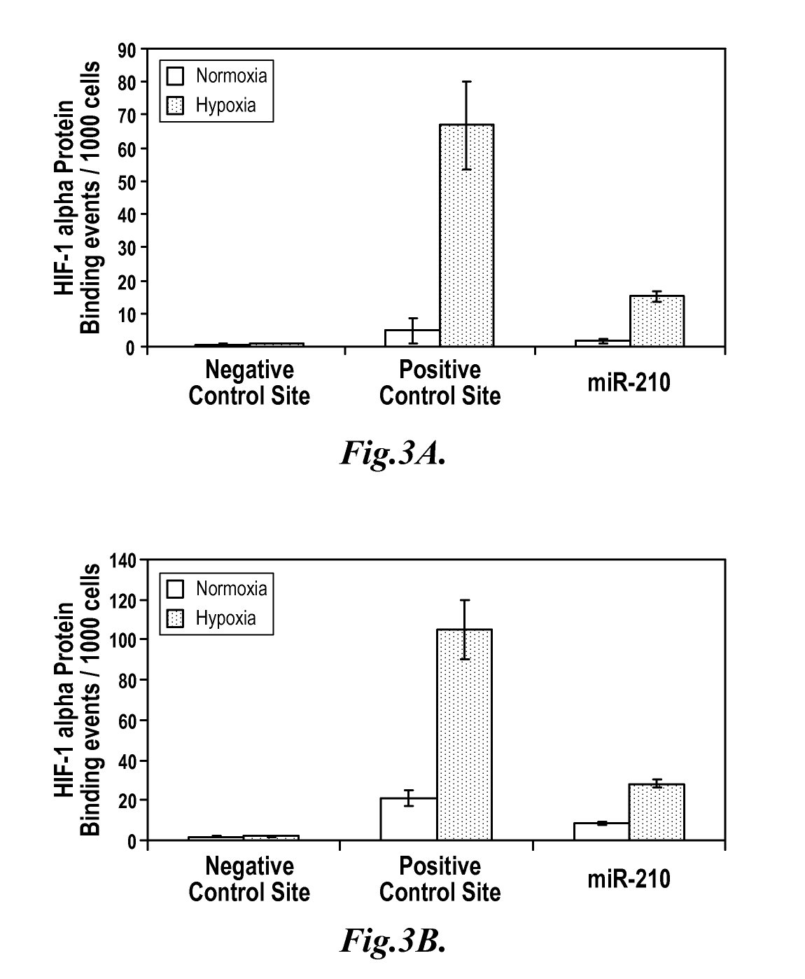

To confirm that miR-210 was a direct target of HIF, chromatin immunoprecipitation assays were performed. As shown in FIGS. 3A and 3B, HIF-1α antibody immunoprecipitated the postulated promoter region of miR-210 under hypoxic conditions in both HuH7 and U251 cells.

In summary, the data shown in this example indicate that miR-210 expression is directly modulated by HIF family members, in particular, HIF-1α, HIF-2α, and HIF-1β.

### Example 3

This example shows that miR-210 functions as a biomarker for metastatic potential.

Methods

RNA was isolated from matched tumor and adjacent non-involved normal tissues from the same patients. The level of pri-miR-210 was determined by microarray gene expression profiling. In one set of experiments, for each cancer type, up to 75 pairs of matched tumor and adjacent non-involved normal samples from the same patients were profiled against a pool of a subset of the normal samples. The combined p-value indicates the probability that the expression of pri-miR-210 in normal samples is the same as that in tumor samples. The data is plotted on the Y-axis as the log 10 value of expression intensity/common reference (a universal human RNA sample).

In the second set of experiments, RNA was isolated from a series of 29 tumors (9 breast, 5 lung, 5 gastric, 5 kidney, and 5 colon cancer tumors) and 28 adjacent non-involved normal tissues. mRNA expression was measured using microarrays and miR-210 levels were determined using a quantitative primer-extension PCR assay as described by Raymond et al. (2005). mRNA and miRNA expression levels in tumor and adjacent normal tissues are expressed as ratios to a pool of normal samples from each tissue type. Correlations were calculated between the expression ratios in tumor tissues and the expression ratios of miR-210 and transcripts up-regulated after 24 hours of hypoxia treatment in 21 tumor cell lines in tissue culture. As a control, correlations were also calculated for approximately 200 random permutations of expression ratios (random transcripts).

In a third set of experiments, pri-miR-210 expression was determined in a retrospective study of a group of 311 breast cancer patients from the Netherlands Cancer Institute (NM). Pri-miR-210 transcript levels from each individual primary tumor were compared to the median value determined for all tumors in the study, and the patients were classified into two groups: group one with miR-210 expression levels higher than the median (up-regulated group), and group two with miR-210 expression levels lower than the median (down-regulated group). The probability of metastasis free survival between the two groups was described by a Kaplan-Meier survival curve and compared by the log-rank test. Similar analysis was performed on 58 melanoma tumor samples collected from lymph node metastasis, of which 35 developed distant metastases.

Results

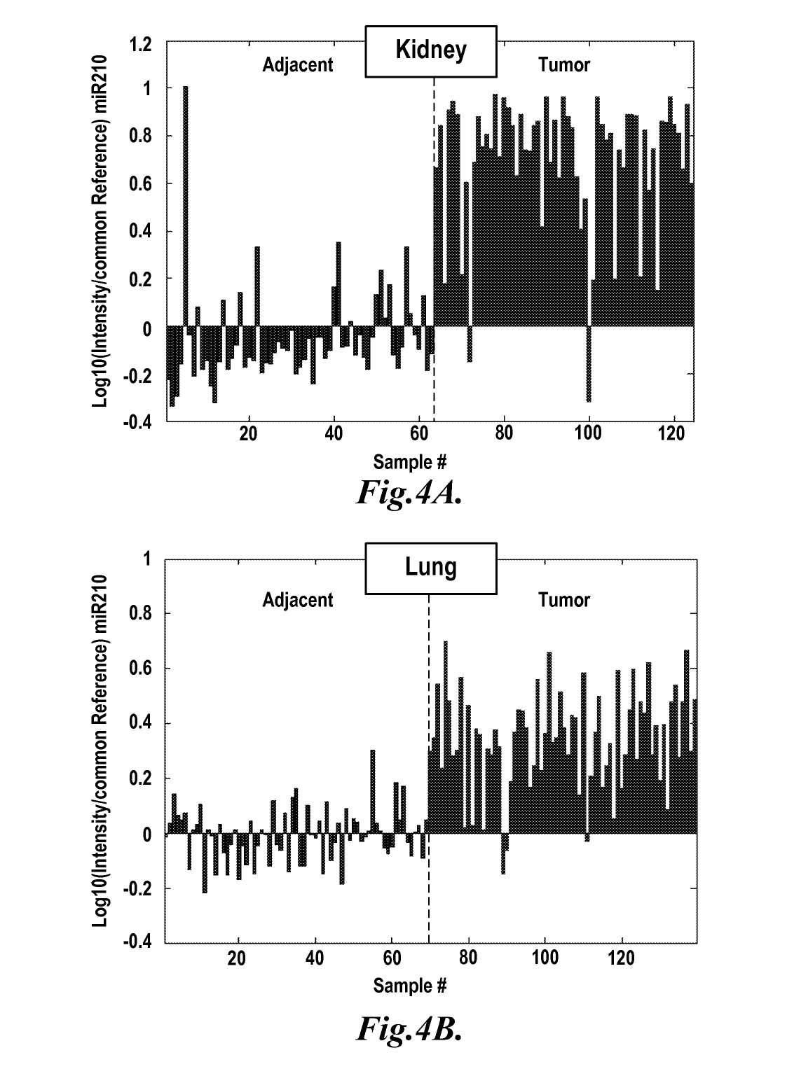

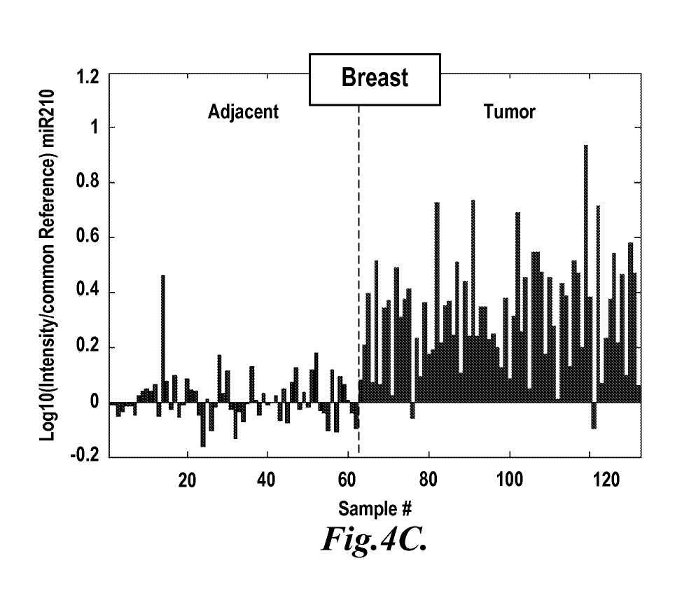

As shown in FIGS. 4A-4C, pri-miR-210 is overexpressed in a panel of human kidney, lung, and breast tumors. The differential expression of pri-miR-210 between tumors and adjacent normal tissue was significant for kidney (p-value=6.4e-34), lung (p-value=4.3e-28) and breast (p-value=4.6e-20) cancers. However, miR-210 was not upregulated in colon and gastric cancers relative to normal tissue.

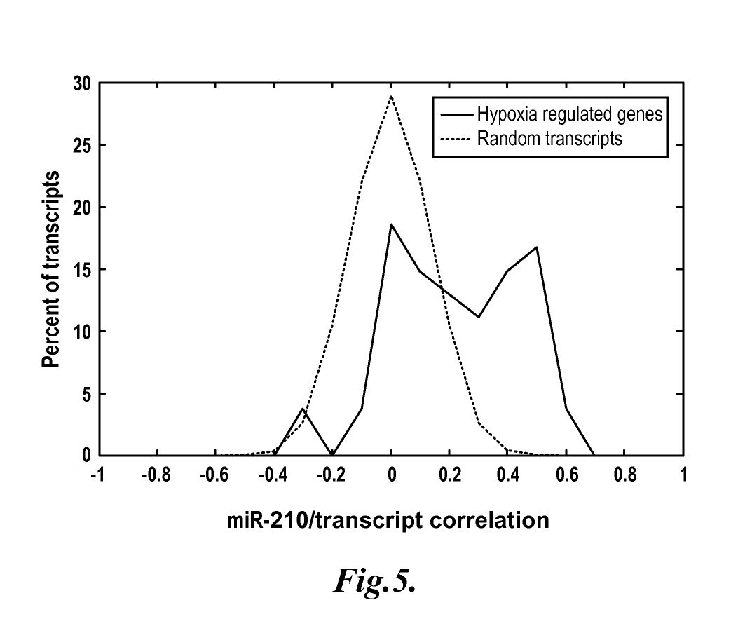

Further, as shown in FIG. 5, a significant positive correlation exists between miR-210 levels and the transcripts up-regulated by hypoxia in 29 human tumors comprising tumors from 9 breast cancer patients, 5 lung cancer patients, 5 gastric cancer patients, 5 kidney cancer patients, and 5 colon cancer patients. A total of 63 genes that were upregulated by hypoxia in tumor cell lines in culture were positively correlated with miR-210 expression ratios in tumor cells from the 29 individual tumors (p-value=1.27e-13).

To determine if miR-210 levels had predictive power for patient outcome, the level of pri-miR-210 in a set of previously studied breast cancer samples from 331 NM patients was determined (see van't Veer, L. J., et al., “Gene Expression Profiling Predicts Clinical Outcome of Breast Cancer,” Nature 415:530-536, 2002). As shown in FIG. 6A, up-regulation of pri-miR-210 was found to positively correlate with the metastatic potential (decreased probability of metastasis-free survival) of this set of breast cancer tumor samples. As shown in FIG. 6B, up-regulation of pri-miR-210 was also found to positively correlate with a decreased probability of metastasis-free survival in patients with melanoma tumors. Stated another way, the expression of miR-210 showed a significant inverse correlation with metastasis-free survival for both breast and melanoma cancer.

In summary, this example provides data indicating the miR-210 is a useful biomarker for hypoxia in certain types of cancers, particularly breast, kidney, and lung tumors. Further, the data presented in this example suggests that miR-210 may play a role in tumor growth under hypoxic conditions. This example also provides data showing the unexpected result that the level of expression of miR-210 is useful as an independent predictor of the probability of tumor cell metastasis, and therefore of cancer prognosis and patient outcomes.

### Example 4

This example shows that miR-210 overrides hypoxia induced cell-cycle arrest, and also regulates the cell-cycle under normoxia.

Methods

RNA Duplexes and Transfections. RNA duplexes corresponding to mature miRNAs were designed as described (Lim et al., 2005). miRNA duplexes were ordered from Sigma-Proligo (Boulder, Colo.). miRCURY™ LNA Knockdown probes (anti-miRs) for miR-210 (#118103-00) and miR-185 (#138529-00) were obtained from Exiqon, Copenhagen, Denmark. Cells were transfected as described in Example 2. mRNAs were transfected at 10 nM, and LNA modified anti-miRs were transfected at 200 nM.

Cell cycle analysis. Cells were seeded in 6-well plates at a density such that they would be 50-60% confluent on the day of analysis. Twenty-four hours after transfection, cells were exposed to hypoxia or normoxia for an additional 24 hours before harvesting. The supernatant from each well was combined with cells harvested from each well by trypsinization. Alternatively, Nocodazole (100 ng/ml, Sigma-Aldrich) was added 30 hours after transfection and cells were further incubated for 16 hours before harvesting. Cells were collected by centrifugation at 1200 rpm for 5 minutes; fixed with ice cold 70% ethanol for about 30 minutes; washed with PBS; and resuspended in 0.5 ml of PBS containing Propidium Iodide (10 μg/ml) and RNase A (1 mg/ml). After a final incubation at 37° C. for 30 minutes, cells were analyzed by flow cytometry using a FACSCalibur flow cytometer (Becton Dickinson). For BrdU-incorporation analysis, cells were pulsed with BrdU (BD Bioscience) for 60 minutes before harvesting. Fixed cells were stained with FITC-conjugated anti-BrdU antibody and the DNA dye 7-amino-actinomycin D (7-AAD). Data were analyzed using FlowJo software (Tree Star, Ashland, Oreg.).

Results

Hypoxia treatment has been shown to induce cell cycle arrest at the G1-S transition (Goda, N., et al., “Hypoxia-Inducible Factor 1Alpha Is Essential for Cell Cycle Arrest During Hypoxia,” Mol. Cell. Biol. 23:359-369, 2003; Gordan, J. D., et al., “HIF-2Alpha Promotes Hypoxic Cell Proliferation by Enhancing c-Myc Transcriptional Activity,” Cancer Cell 11:335-347, 2007a; Hammer, S., et al., “Hypoxic Suppression of the Cell Cycle Gene CDC25A in Tumor Cells,” Cell Cycle 6:1919-1926, 2007). To determine if miR-210 regulates cell cycle progression during hypoxia, HCT116 Dicer cells were transfected as described in Example 2 with wild-type miR-210 duplexes and a seed region mutant of miR-210 (guide strand comprising SEQ ID NO:5). As shown in Table 6, miR-210 reduced the fraction of cells in G1 (from 60% to 21%) and increased the number of cells in S phase (from 12.3% to 21.7%) under hypoxic conditions in comparison to a mock transfection control. The G2/M population also increased from 21.9% to 46.1%. In contrast, the miR-210 seed region mutant did not affect the cell cycle profiles, suggesting that the cell cycle effect of miR-210 was target specific.

TABLE 6
Cell cycle progression after transfection
with miR-210 and miR-210 mt.*

| | Cell-cycle phase | Control | miR-210 | miR-210 mt |
|---|---|---|---|---|
| Normoxia | G1 (% cells) | 45 | 19 | 50 |
| | S | 23 | 20 | 14 |
| | G2/M | 28 | 54 | 28 |
| Hypoxia | G1 | 60 | 21 | 54 |
| | S | 12 | 22 | 11 |
| | G2/M | 22 | 46 | 22 |

*HCT116 Dicer^{ex5} cells were transfected with either miR-210 duplexes, miR-210 containing mismatches at positions 5 and 6 in the seed region (miR-210 mt) or mock transfected as control. 24 hours post-transfection the cells were exposed to hypoxia (1% O₂) or normoxia (21% O₂) for another 24 hours before analyzing cell cycle distribution. The percentage of cells in G1, S or G2/M phase are shown.

As shown in Table 7, the effect of miR-210 was dose dependent and was observable at concentrations as low as 0.5 nM. This data suggests that miR-210 reversed the hypoxia response by overriding cell cycle arrest.

TABLE 7
Cell cycle progression after transfection
with different concentrations of miR-210.*

| | | | | | | | miR-210 |
|---|---|---|---|---|---|---|---|
| | Cell-cycle | | miR-210 | miR-210 | miR-210 | miR-120 | mt |
| | phase | Control | 0.1 nM | 0.5 nM | 1.0 nM | 10 nM | 10 nM |
| Hypoxia | G1 | 70 | 66 | 57 | 52 | 35 | 65 |
| | S | 8 | 12 | 15 | 16 | 19 | 7 |
| | G2/M | 18 | 19 | 24 | 28 | 40 | 24 |

*HCT116 Dicer^{ex5} cells were transfected with increasing concentrations of miR-210 duplex (0.1, 0.5, 1.0, and 10.0 nM) or miR-210 seed region mutant (10.0 nM). 24 hours post-transfection the cells were exposed to hypoxia (1% O₂) for another 48 hours before cell cycle analysis. The percentage of cells in G1, S or G2/M phase are shown.

It was also observed that miR-210 accelerates the G1-S transition under normoxia conditions. As shown in Table 6, the G1 peak is reduced from 45% to 19% and the G2/M peak increased from 28% to 54% compared to mock transfected control cells. Because HIFs are de-stabilized and non-functional under normoxic conditions, this data suggests that miR-210 may act independently of HIFs to regulate cell cycle progression.

The role of endogenous miR-210 in cell cycle progression was investigated by performing loss-of-function analysis using Locked Nucleic Acid (LNA)-modified oligonucleotides that target miR-210 (anti-miR-210) to specifically inhibit miR-210 function. 786-O-pBABE cells were used because this cell line is defective in pVHL function, which stabilizes HIF-α resulting in a constitutively elevated level of miR-210 regardless of the oxygen level. Previous results showed that the G0/G1 accumulation phenotype was easier to measure when the microtubule depolymerizing drug nocodazole was added after transfection to block cells from reentering the cell cycle after mitosis (Linsley, P. S., et al., “Transcripts Targeted by the microRNA-16 Family Cooperatively Regulate Cell Cycle Progression,” Mol. Cell. Biol. 27:2240-2252, 2007). Therefore, cells were treated with nocodazole 30 hours after transfection and the cell-cycle distribution was analyzed 16 hours after nocodazole treatment. Nearly all (greater than 90%) of the cells mock transfected or transfected with a control anti-miR-185 LNA oligonucleotide (that is not known to cause a cell cycle phenotype) accumulated in G2/M phase (4N DNA content) after nocodazole treatment. On the other hand, approximately 23% of anti-miR-210 transfected cells remained in G0/G1 (data not shown), indicating that miR-210 functions as a positive regulator of the G1-S transition.

In summary, this example demonstrates the unexpected result that miR-210 overexpression in tumor cells was able to override hypoxia-induced cell-cycle arrest. Further, because miR-210 overexpression also accelerates the cell cycle under normoxia conditions, the data in this example suggest that miR-210 may act independently of HIFs to regulate the cell cycle. This example also suggests that inhibiting miR-210 expression prevents cells from progressing through the cell cycle under hypoxic conditions, and that inhibitors of miR-210 may have therapeutic benefits.

### Example 5

This example shows that miR-210 reverses the gene expression pattern induced by hypoxia.

Methods

HCT116 Dicer^{ex5} cells were transfected with miR-210 duplexes or HIF-1α siRNA for 24 hours as described in Example 2, then exposed to hypoxia for 24 hours. ME180 cells were transfected using DharmaFect (Dharmacon, Lafayette, Colo.). Microarray analysis was performed as described in Example 1.

Results

To understand the mechanism of miR-210 function under both hypoxic and normoxic conditions, microarray analysis was used to examine gene expression in cells transfected with miR-210 duplexes (gain-of-function experiments). Table 8 shows that transcripts up-regulated by miR-210 under hypoxic conditions overlapped significantly with transcripts down-regulated by hypoxia in HCT116 Dicer^{ex5} cells (p-value=6.3E-12). In contrast, there was no significant overlap between miR-210 up-regulated transcripts and those up-regulated by hypoxia. Similarly, transcripts down-regulated by miR-210 overlapped significantly with transcripts up-regulated by hypoxia (p-value=8.1E-13), whereas there was no significant overlap between miR-210 down-regulated transcripts and transcripts down-regulated by hypoxia.

TABLE 8
Overlap of miR-210 and hypoxia gene expression profiles.*

| Signature Gene | Genes up-regulated | Genes down-regulated |
|---|---|---|
| Sets Compared | by miR-210 | by miR-210 |
| Genes up- | P-value: 1.0 | P-value: 8.1e−13 |
| regulated | miR-210: 2414 genes | miR-210: 1178 genes |
| by hypoxia | hypoxia: 3673 genes | hypoxia: 3281 genes |
| | overlap: 151 genes | overlap: 543 genes |
| Genes down- | P-value: 6.3e−12 | P-value: 1.0 |
| regulated | miR-210: 936 genes | miR-210: 1345 gene |
| by hypoxia | hypoxia: 4149 genes | hypoxia: 5402 genes |
| | overlap: 1629 genes | overlap: 376 genes |

*HCT116 Dicer^{ex5} cells were exposed to hypoxia for 24 hours or transfected with miR-210 duplexes 24 hours prior to hypoxia treatment for an additional 24 hours. RNA was isolated and microarray analysis performed to identify signature genes (P < 0.01).

Further, gene expression patterns observed when miR-210 is overexpressed are similar to those observed when HIF-1α is inhibited by siRNA. For example, as shown in Table 9, the signature gene set up-regulated by miR-210 under hypoxia significantly overlaps the gene set up-regulated by HIF-1α siRNA under hypoxia, and both the up-regulated miR-210 and HIF-1α siRNA gene sets significantly overlap the gene set down-regulated by hypoxia. Likewise, the signature gene set down-regulated by miR-210 under hypoxia significantly overlaps the gene set down-regulated by HIF-1α siRNA under hypoxia, and both the down-regulated miR-210 and HIF-1α siRNA gene sets significantly overlap the gene set up-regulated by hypoxia (Table 9). Taken together, these results indicate that miR-210 negatively regulates a subset of the hypoxia gene expression response, and that the set of genes regulated by miR-210 significantly overlaps the set of genes regulated by siRNA that targets HIF-1α.

TABLE 9
Overlap of gene expression profiles of cells treated
with hypoxia, miR-210 and HIF-1α siRNA.*

| Signature gene | Hypoxia | Hypoxia |
|---|---|---|
| sets compared | up-regulated | down-regulated |
| miR-210 | P-value = 1.0 | P-value = 8.14e−13 |
| up-regulated | | |
| miR-210 | P-value = 6.25e−12 | P-value = 1.0 |
| down-regulated | | |
| HIF-1a siRNA | P-value = 1.0 | P-value = 4.84e−11 |
| up-regulated | | |
| HIF-1a siRNA | P-value = 4.33e−11 | P-value = 0.66 |
| down-regulated | | |

*HCT116 Dicer^{ex5} cells were exposed to hypoxia for 24 hours or they were transfected with miR-210 duplexes or HIF-1α siRNA 24 hours prior to hypoxia treatment for an additional 24 hours. RNA was isolated and microarray analysis performed to identify signature genes (P < 0.01). The p values were calculated using the Log10 Wilcoxon signed-rank test, and show the probability that the indicated gene sets overlap by chance.

To determine if inhibiting endogenous miR-210 function would affect gene expression profiles, ME-180 cells were transfected with miR-210 or anti-miR-210 duplexes as described in Example 2. The cells were transfected with siRNA targeting luciferase as a control. Twenty-four hours after transfection, the cells were exposed to hypoxia for another 24 hours before harvesting RNA for microarray analysis. Consensus genes that were down-regulated by miR-210 under hypoxic conditions (see Table 10) were up-regulated by treatment of cells with anti-miR-210. The up-regulation of target genes by anti-miR-210 was statistically significant when compared to control cells treated with siRNA to luciferase (p-value=1.44E-04).

In summary, the data presented in this example shows that miR-210 negatively regulates genes that are up-regulated by hypoxia and that inhibiting miR-210 reverses the negative regulation of genes by miR-210 under hypoxic conditions. Further, miR-210 may act independently of HIF-1α, because the set of genes down-regulated by miR-210 overlaps the set of genes down-regulated when HIF-1α is inhibited by siRNA under hypoxic conditions.

### Example 6

This example shows that silencing Mnt with siRNA phenocopies miR-210 overexpression.

Methods

miR-210 consensus down-regulated transcripts. HCT116 Dicer^{ex5} cells, RKO Dicer^{ex5}, and DLD-1 Dicerex5 cells were transfected with miR-210 duplexes, and gene expression signatures were determined at 24 hours. The intersection signature (p<0.01) between any two of the cell lines was identified. Transcripts in the intersection signature that were also regulated (p<0.05) at 6 hours in HCT116 Dicer^{ex5} cells were defined as miR-210 consensus down-regulated transcripts.

SiRNAs were transfected into HCT116 Dicer^{ex5} cells as described in Example 2. The siRNAs targeting Mnt comprise the guide strands of SEQ ID NOs:15-17. Twenty-four hours after transfection, the cells were exposed to hypoxia or normoxia for 48 hours before harvesting RNA for cell cycle analysis. Cell cycle analysis was performed as described in Example 4.

Immunoblotting was performed as described (Jackson, A. L., et al., “Widespread siRNA ‘Off-Target’ Transcript Silencing Mediated by Seed Region Sequence

Complementarity,” RNA 12:1179-1187, 2006). Anti-Mnt monoclonal antibody (AB53487) was purchased from Abcam (Cambridge, Mass.). HCT116 Dicer^{ex5} cells were transfected with Luciferase control siRNA, miR-210 or Mnt siRNA. Twenty-four hours after transfection, the cells were exposed to hypoxia or normoxia for another 24 hours before analysis of Mnt protein expression by western blot analysis. Protein expression was normalized to Actin for each treatment. Anti-Mnt monoclonal antibody (AB53487) was purchased from Abcam (Cambridge, Mass.).

Results

As shown in Table 10, the gene expression analysis described in Example 5 identified 31 transcripts that were downregulated by miR-210 overexpression 6 hours after transfection. These transcripts also contain miR-210 complementary hexamers in their 3′-UTR regions and are therefore likely to represent direct targets of miR-210.

TABLE 10
miR-210 consensus down-regulated transcripts^{a}

| Entrez | | | | |
|---|---|---|---|---|
| GeneID | | | Mean | Mean p-value |
| (Locus | Accession | Gene | expression fold | for expression |
| Link) | Number | Symbol | change at 24 hr | change at 24 hr |
| 103 | NM_015840 | ADAR | −1.457 | 4.74E−04 |
| 9334 | NM_004776 | B4GALT5 | −1.446 | 8.36E−04 |
| 1944 | NM_004952 | EFNA3 | −1.874 | 3.50E−06 |
| 79071 | NM_024090 | ELOVL6 | −2.610 | 2.20E−16 |
| 10447 | NM_014888 | FAM3C | −1.973 | 4.44E−10 |
| 79443 | NM_024513 | FYCO1 | −1.884 | 2.79E−08 |
| 26035 | AB020643 | GLCE | −3.265 | 2.24E−09 |
| 9759 | NM_006037 | HDAC4 | −2.267 | 1.44E−15 |
| 3638 | NM_005542 | INSIG1 | −1.518 | 8.10E−05 |
| 23479 | NM_014301 | ISCU | −2.631 | 2.05E−14 |
| 3726 | NM_002229 | JUNB | −1.136 | 3.08E−01 |
| 51603 | NM_015935 | KIAA0859 | −1.931 | 1.07E−08 |
| 3927 | NM_006148 | LASP1 | −2.059 | 2.24E−11 |
| 10186 | NM_005780 | LHFP | −2.179 | 2.92E−08 |
| 9477 | NM_004275 | MED20 | −1.922 | 2.00E−06 |
| 23295 | AB011116 | MGRN1 | −3.347 | 1.29E−22 |
| 58526 | NM_021242 | MID1IP1 | −1.712 | 1.00E−05 |
| 4335 | NM_020310 | MNT | −1.363 | 1.38E−02 |
| 7994 | NM_006766 | MYST3 | −1.692 | 4.45E−05 |
| 11051 | NM_007006 | NUDT21 | −2.215 | 5.47E−21 |
| 54776 | NM_017607 | PPP1R12C | −1.675 | 6.19E−04 |
| 11099 | NM_007039 | PTPN21 | −1.378 | 1.54E−02 |
| 6388 | NM_006923 | SDF2 | −2.280 | 3.32E−11 |
| 83959 | NM_032034 | SLC4A11 | −1.492 | 9.58E−03 |
| 200734 | NM_181784 | SPRED2 | −1.856 | 9.00E−06 |
| 6845 | NM_005638 | SYBL1 | −3.749 | 7.47E−28 |
| 54386 | NM_018975 | TERF2IP | −2.516 | 8.75E−13 |
| 23534 | NM_012470 | TNPO3 | −2.425 | 2.80E−10 |
| 84969 | NM_032883 | TOX2 | −1.735 | 5.10E−05 |
| 58485 | NM_021210 | TRAPPC1 | −2.447 | 8.29E−16 |
| 84878 | NM_032792 | ZBTB45 | −1.789 | 1.69E−04 |

^{a}A consensus set of genes that were significantly down-regulated by miR-210 in multiple cell lines. HCT116 Dicer^{ex5} cells, RKO Dicer^{ex5} cells and DLD-1 Dicer^{ex5} cells were transfected with miR-210 duplexes, and gene expression signatures were determined at 24 hrs. The intersection signature (p < 0.01) between any two of the cell lines was identified. Transcripts in the intersection signature that were also regulated (p < 0.05) at 6 hrs in HCT116 Dicerex5 cells were defined as miR-210 consensus down-regulated transcripts. The 3′UTR of these transcripts also contained sequences matching the miR210 seed region. The mean expression fold change of each gene at 24 hr and the corresponding p-value in response to miR-210 duplex transfection in the 3 cell lines are listed in the last two columns.

Specific pools of siRNAs (three siRNAs targeting the same gene) for each of the targets in Table 10 were transfected into HCT116 Dicer^{ex5} cells and analyzed for their effects on cell cycle progression under hypoxic conditions. Mnt, a basic helix-loop-helix transcription factor (Hurlin, P. J., et al., “Mnt, A Novel Max-Interacting Protein Is Coexpressed With Myc in Proliferating Cells and Mediates Repression at Myc Binding Sites,” Genes Dev. 11:44-58, 1997; Hurlin, P. J., et al., “Deletion of Mnt Leads to Disrupted Cell Cycle Control and Tumorigenesis,” Embo. J. 22:4584-4596, 2003), was the most prominent target whose silencing phenocopied miR-210 gain-of-function. As shown in Table 11, cells treated with miR-210 or Mnt siRNA showed a decrease in cells in G1 and an increase in cells in S phase compared to cells transfected with a control Luc siRNA.

TABLE 11
Cell Cycle Progression After Transfection
With miR-210 and Mnt siRNA.*

| | Cell-cycle phase | Luc siRNA | miR-210 | Mnt siRNA |
|---|---|---|---|---|
| Normoxia | G1 (%) | 68 | 40 | 44 |
| | S (%) | 11 | 14 | 21 |
| | G2/M (%) | 17 | 36 | 22 |
| Hypoxia | G1 (%) | 80 | 46 | 53 |
| | S (%) | 5 | 13 | 16 |
| | G2/M (%) | 11 | 29 | 16 |

*HCT116 Dicer^{ex5} cells were transfected with Luciferase control siRNA, miR-210 duplexes, or Mnt siRNA. 24 hours post-transfection the cells were exposed to hypoxia (1% O₂) or normoxia (21% O₂) for another 48 hours before analyzing cell cycle distribution. The percentage of cells in G1, S, or G2/M phase is shown.

The cell cycle effect under hypoxia was observed with multiple siRNAs against Mnt (SEQ ID NOs:15-17). The use of multiple siRNAs that target the same mRNA tends to exclude off-target effects of individual siRNA molecules (Jackson, A. L., et al., “Expression Profiling Reveals Off-Target Gene Regulation by RNAi,” Nat. Biotechnol. 21:635-637, 2003).

Consistent with miR-210 directly targeting Mnt, the 3′UTR of Mnt mRNA contains four potential consensus sites matching the miR-210 seed region. Further, miR-210 overexpression reduced Mnt protein levels under both normoxic (by 33%) and hypoxic (by 41%) conditions (data not shown). Hypoxia elevated the Mnt protein level by 76% in Luciferase siRNA control transfected cells.

The effect Mnt silencing had on gene expression was compared to miR-210 overexpression. As shown in Tables 12 and 13, there was significant positive overlap between the signature genes of miR-210 and Mnt siRNA in HFF-pBABE cells under both normoxic (Table 12) and hypoxic (Table 13) conditions.

TABLE 12
Overlap of miR-210 and Mnt siRNA gene
expression profiles under normoxia.*

| Signature Gene | Genes up-regulated | Genes down-regulated |
|---|---|---|
| Sets Compared | by miR-210 | by miR-210 |
| Genes up- | P-value: 1.7e−11 | P-value: 1.0 |
| regulated | miR-210: 711 genes | miR-210: 1200 genes |
| by Mnt siRNA | Mnt siRNA: 1367 genes | Mnt siRNA: 860 genes |
| | overlap: 536 genes | overlap: 47 genes |
| Genes down- | P-value: 1.0 | P-value: 1.4e−12 |
| regulated | miR-210: 789 genes | miR-210: 575 gene |
| by Mnt siRNA | Mnt siRNA: 1832 genes | Mnt siRNA: 622 genes |
| | overlap: 71 genes | overlap: 285 genes |

*HFF-pBABE cells were transfected with miR-210 or Mnt siRNA for 48 hours under normoxic conditions, and microarray analysis was performed to identify signature genes (P < 0.01).

TABLE 13
Overlap of miR-210 and Mnt siRNA gene
expression profiles under hypoxia.*

| Signature Gene | Genes up-regulated | Genes down-regulated |
|---|---|---|
| Sets Compared | by miR-210 | by miR-210 |
| Genes up- | P-value: 0e00 | P-value: 1.0e00 |
| regulated | miR-210: 621 genes | miR-210: 864 genes |
| by Mnt siRNA | Mnt siRNA: 859 genes | Mnt siRNA: 1083 genes |
| | overlap: 300 genes | overlap: 57 genes |
| Genes down- | P-value: 1.0e00 | P-value: 0e00 |
| regulated | miR-210: 1279 genes | miR-210: 951 gene |
| by Mnt siRNA | Mnt siRNA: 1091 genes | Mnt siRNA: 744 genes |
| | overlap: 68 genes | overlap: 396 genes |

*HFF-pBABE cells were transfected with miR-210 or Mnt siRNA for 48 hours under hypoxic conditions, and microarray analysis was performed to identify signature genes (P < 0.01).

In summary, the data presented in this example shows that Mnt is down-regulated by miR-210. This example also shows that inhibiting Mnt expression with siRNA produces similar effects on the cell cycle as overexpression of miR-210. Further, the data in this example shows that the set of genes regulated by miR-210 and Mnt siRNA show significant overlap under normoxia and hypoxia.

### Example 7

This example shows that miR-210 regulates the cell cycle through c-Myc.

Methods

HCT116 Dicer^{ex5} cells and human foreskin fibroblasts (HFFs) were transfected with miRNA and siRNA duplexes as described in Example 2. Cell cycle analysis was performed as described in Example 4. For co-transfection experiments, 50 nM (final concentration) of Luciferase control siRNA or Myc siRNA was combined with 10 nM (final concentration) of Luciferase siRNA, miR-210 (SEQ ID NO:1) or miR-210 seed region mutant (miR-210 mt; SEQ ID NO:5). The siRNAs targeting Myc comprise the guide strands of SEQ ID NOs:18-20. Microarray analysis was performed as described in Example 1. HFF-pBABE (empty vector) and HFF-c-Myc cells were described previously (Benanti, J. A., et al., “Epigenetic Down-Regulation of ARF Expression Is a Selection Step in Immortalization of Human Fibroblasts by c-Myc,” Mol. Cancer. Res. 5:1181-1189, 2007). The level of c-Myc protein expressed by the HFF-c-Myc cells was measured by Western Blot as described in Benanti et al. (“Epigenetic Down-Regulation of ARF Expression Is a Selection Step in Immortalization of Human Fibroblasts by c-Myc,” Mol. Cancer. Res. 5:1181-1189, 2007).

For the synthetic lethal experiments, HFF-pBABE or HFF-c-Myc cells were mock transfected or transfected with Luciferase control siRNA, miR-210 duplexes, Mnt siRNA, or KIF11 siRNA, as described in Example 2. Images were captured 4 days post-transfection, and the number of live and dead cells, as determined by visual inspection of the images, was counted. Data are presented as the average value from triplicate experiments with standard deviation error bars.

Results

The previous example showed that miR-210 inhibits Mnt expression. Mnt is a Max-interacting transcriptional repressor that functions as a c-Myc antagonist (Hurlin, P. J., et al., “Deletion of Mnt Leads to Disrupted Cell Cycle Control and Tumorigenesis,” Embo. J. 22:4584-4596, 2003; Walker, W., et al., “Mnt-Max to Myc-Max Complex Switching Regulates Cell Cycle Entry,” J. Cell Biol. 169:405-413, 2005). Therefore, miR-210 could regulate cell cycle progression by indirect activation of c-Myc. As shown in Table 14, knockdown of c-Myc with siRNA in HCT116 Dicer^{ex5} cells impaired the ability of miR-210 to override hypoxia induced G1-S arrest, while a Luc control siRNA had no obvious effect on miR-210 phenotype. The maximal silencing efficiency of c-Myc siRNA was only about 50% (data not shown), and the incomplete knockdown of c-Myc could explain the residual effect of miR-210 on hypoxia induced G1-S arrest in the c-Myc silenced cells. Similarly, under normoxia conditions, c-Myc siRNA but not control Luc siRNA compromised miR-210's ability to drive cells into S phase (Table 14). Collectively, these data illustrate that the effect on cell cycle induced by miR-210 is largely dependent on c-Myc.

TABLE 14
The effect of miR-210 and c-Myc on the cell cycle.*

| | Percentage of cells in S-phase | | | | | |
|---|---|---|---|---|---|---|
| Co- | Transfected siRNA | | | | | |
| transfected | Luc siRNA + | | | c-Myc siRNA + | | |
| duplex | | miR- | miR-210 | Luc | miR- | miR-210 |
| RNA | None | 210 | mt | siRNA | 210 | mt |
| Normoxia | 14.6% | 27.2% | 14.4% | 8.78% | 12.3% | 7.22% |
| Hypoxia | 5.73% | 23.2% | 6.29% | 4.86% | 9.93% | 5.16% |

*miR-210 and c-Myc siRNA duplexes were transfected into HCT116 Dicer^{ex5} cells. Luc siRNA and miR-210 seed region mutant (miR-210 mt) were used as controls. BrdU incorporation was determined using flow cytometry 48 hours after hypoxia. Percentage of cells in S-phase is shown.

Primary human foreskin fibroblasts (HFFs) were used to determine the effect of miR-210 gain-of-function, c-Myc-overexpression, and Mnt loss-of-function on gene expression profiles. HFFs allow c-Myc overexpression without triggering a senescent response (Benanti, J. A., et al., “Epigenetic Down-Regulation of ARF Expression Is a Selection Step in Immortalization of Human Fibroblasts by c-Myc,” Mol. Cancer. Res. 5:1181-1189, 2007). In response to c-Myc, HFFs exhibit many of the growth phenotypes that characterize c-Myc function, such as increased rRNA and DNA synthesis (Dominguez-Sola, D., et al., “Non-Transcriptional Control of DNA Replication by c-Myc,” Nature 448:445-451, 2007; Grandori, C., et al., “c-Myc Binds to Human Ribosomal DNA and Stimulates Transcription of rRNA Genes by RNA Polymerase I,” Nat. Cell Biol. 7:311-318, 2005). A Myc overexpression signature was first generated by comparing three independent sets of HFFs with and without c-Myc constitutively expressed from a retroviral vector, pBabe. A total of 1063 genes were up regulated and 981 down-regulated in all three matched pair of cells (data not shown). As shown in Table 15, miR-210 induced 22% of the genes up-regulated by c-Myc (353 out of 1550 genes). The probability of observing this level of overlap by chance is less than 3.5×10E-11. Similarly, the genes down-regulated by c-Myc overexpression and miR-210 overexpression also overlapped significantly (p-value=4.0×10E-12).

TABLE 15
Overlap of miR-210 and c-Myc gene expression
profiles under normoxia.*

| Signature Gene | Genes up-regulated | Genes down-regulated |
|---|---|---|
| Sets Compared | by miR-210 | by miR-210 |
| Genes up- | P-value: 3.5e−11 | P-value: 1.0e00 |
| regulated | miR-210: 710 genes | miR-210: 926 genes |
| by c-Myc | c-Myc: 1550 genes | c-Myc: 870 genes |
| | overlap: 353 genes | overlap: 37 genes |
| Genes down- | P-value: 1.0e00 | P-value: 4.0e−12 |
| regulated | miR-210: 867 genes | miR-210: 704 genes |
| by c-Myc | c-Myc: 1789 genes | c-Myc: 630 genes |
| | overlap: 114 genes | overlap: 277 genes |

*HFF-pBABE cells were transfected with miR-210 duplexes or pBABE-c-Myc retroviral vector for 48 hours under normoxic conditions, and microarray analysis was performed to identify signature genes (P < 0.01). c-Myc signature genes were determined by comparing the gene expression profiles of HFF-pBABE and HFF-pBABE-c-Myc cells.

2D clustering was used to compare the gene expression profiles of miR-210 overexpression, c-Myc overexpression, and Mnt knockdown in HFF cells. Myc siRNA was used as a positive control for the Myc signature and Luc siRNA was used as a negative control. Two hundred eighty-four (284) genes were either significantly up-regulated or down-regulated (P<0.01). The 284 gene set contained three clusters. Cluster 1 contained genes down-regulated by Myc overexpression but up-regulated by miR-210 overexpression and Mnt siRNA knockdown. Cluster 2 includes genes that were up-regulated by c-Myc overexpression, miR-210 overexpression, and Mnt siRNA knockdown. Cluster 3 includes genes that were down-regulated by Myc overexpression, miR-210 overexpression, and Mnt siRNA knockdown.

Functional annotation of the genes in each cluster revealed that cluster 1 was highly enriched in genes potentially involved in metastasis and angiogenesis. The majority of genes in cluster 2 (upregulated by Myc, miR-210 and Mnt siRNA) functioned in Pol I, II, and III transcription, or rRNA processing and metabolism, consistent with the Myc effect on cell growth. A smaller fraction of the genes are involved in DNA damage, mitochondrial function and apoptosis, consistent with the known effects of Myc on DNA replication and sensitization to apoptotic stimuli. Many genes in cluster 3 (downregulated by Myc, miR-210 and Mnt siRNA) are involved in cytoskeletal dynamics and extracellular matrix, including Thrombospondin, which is known to be inhibited by Myc and whose down-regulation promotes angiogenesis.

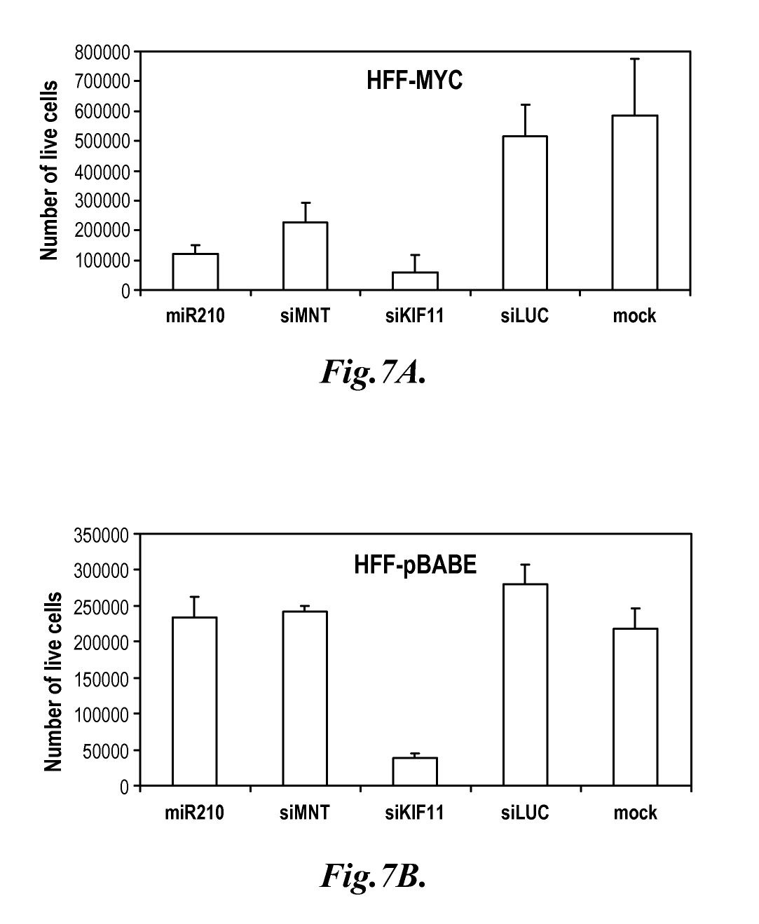

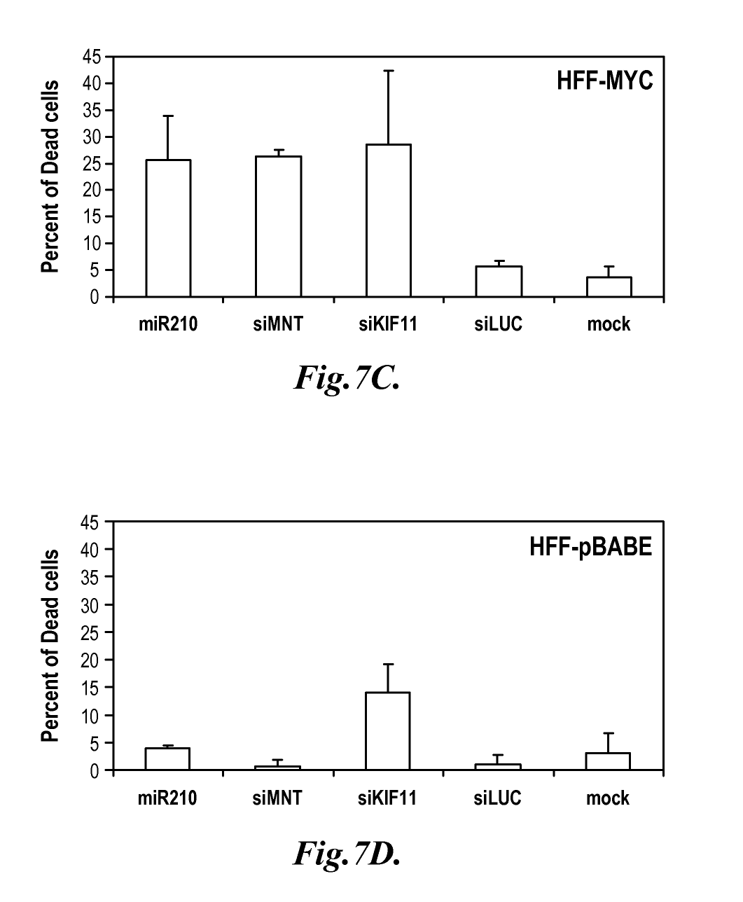

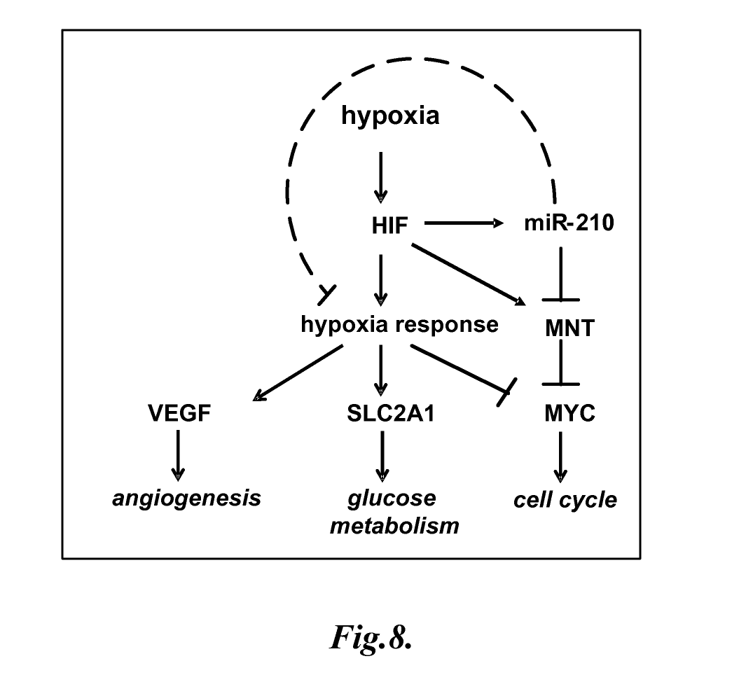

Excessive levels of c-Myc have been shown to enhance cell death under certain conditions (Evan, G. I., et al., “Induction of Apoptosis in Fibroblasts by c-Myc Protein,” Cell 69:119-128, 1992; Nilsson, J. A., and J. L. Cleveland, “Myc Pathways Provoking Cell Suicide and Cancer,” Oncogene 22:9007-9021, 2003). Therefore, HFF-Myc cells were transfected with microRNAs to determine if any microRNAs enhanced cell death in cells overexpressing c-Myc. miR-210 was identified as a microRNA that increased cell death in HFF-Myc cells. As shown in FIG. 7, transfection of HFF-Myc cells with miR-210 under normoxia conditions decreased the number of live cells (FIG. 7A) and increased the percentage of dead cells (FIG. 7C) compared to mock transfected controls. The increase in cell death was not observed in HFF-pBABE (empty vector control) cells (FIGS. 7B, 7D). Silencing of Mnt with siRNA also decreased the number of live cells and increased the percentage of dead cells compared to Luc siRNA transfected control cells (FIGS. 7A, 7C). KIF11 siRNA was used as a positive control for transfection efficiency as a reduction in KIF11 levels causes mitotic arrest (Weil et al., Biotechniques 33:1244-1248, 2002). Thus, both overexpression of miR-210 and inhibition of Mnt increased cell death in cells that overexpress c-Myc. While not wishing to be bound by theory, FIG. 8 shows a model of how the hypoxia, miR-210, and c-Myc genetic pathways may interact.

In summary, the data presented in this example show that the genes regulated by miR-210, c-Myc, and Mnt overlap. An important and unexpected finding of the present invention is that miR-210 induces cell death in cells that overexpress c-Myc. Further, inhibition of Mnt also increased cell death in cells that overexpressed c-Myc, indicating that miR-210 may be a negative regulator of Mnt. These findings are useful for treatment of tumor cells that overexpress c-Myc.

While the preferred embodiment of the invention has been illustrated and described, it will be appreciated that various changes can be made therein without departing from the spirit and scope of the invention.

1. A method for determining a hypoxic state in tumor cells obtained from a subject, comprising:
(a) measuring the level of miR-210 in tumor cells obtained from a tumor in a subject; and
(b) comparing the level of miR-210 with a hypoxia reference value, wherein a level greater than the hypoxia reference value is indicative of a hypoxic state in the tumor cells.

2. The method of claim 1, wherein the hypoxia reference value is selected from the group consisting of:
(a) the level of miR-210 in non-tumor cells obtained from the subject;
(b) the level of miR-210 in cells obtained from a plurality of non-hypoxic tumor samples from one or more subjects, and
(c) the level of miR-210 in non-tumor cells from one or more subjects.

3. The method of claim 1, wherein the tumor cells are obtained from a tumor type selected from the group consisting of breast, kidney, lung, and melanoma cancers.

4. The method of claim 1, wherein a hypoxic state is predictive of metastasis of the cancer.

5. A method for predicting the likelihood of metastasis of a tumor in a subject, comprising:
(a) measuring the level of miR-210 in tumor cells obtained from a tumor in a subject; and
(b) comparing the measured level of miR-210 with a metastasis reference value, wherein a level of miR-210 equal to or greater than the metastasis reference value is predictive of metastasis of the tumor in the subject.

6. The method of claim 5, wherein the metastasis reference value is the level of miR-210 measured in cells from non-tumor tissue in one or more subjects.

7. The method of claim 5, wherein the metastasis reference value is the median expression level of miR-210 measured in cells from a plurality of primary tumors from one or more subjects with no metastasis for at least five years.

8. The method of claim 5, wherein the tumor is selected from the group consisting of breast, kidney, lung, and melanoma cancers.

9. A method of inhibiting tumor cell proliferation, comprising:
(a) measuring the level of Myc protein or nucleic acid in a tumor cell sample;
(b) comparing the measured level of Myc protein or nucleic acid with a corresponding Myc reference value; and
(c) contacting the tumor cells having a level of Myc equal to or greater than the Myc reference value with an amount of a short interfering nucleic acid (siNA) comprising an miR-210 sequence and is effective to inhibit the proliferation of tumor cells, wherein said siNA comprises a guide strand nucleotide sequence wherein at least 6 contiguous nucleotides are identical to 6 contiguous nucleotides of SEQ ID NO:4.

10. The method of claim 9, wherein step (a) comprises measuring at least one of:
(i) a polynucleotide having at least 95% sequence identity to the polynucleotide of SEQ ID NO:23, SEQ ID NO:25, or SEQ ID NO:27, or a variant or polymorphism thereof; or
(ii) a polypeptide having at least 95% sequence identity to the polypeptide of SEQ ID NO:24, SEQ ID NO:26, or SEQ ID NO:28, or an isoform thereof.

11. The method of claim 9, wherein the siNA guide strand comprises a contiguous nucleotide sequence of at least 18 nucleotides, wherein said guide strand comprises a seed region consisting of nucleotide positions 1 to 12, and wherein position 1 represents the 5′-end of said guide strand.

12. The method of claim 9, wherein the Myc reference value is selected from the group consisting of:
(i) the level of Myc in non-tumor cells obtained from one or more subjects;
(ii) the level of Myc in tumor cells obtained from one or more subjects;
(iii) the level of Myc in cells obtained from a plurality of tumors in one or more subjects;
(iv) the level of Myc in one or more tumor cell lines; and
(iv) the level of Myc in non-tumor cells transduced with a Myc expression vector.

13. The method of claim 9, wherein the tumor is selected from the group of cancers consisting of lymphoma, neuroblastoma, medulloblastoma, glioblastomas, rhabdomyosarcomas, hepatocellular carcinoma, lung cancer, breast cancer, colon cancer, prostate cancer, pancreatic cancer, skin cancer, and ovarian cancer.

14. A method of reducing the tumor burden in a subject, comprising contacting a plurality of tumor cells with an amount of a small interfering nucleic acid (siNA) effective to reduce tumor burden in the subject, wherein said siNA comprises a guide strand nucleotide sequence of at least contiguous 18 nucleotides, wherein said guide strand comprises a seed region consisting of nucleotide positions 1 to 12, wherein position 1 represents the 5′-end of said guide strand, and wherein said seed region comprises a nucleotide sequence of at least 6 contiguous nucleotides that is identical to 6 contiguous nucleotides of SEQ ID NO:4.

15. The method of claim 14, wherein the tumor cells express c-Myc, N-Myc, or L-Myc.

16. The method of claim 14, wherein the tumor is selected from the group of cancers consisting of lymphoma, neuroblastoma, medulloblastoma, glioblastomas, rhabdomyosarcomas, hepatocellular carcinoma, lung cancer, breast cancer, colon cancer, prostate cancer, pancreatic cancer, skin cancer, and ovarian cancer.

17. A method of inhibiting the proliferation of tumor cells comprising:
(a) measuring the level of Myc protein or nucleic acid in the tumor cells;
(b) comparing the measured level of Myc protein or nucleic acid in the tumor cells with a corresponding Myc reference value; and
(c) contacting the tumor cells having a level of Myc equal to or greater than the Myc reference value with an amount of an inhibitor of the expression or activity of:
(i) a polypeptide having at least 95% identity to the polypeptide set forth in SEQ ID NO:30; or
(ii) a polynucleotide having at least 95% identity to the polynucleotide set forth in SEQ ID NO:29;
that is effective to inhibit proliferation of the tumor cells.

18. A method of inhibiting tumor cell proliferation in a subject, comprising:
(a) measuring the level of miR-210 in tumor cells obtained from the subject;
(b) comparing the measured level of miR-210 with a hypoxia reference value; wherein measured levels equal to or greater than the hypoxia reference value indicate the tumor cells are hypoxic; and
(c) contacting the tumor cells in the subject with an inhibitor of the hypoxia response pathway; thereby inhibiting the proliferation of tumor cells in the subject.

19. The method of claim 18, wherein the hypoxia response pathway comprises a polypeptide selected from the group consisting of HIF-1α, HIF-1β, and HIF-2α.

20. The method of claim 18, wherein the inhibitor of the hypoxia response pathway inhibits the expression or activity of the polynucleotide set forth in SEQ ID NO:29 or the polypeptide set forth in SEQ ID NO:30.

21. The method of claim 18, wherein the hypoxia reference value is selected from the group consisting of:
(i) the level of miR-210 in non-tumor cells obtained from the subject;
(ii) the level of miR-210 in cells obtained from a plurality of non-hypoxic tumor samples from one or more subjects,
(iii) the level of miR-210 in non-tumor cells obtained from one or more subjects; and
(iv) the level of miR-210 in cells obtained from one or more non-hypoxic tumor cell lines.

22. A method of inhibiting tumor cell proliferation in a subject, comprising:
(a) measuring the level of miR-210 in tumor cells obtained from a subject;
(b) comparing the measured level of miR-210 with a hypoxia reference value; wherein measured levels equal to or greater than the hypoxia reference value indicate the tumor cells are hypoxic; and
(c) contacting the tumor cells in the subject with a miR-210 inhibitor, thereby inhibiting the proliferation of tumor cells in the subject.

23. The method of claim 22, wherein the miR-210 inhibitor comprises an oligonucleotide complementary to at least 6 contiguous nucleotides of SEQ ID NO:4.
